(19) United States
(12) Patent Application Publication
(10) Pub. No.: US 2016/0122821 A1
(43) Pub. Date: May 5, 2016

(54) GENETIC MARKERS OF ANTIPSYCHOTIC RESPONSE

(71) Applicant: SUREGENE, LLC, Louisville, KY (US)

(72) Inventors: Qian LIU, Louisville, KY (US); Mark D. BRENNAN, Jeffersonville, IN (US)

(73) Assignee: SUREGENE, LLC, Louisville, KY (US)

(21) Appl. No.: 14/896,443

(22) PCT Filed: Jun. 10, 2014
(86) PCT No.: PCT/US14/41617

§ 371 Date: Dec. 7, 2015

Related U.S. Application Data
(60) Provisional application No. 61/833,257, filed on Jun. 10, 2013.

(51) Int. Cl. C12Q 1/68; A61K 31/496; A61K 31/5415; A61K 31/554; A61K 31/519; A61K 31/551

(52) U.S. Cl. CPC C12Q 1/6883; A61K 31/519; A61K 31/551; A61K 31/5415; A61K 31/554; A61K 31/496; C12Q 2600/106; C12Q 2600/156; C12Q 2600/172

(57) ABSTRACT

Provided herein are genetic markers for predicting response to antipsychotic treatment. Identification of the disclosed SNPs can be used to predict response to antipsychotic drugs in patients suffering from schizophrenia.

This application claims the benefit of U.S. Provisional Patent Application No. 61/833,257, filed Jun. 10, 2013, the entirety of which is incorporated herein by reference.

This invention was made with government support under Grant No. MH078437 awarded by the National Institutes of Health. The government has certain rights in the invention.

### BACKGROUND OF THE INVENTION

1. Field of the Invention

The present invention relates generally to the fields of medicine, genetics, and psychiatry. More particularly, it concerns genetic markers that are associated with response to antipsychotic treatments.

2. Description of Related Art

The schizophrenia spectrum disorders (SSDs) include schizophrenia (SZ), schizotypal personality disorder (SPD), and/or schizoaffective disorder (SD). Schizophrenia (SZ) is considered a clinical syndrome, and is probably a constellation of several pathologies. Substantial heterogeneity is seen between cases, which is thought to reflect multiple overlapping etiologic factors, including both genetic and environmental contributions. SD is characterized by the presence of affective (depressive or manic) symptoms and schizophrenic symptoms within the same, uninterrupted episode of illness. SPD is characterized by a pervasive pattern of social and interpersonal deficits marked by acute discomfort with, and reduced capacity for, close relationships as well as by cognitive or perceptual distortions and eccentricities of behavior, beginning by early adulthood and present in a variety of contexts.

Various genes and chromosomes have been implicated in etiology of SZ. Many studies have suggested the presence of one or more important genes relating to SZ on most or all of the autosomes (Williams et al., 1999; Fallin et al., 2005; Badner et al., 2002; Cooper-Casey et al., 2005; Devlin et al., 2002; Fallin et al., 2003; Jablensky, 2006; Kirov et al., 2005; Norton et al., 2006; Owen et al., 2004). However, none of these prior studies have used high resolution genetic association methods to systematically compare genes involved in response to treatments for SSD, e.g., using anti-psychotics. Neither have any of these studies demonstrated that genetic polymorphisms in the genes defined herein are important in response to anti-psychotics.

Due to the severity of these disorders, especially the negative impact of a psychotic episode on a patient, and the diminishing recovery after each psychotic episode, there is a need to more conclusively identify individuals who will respond best to specific therapies, and/or who is likely to suffer the most severe side effects, to determine appropriate therapies based on genotypic subtype.

### SUMMARY OF THE INVENTION

Results detailed in the instant application identify over 6,000 SNPs in genes impacting disease risk, disease presentation, and, particularly, response to antipsychotics drug treatment. Thus, in some embodiments methods are provided for detecting the presence of a polymorphism in and administering a treatment to a human subject, comprising (a) obtaining a genomic sample from a human subject having or at risk of developing SZ; (b) detecting the haplotype tagged by an allele selected from those provided in the tables herein; (c) identifying the subject having the haplotype tagged by the allele as likely (or unlikely) to have an improved response to a therapeutic as compared to a control subject; and (d) administering an appropriate treatment to the subject based on this identification.

In one embodiment is provided a method of detecting the presence of a polymorphism in and administering a treatment to a human subject, the method comprising (a) obtaining a genomic sample from a human subject having or at risk of developing SZ; (b) detecting the haplotype tagged by an allele selected from those provided in Table 1A in the genomic sample; (c) identifying the subject having the haplotype tagged by the allele provided in Table 1A in the genomic sample as likely to have an improved response to olanzapine as compared to control subject; and (d) administering a treatment comprising olanzapine to the subject with the haplotype tagged by the allele provided in Table 1A. In certain aspects, the method further comprises detecting the haplotype tagged by two or more alleles (e.g., 1, 2, 3, 4, 5, 6, 7, 8, 9 or more alleles) selected from those provided in Table 1A. In one aspect, said two or more alleles are alleles from two or more different correlated clusters selected from those provided in Table 6.

In a further embodiment, there is provided a method of detecting the presence of a polymorphism in and administering a treatment to a human subject, the method comprising (a) obtaining a genomic sample from a human subject having or at risk of developing SZ; (b) detecting the haplotype tagged by an allele selected from those provided in Table 1B in the genomic sample; (c) identifying the subject having the haplotype tagged by the allele provided in Table 1B in the genomic sample as likely to have a poor response to olanzapine as compared to control subject; and (d) administering an antipsychotic treatment other than olanzapine to the subject with the haplotype tagged by the allele provided in Table 1B. In certain aspects, the method further comprises detecting the haplotype tagged by two or more alleles (e.g., 1, 2, 3, 4, 5, 6, 7, 8, 9 or more alleles) selected from those provided in Table 1B. In one aspect, said two or more alleles are alleles from two or more different correlated clusters selected from those provided in Table 6. In certain aspects, the method comprises administering perphenazine, quetiapine, risperidone or ziprasidone to the subject.

Thus, in some aspects a method is provided for detecting the presence of a polymorphism in the CSMD1 or PTPRN2 gene and administering a treatment to a human subject, the method comprising (a) obtaining a genomic sample from a human subject having or at risk of developing SZ; (b) detecting the haplotype tagged by the “A” allele of rs17070785 or the haplotype tagged by the “C” allele of rs221253 in the genomic sample; (c) identifying the subject having the haplotype tagged by the “A” allele of rs17070785 or the haplotype tagged by the “C” allele of rs221253 in the genomic sample as likely to have an improved response to olanzapine as compared to control subject; and (d) administering a treatment comprising olanzapine to the subject with the haplotype tagged by the “A” allele of rs17070785 or the haplotype tagged by the “C” allele of rs221253.

In a further aspect there is provided a method of detecting the presence of a polymorphism in the PLAGL1 gene and administering an antipsychotic treatment to a human subject, the method comprising (a) obtaining a genomic sample from a human subject having or at risk of developing SZ; (b) detecting the haplotype tagged by the “C” allele of rs2247408 or the haplotype tagged by the “A” allele of rs3819811 in the genomic sample; (c) identifying the subject having the haplotype tagged by the “C” allele of rs2247408 or the haplotype tagged by the “A” allele of rs3819811 in the genomic sample as likely to have a poor response to olanzapine as compared to control subject; and (d) administering an antipsychotic treatment other than olanzapine to the subject with the haplotype tagged by the “C” allele of rs2247408 or the haplotype tagged by the “A” allele of rs3819811. In certain aspects, the method comprises administering perphenazine, quetiapine, risperidone or ziprasidone to the subject.

In a further embodiment, the present invention provides a method of detecting the presence of a polymorphism in and administering a treatment to a human subject, the method comprising (a) obtaining a genomic sample from a human subject having or at risk of developing SZ; (b) detecting the haplotype tagged by an allele selected from those provided in Table 2A in the genomic sample; (c) identifying the subject having the haplotype tagged by the allele provided in Table 2A in the genomic sample as likely to have an improved response to perphenazine as compared to control subject; and (d) administering a treatment comprising perphenazine to the subject with the haplotype tagged by the allele provided in Table 2A. In certain aspects, the method further comprises detecting the haplotype tagged by two or more alleles (e.g., 1, 2, 3, 4, 5, 6, 7, 8, 9 or more alleles) selected from those provided in Table 2A. In one aspect, said two or more alleles are alleles from two or more different correlated clusters selected from those provided in Table 7.

In still a further embodiment, a method is provided for detecting the presence of a polymorphism in and administering a treatment to a human subject, the method comprising (a) obtaining a genomic sample from a human subject having or at risk of developing SZ; (b) detecting the haplotype tagged by an allele selected from those provided in Table 2B in the genomic sample; (c) identifying the subject having the haplotype tagged by the allele provided in Table 2B in the genomic sample as likely to have a poor response to perphenazine as compared to control subject; and (d) administering an antipsychotic treatment other than perphenazine to the subject with the haplotype tagged by the allele provided in Table 2B. In certain aspects, the method further comprises detecting the haplotype tagged by two or more alleles (e.g., 1, 2, 3, 4, 5, 6, 7, 8, 9 or more alleles) selected from those provided in Table 2B. In one aspect, said two or more alleles are alleles from two or more different correlated clusters selected from those provided in Table 7. In certain aspects, the method comprises administering olanzapine, quetiapine, risperidone or ziprasidone to the subject.

Thus, in some aspects, a method is provided for detecting the presence of a polymorphism in the MCPH1, PRKCE, CDH13, or SKOR2 gene and administering a treatment to a human subject, the method comprising (a) obtaining a genomic sample from a human subject having or at risk of developing SZ; (b) detecting the haplotype tagged by the “C” allele of rs11774231, the haplotype tagged by the “C” allele of rs2278773, the haplotype tagged by the “A” allele of rs17570753, the haplotype tagged by the “C” allele of rs2116971, or the haplotype tagged by the “G” allele of rs9952628 in the genomic sample; (c) identifying the subject having the haplotype tagged by the “C” allele of rs11774231, the haplotype tagged by the “C” allele of rs2278773, the haplotype tagged by the “A” allele of rs17570753, the haplotype tagged by the “C” allele of rs2116971, or the haplotype tagged by the “G” allele of rs9952628 in the genomic sample as likely to have an improved response to perphenazine as compared to control subject; and (d) administering a treatment comprising perphenazine to the subject with the haplotype tagged by the “C” allele of rs11774231, the haplotype tagged by the “C” allele of rs2278773, the haplotype tagged by the “A” allele of rs17570753, the haplotype tagged by the “C” allele of rs2116971, or the haplotype tagged by the “G” allele of rs9952628.

In further aspects, a method is provided for detecting the presence of a polymorphism in the MAML3 gene and administering a treatment to a human subject, the method comprising (a) obtaining a genomic sample from a human subject having or at risk of developing SZ; (b) detecting the haplotype tagged by the “A” allele of rs11100483 in the genomic sample; (c) identifying the subject having the haplotype tagged by the “A” allele of rs11100483 in the genomic sample as likely to have a poor response to perphenazine as compared to control subject; and (d) administering an antipsychotic treatment other than perphenazine to the subject with the haplotype tagged by the “A” allele of rs11100483. In certain aspects, the method comprises administering olanzapine, quetiapine, risperidone or ziprasidone to the subject.

In a further embodiment, the present invention provides a method of detecting the presence of a polymorphism in and administering a treatment to a human subject, the method comprising (a) obtaining a genomic sample from a human subject having or at risk of developing SZ; (b) detecting the haplotype tagged by an allele selected from those provided in Table 3A in the genomic sample; (c) identifying the subject having the haplotype tagged by the allele provided in Table 3A in the genomic sample as likely to have an improved response to quetiapine as compared to control subject; and (d) administering a treatment comprising quetiapine to the subject with the haplotype tagged by the allele provided in Table 3A. In certain aspects, the method further comprises detecting the haplotype tagged by two or more alleles (e.g., 1, 2, 3, 4, 5, 6, 7, 8, 9 or more alleles) selected from those provided in Table 3A. In one aspect, said two or more alleles are alleles from two or more different correlated clusters selected from those provided in Table 8.

In yet a further embodiment, the present invention provides a method of detecting the presence of a polymorphism in and administering a treatment to a human subject, the method comprising (a) obtaining a genomic sample from a human subject having or at risk of developing SZ; (b) detecting the haplotype tagged by an allele selected from those provided in Table 3B in the genomic sample; (c) identifying the subject having the haplotype tagged by the allele provided in Table 3B in the genomic sample as likely to have a poor response to quetiapine as compared to control subject; and (d) administering an antipsychotic treatment other than quetiapine to the subject with the haplotype tagged by the allele provided in Table 3B. In certain aspects, the method further comprises detecting the haplotype tagged by two or more alleles (e.g., 1, 2, 3, 4, 5, 6, 7, 8, 9 or more alleles) selected from those provided in Table 3B. In one aspect, said two or more alleles are alleles from two or more different correlated clusters selected from those provided in Table 8. In certain aspects, the method comprises administering olanzapine, perphenazine, risperidone or ziprasidone to the subject.

In some aspects a method is provided for detecting the presence of a polymorphism in the KCNMA1 gene and administering a treatment to a human subject, the method comprising (a) obtaining a genomic sample from a human subject having or at risk of developing SZ; (b) detecting the haplotype tagged by the “C” allele of rs35793; (c) identifying the subject having the haplotype tagged by the “C” allele of rs35793 in the genomic sample as likely to have a poor response to quetiapine as compared to control subject; and (d) administering an antipsychotic treatment other than quetiapine to the subject with the haplotype tagged by the “C” allele of rs35793. In certain aspects, the method comprises administering olanzapine, perphenazine, risperidone or ziprasidone to the subject.

In a further embodiment, the present invention provides a method of detecting the presence of a polymorphism in and administering a treatment to a human subject, the method comprising (a) obtaining a genomic sample from a human subject having or at risk of developing SZ; (b) detecting the haplotype tagged by an allele selected from those provided in Table 4A in the genomic sample; (c) identifying the subject having the haplotype tagged by the allele provided in Table 4A in the genomic sample as likely to have an improved response to risperidone as compared to control subject; and (d) administering a treatment comprising risperidone to the subject with the haplotype tagged by the allele provided in Table 4A. In certain aspects, the method further comprises detecting the haplotype tagged by two or more alleles (e.g., 1, 2, 3, 4, 5, 6, 7, 8, 9 or more alleles) selected from those provided in Table 4A. In one aspect, said two or more alleles are alleles from two or more different correlated clusters selected from those provided in Table 9.

In still a further embodiment, the present invention provides a method of detecting the presence of a polymorphism in and administering a treatment to a human subject, the method comprising (a) obtaining a genomic sample from a human subject having or at risk of developing SZ; (b) detecting the haplotype tagged by an allele selected from those provided in Table 4B in the genomic sample; (c) identifying the subject having the haplotype tagged by the allele provided in Table 4B in the genomic sample as likely to have a poor response to risperidone as compared to control subject; and (d) administering an antipsychotic treatment other than risperidone to the subject with the haplotype tagged by the allele provided in Table 4B. In certain aspects, the method further comprises detecting the haplotype tagged by two or more alleles (e.g., 1, 2, 3, 4, 5, 6, 7, 8, 9 or more alleles) selected from those provided in Table 4B. In one aspect, said two or more alleles are alleles from two or more different correlated clusters selected from those provided in Table 9. In certain aspects, the method comprises administering olanzapine, perphenazine, quetiapine or ziprasidone to the subject.

Thus, in some aspects a method is provided for detecting the presence of a polymorphism in the PSMD14, LRP1B, or TMEFF2 gene and administering a treatment to a human subject, the method comprising (a) obtaining a genomic sample from a human subject having or at risk of developing SZ; (b) detecting the haplotype tagged by the “A” allele of rs9713, the haplotype tagged by the “C” allele of rs874295, or the haplotype tagged by the “C” allele of rs3738883 in the genomic sample; (c) identifying the subject having the haplotype tagged by the “A” allele of rs9713, the haplotype tagged by the “C” allele of rs874295, or the haplotype tagged by the “C” allele of rs3738883 in the genomic sample as likely to have an improved response to risperidone as compared to control subject; and (d) administering a treatment comprising risperidone to the subject with the haplotype tagged by the “A” allele of rs9713, the haplotype tagged by the “C” allele of rs874295, or the haplotype tagged by the “C” allele of rs3738883.

In further aspects a method is provided for detecting the presence of a polymorphism in the AGAP1 or NPAS3 gene and administering a treatment to a human subject, the method comprising (a) obtaining a genomic sample from a human subject having or at risk of developing SZ; (b) detecting the haplotype tagged by the “C” allele of rs1869295 or the haplotype tagged by the “C” allele of rs1315115 in the genomic sample; (c) identifying the subject having the haplotype tagged by the “C” allele of rs1869295 or the haplotype tagged by the “C” allele of rs1315115 in the genomic sample as likely to have a poor response to risperidone as compared to control subject; and (d) administering an antipsychotic treatment other than risperidone to the subject with the haplotype tagged by the “C” allele of rs1869295 or the haplotype tagged by the “C” allele of rs1315115. In certain aspects, the method comprises administering olanzapine, perphenazine, quetiapine or ziprasidone to the subject.

In still a further embodiment, the present invention provides a method of detecting the presence of a polymorphism in and administering a treatment to a human subject, the method comprising (a) obtaining a genomic sample from a human subject having or at risk of developing SZ; (b) detecting the haplotype tagged by an allele selected from those provided in Table 5A in the genomic sample; (c) identifying the subject having the haplotype tagged by the allele provided in Table 5A in the genomic sample as likely to have an improved response to ziprasidone as compared to control subject; and (d) administering a treatment comprising ziprasidone to the subject with the haplotype tagged by the allele provided in Table 5A. In certain aspect, the method further comprises detecting the haplotype tagged by two or more alleles (e.g., 1, 2, 3, 4, 5, 6, 7, 8, 9 or more alleles) selected from those provided in Table 5A. In one aspect, said two or more alleles are alleles from two or more different correlated clusters selected from those provided in Table 10.

In yet a further embodiment, the present invention provides a method of detecting the presence of a polymorphism in and administering a treatment to a human subject, the method comprising (a) obtaining a genomic sample from a human subject having or at risk of developing SZ; (b) detecting the haplotype tagged by an allele selected from those provided in Table 5B in the genomic sample; (c) identifying the subject having the haplotype tagged by the allele provided in Table 5B in the genomic sample as likely to have a poor response to ziprasidone as compared to control subject; and (d) administering an antipsychotic treatment other than ziprasidone to the subject with the haplotype tagged by the allele provided in Table 5B. In certain aspects, the method further comprises detecting the haplotype tagged by two or more alleles (e.g., 1, 2, 3, 4, 5, 6, 7, 8, 9 or more alleles) selected from those provided in Table 5B. In one aspect, said two or more alleles are alleles from two or more different correlated clusters selected from those provided in Table 10. In certain aspects, the method comprises administering olanzapine, perphenazine, quetiapine or risperidone to the subject.

Thus, in some aspects, a method is provided for detecting the presence of a polymorphism in the CDH4, LYN, or CNTN4 gene and administering a treatment to a human subject, the method comprising (a) obtaining a genomic sample from a human subject having or at risk of developing SZ; (b) detecting the haplotype tagged by the “A” allele of rs4925300, the haplotype tagged by the “C” allele of rs1546519, or the haplotype tagged by the “A” allele of rs17194378 in the genomic sample; (c) identifying the subject having the haplotype tagged by the “A” allele of rs4925300, the haplotype tagged by the “C” allele of rs1546519, or the haplotype tagged by the “A” allele of rs17194378 in the genomic sample as likely to have an improved response to ziprasidone as compared to control subject; and (d) administering a treatment comprising ziprasidone to the subject with the haplotype tagged by the “A” allele of rs4925300, the haplotype tagged by the “C” allele of rs1546519, or the haplotype tagged by the “A” allele of rs17194378.

In some aspects, the present invention provides a method of detecting the presence of a polymorphism in the NALCN gene and administering a treatment to a human subject, the method comprising (a) obtaining a genomic sample from a human subject having or at risk of developing SZ; (b) detecting the haplotype tagged by the “C” allele of rs9585618 in the genomic sample; (c) identifying the subject having the haplotype tagged by the “C” allele of rs9585618 in the genomic sample as likely to have a poor response to ziprasidone as compared to control subject; and (d) administering an antipsychotic treatment other than ziprasidone to the subject with the haplotype tagged by the “C” allele of rs9585618. In certain aspects, the method comprises administering olanzapine, perphenazine, quetiapine or risperidone to the subject.

In certain aspects of the present embodiments, the subject may have early, intermediate, or aggressive SZ. In certain aspects of the present embodiments, the subject may have one or more risk factors associated with SZ. In certain aspects of the present embodiments, the subject may have a relative afflicted with SZ or a genetically-based phenotypic trait associated with risk for SZ. In certain aspects of the present embodiments, the subject may be Caucasian or comprise European ancestry. In certain aspects of the present embodiments, determining the haplotype tagged by an allele may comprise determining the number of alleles tagging the haplotype in the subject.

In still a further embodiment, the present invention provides a method of identifying and administering a treatment to a human subject, the method comprising (a) obtaining a genomic sample from a human subject having or at risk of developing SZ; (b) detecting two or more haplotypes tagged by an allele selected from those provided in Table 1 for olanzapine, Table 2, for perphenazine, Table 3 for quetiapine, Table 4 for risperidone, or Table 5 for ziprasidone in the genomic sample; (c) calculating a predicted treatment efficacy for at least two drugs selected from the group consisting of olanzapine, perphenazine, quetiapine, risperidone, and ziprasidone; (d) ranking the predicted efficacy of the at least two drugs; and (e) administering a treatment to the subject based on said ranking. In one aspect, detecting two or more haplotypes tagged by an allele comprises determining the number of alleles tagging the two or more haplotypes in the subject. In further aspects, calculating a predicted treatment efficacy for a given drug comprises assigning a weighted value to each haplotype and multiplying the weighted value by the number of alleles tagging the haplotype in the subject. In another aspect, calculating a predicted treatment efficacy comprises using the equation:

P=C+ΣᵢβᵢNᵢ

wherein P is the predicted treatment efficacy measured in change in PANSS-T; C is the change in PANSS-T for individuals carrying zero alleles of any response-predicting haplotype for the drug, β is the weighted value for at least a first haplotype measured in PANSS-T; N is the number of alleles tagging at least the first haplotype; and i is the number of haplotypes detected. In one aspect, the method comprises determining a predicted treatment efficacy for three, four or five drugs selected from the group consisting of olanzapine, perphenazine, quetiapine, risperidone, and ziprasidone. In certain aspects, the subject may have early, intermediate, or aggressive SZ. In certain aspects, the subject may have one or more risk factors associated with SZ. In certain aspects, the subject may have a relative afflicted with SZ or a genetically-based phenotypic trait associated with risk for SZ. In certain aspects, the subject may by Caucasian or comprise European ancestry.

In aspect of the invention involving determining whether genetic material of the subject comprises a haplotype, the need transfer and store genetic information will be preferably met by recording and maintaining the information in a tangible medium, such as a computer-readable disk, a solid state memory device, an optical storage device or the like, more specifically, a storage device such as a hard drive, a Compact Disk (CD) drive, a floppy disk drive, a tape drive, a random access memory (RAM), etc.

One preferred manner of obtaining the haplotype information involves analyzing the genetic material of the subject to determine the presence or absence of the haplotype. This can be accomplished, for example, by testing the subject's genetic material through the use of a biological sample. In certain embodiments, the methods set forth will thus involve obtaining a biological sample from the subject and testing the biological sample to identify whether an haplotype is present. The biological sample may be any biological material that contains DNA or RNA of the subject, such as a nucleated cell source. Non-limiting examples of cell sources available in clinical practice include hair, skin, nucleated blood cells, buccal cells, any cells present in tissue obtained by biopsy or any other cell collection method. The biological sample may also be obtained from body fluids, including without limitation blood, saliva, sweat, urine, amniotic fluid (the fluid that surrounds a fetus during pregnancy), cerebrospinal fluid, feces, and tissue exudates at the site of infection or inflammation. DNA may be extracted from the biologic sample such as the cell source or body fluid using any of the numerous methods that are standard in the art.

Determining whether the genetic material exhibits an haplotype can be by any method known to those of ordinary skill in the art, such as genotyping (e.g., SNP genotyping) or sequencing. Techniques that may be involved in this determination are well-known to those of ordinary skill in the art. Examples of such techniques include allele specific oligonucleotide hybridization, size analysis, sequencing, hybridization, 5′ nuclease digestion, single-stranded conformation polymorphism analysis, allele specific hybridization, primer specific extension, and oligonucleotide ligation assays. Additional information regarding these techniques is discussed in the specification below.

For haplotype determinations, the sequence of the extracted nucleic acid of the subject may be determined by any means known in the art, including but not limited to direct sequencing, hybridization with allele-specific oligonucleotides, allele-specific PCR, ligase-PCR, HOT cleavage, denaturing gradient gel electrophoresis (DDGE), and single-stranded conformational polymorphism (SSCP) analysis. Direct sequencing may be accomplished by any method, including without limitation chemical sequencing, using the Maxam-Gilbert method, by enzymatic sequencing, using the Sanger method; mass spectrometry sequencing; and sequencing using a chip-based technology. In particular embodiments, DNA from a subject is first subjected to amplification by polymerase chain reaction (PCR) using specific amplification primers. In some embodiments, the method further involves amplification of a nucleic acid from the biological sample. The amplification may or may not involve PCR. In some embodiments, the primers are located on a chip.

Moreover, the inventors contemplate that the genetic structure and sequence, including SNP profiles, of individual subjects will at some point be widely or generally available, or will have been developed by an unrelated third party. In such instances, there will be no need to test or analyze the subject's biological material again. Instead, the genetic information will in such cases be obtained simply by analyzing the sequencing or genotyping outcome of the subject, for example, a SNP profile, a whole or partial genome sequence, etc. These outcomes can then be obtained from or reported by a sequencing or a genotyping service, a laboratory, a scientist, or any genetic test platforms.

In some further aspects, the method may further comprise reporting the determination to the subject, a health care payer, an attending clinician, a pharmacist, a pharmacy benefits manager, or any person that the determination may be of interest.

Any of the SNPs listed in Tables 1-10 can be readily mapped on to the publically available human genome sequence (e.g., NCBI Human Genome Build 37.3). For each of the SNPs listed herein the reference SNP (rs) number is provided, which provides the known sequence context for the given SNP (see, e.g., National Center for Biotechnology Information (NCBI) SNP database available on the world wide web at ncbi.nlm.nih.gov/snp).

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Summary of functional categories of newly evaluated SNPs on the custom BeadChip, based on NCBI resources.

FIG. 2: Q-Q (quantile-quantile) plots for log₁₀ transformed observed P values from the association tests using the MMRM-predicted change in PANSS-T for olanzapine, perphenazine, quetiapine, risperidone, and ziprasidone. This analysis is limited to SNPs with minor allele frequencies ≧0.03 in the particular drug arm. Gray areas represent 95% confidence intervals. If the slope for observed P values (blue circles) is steeper than the baseline assumption (red line, y=x), overall the observed P values are more significant than P values expected based on a theoretical distribution.

### DESCRIPTION OF ILLUSTRATIVE EMBODIMENTS

Studies detailed herein identify over 6,000 SNPs in genes impacting disease risk, disease presentation, and, particularly, response to antipsychotics drug treatment. Most of the SNPs tag regions of linkage disequilibrium or represent functional variants that could not have been detected using the original genotypes provided by the CATIE consortium. Association analyses using the mixed model repeated measures approach of van den Oord and coworkers (van den Oord et al., 2009; McClay et al., 2011) identified numerous SNPs predicting response to olanzapine, perphenazine, quetiapine, risperidone, and ziprasidone.

The targeted genotyping approach described here resulted in numerous associations for SNPs impacting response to antipsychotic medications for treatment of schizophrenia. The association results are not biased by post hoc selection of a response variable, due to the fact that a previously published MMRM-based approach was used to measure treatment response (van den Oord et al., 2009). Tables 1-5 provide association results for all 6,789 newly genotyped SNPs with nominal P values <0.05. Included in these tables are numerous examples of individual SNPs that impact response to one or more antipsychotic drugs.

Of the genes with the most significant SNP associations, only NPAS3 has been reported previously to contain common genetic variation that impacts response to antipsychotic treatment of schizophrenia. In the present study, rs1315115, located in an intron of NPAS3, was associated with response to risperidone. Lavedan and coworkers reported the association of SNPs in NPAS3 with response to the structurally related drug iloperidone (Lavedan et al., 2009).

In the present studies, analysis was limited to the Caucasian patients to minimize effects of population stratification, in contrast to most previous studies, which combined subpopulations and used principal component adjustment for population stratification to increase sample size (McClay et al., 2011; Sullivan et al., 2008). Further, chromosomal regions not previously evaluated for the CATIE sample were targeted to generate results that would complement rather than replicate previous findings for CATIE. The findings of these studies follow the pattern seen by others in that no single SNP was associated strongly with response to more than one drug (McClay et al., 2011; Need et al., 2009). This is not surprising considering the diverse mechanisms of action for the various antipsychotic drugs evaluated in the CATIE study (Meltzer et al., 2008).

The custom Illumina iSelect BeadChip was designed to capture common genetic variation, including functional variation, in genes suspected of having an impact on disease presentation or response to antipsychotics. As expected based on the linkage disequilibrium (LD) information available at the time the BeadChip was designed, most of the SNPs defined, as well as tagged, haplotype blocks that could not have been detected using only SNP genotypes provided by the CATIE group.

### I. DEFINITIONS

As used herein, an “allele” is one of a pair or series of genetic variants of a polymorphism at a specific genomic location. A “response allele” is an allele that is associated with altered response to a treatment. Where a SNP is biallelic, both alleles will be response alleles (e.g., one will be associated with a positive response, while the other allele is associated with no or a negative response, or some variation thereof).

As used herein, “genotype” refers to the diploid combination of alleles for a given genetic polymorphism. A homozygous subject carries two copies of the same allele and a heterozygous subject carries two different alleles.

As used herein, a “haplotype” is one or a set of signature genetic changes (polymorphisms) that are normally grouped closely together on the DNA strand, and are inherited as a group; the polymorphisms are also referred to herein as “markers.” A “haplotype” as used herein is information regarding the presence or absence of one or more genetic markers in a given chromosomal region in a subject. A haplotype can consist of a variety of genetic markers, including indels (insertions or deletions of the DNA at particular locations on the chromosome); single nucleotide polymorphisms (SNPs) in which a particular nucleotide is changed; microsatellites; and minisatellites.

Microsatellites (sometimes referred to as a variable number of tandem repeats or VNTRs) are short segments of DNA that have a repeated sequence, usually about 2 to 5 nucleotides long (e.g., a CA nucleotide pair repeated three times), that tend to occur in non-coding DNA. Changes in the microsatellites sometimes occur during the genetic recombination of sexual reproduction, increasing or decreasing the number of repeats found at an allele, changing the length of the allele. Microsatellite markers are stable, polymorphic, easily analyzed and occur regularly throughout the genome, making them especially suitable for genetic analysis.

“Copy number variation” (CNV), as used herein, refers to variation from the normal diploid condition for a gene or polymorphism. Individual segments of human chromosomes can be deleted or duplicated such that the subject's two chromosomes carry fewer than two copies of the gene or polymorphism (a deletion or deficiency) or two or more copies (a duplication).

“Linkage disequilibrium” (LD) refers to when the observed frequencies of haplotypes in a population does not agree with haplotype frequencies predicted by multiplying together the frequency of individual genetic markers in each haplotype. When SNPs and other variations that comprise a given haplotype are in LD with one another, alleles at the different markers correlate with one another.

The term “chromosome” as used herein refers to a gene carrier of a cell that is derived from chromatin and comprises DNA and protein components (e.g., histones). The conventional internationally recognized individual human genome chromosome numbering identification system is employed herein. The size of an individual chromosome can vary from one type to another with a given multi-chromosomal genome and from one genome to another. In the case of the human genome, the entire DNA mass of a given chromosome is usually greater than about 100,000,000 base pairs. For example, the size of the entire human genome is about 3×10⁹ base pairs.

The term “gene” refers to a DNA sequence in a chromosome that codes for a product (either RNA or its translation product, a polypeptide). A gene contains a coding region and includes regions preceding and following the coding region (termed respectively “leader” and “trailer”). The coding region is comprised of a plurality of coding segments (“exons”) and intervening sequences (“introns”) between individual coding segments.

The term “probe” refers to an oligonucleotide. A probe can be single stranded at the time of hybridization to a target. As used herein, probes include primers, i.e., oligonucleotides that can be used to prime a reaction, e.g., a PCR reaction.

The term “label” or “label containing moiety” refers in a moiety capable of detection, such as a radioactive isotope or group containing the same, and nonisotopic labels, such as enzymes, biotin, avidin, streptavidin, digoxygenin, luminescent agents, dyes, haptens, and the like. Luminescent agents, depending upon the source of exciting energy, can be classified as radioluminescent, chemiluminescent, bioluminescent, and photoluminescent (including fluorescent and phosphorescent). A probe described herein can be bound, e.g., chemically bound to label-containing moieties or can be suitable to be so bound. The probe can be directly or indirectly labeled.

The term “direct label probe” (or “directly labeled probe”) refers to a nucleic acid probe whose label after hybrid formation with a target is detectable without further reactive processing of the hybrid. The term “indirect label probe” (or “indirectly labeled probe”) refers to a nucleic acid probe whose label after hybrid formation with a target is further reacted in subsequent processing with one or more reagents to associate therewith one or more moieties that finally result in a detectable entity.

The terms “target,” “DNA target,” or “DNA target region” refers to a nucleotide sequence that occurs at a specific chromosomal location. Each such sequence or portion is preferably, at least partially, single stranded (e.g., denatured) at the time of hybridization. When the target nucleotide sequences are located only in a single region or fraction of a given chromosome, the term “target region” is sometimes used. Targets for hybridization can be derived from specimens that include, but are not limited to, chromosomes or regions of chromosomes in normal, diseased or malignant human cells, either interphase or at any state of meiosis or mitosis, and either extracted or derived from living or postmortem tissues, organs or fluids; germinal cells including sperm and egg cells, or cells from zygotes, fetuses, or embryos, or chorionic or amniotic cells, or cells from any other germinating body; cells grown in vitro, from either long-term or short-term culture, and either normal, immortalized or transformed; inter- or intraspecific hybrids of different types of cells or differentiation states of these cells; individual chromosomes or portions of chromosomes, or translocated, deleted or other damaged chromosomes, isolated by any of a number of means known to those with skill in the art, including libraries of such chromosomes cloned and propagated in prokaryotic or other cloning vectors, or amplified in vitro by means well known to those with skill; or any forensic material, including but not limited to blood, or other samples.

The term “hybrid” refers to the product of a hybridization procedure between a probe and a target.

The term “hybridizing conditions” has general reference to the combinations of conditions that are employable in a given hybridization procedure to produce hybrids, such conditions typically involving controlled temperature, liquid phase, and contact between a probe (or probe composition) and a target. Conveniently and preferably, at least one denaturation step precedes a step wherein a probe or probe composition is contacted with a target. Guidance for performing hybridization reactions can be found in Ausubel et al., Current Protocols in Molecular Biology, John Wiley & Sons, N.Y. (2003), 6.3.1-6.3.6. Aqueous and nonaqueous methods are described in that reference and either can be used. Hybridization conditions referred to herein are a 50% formamide, 2×SSC wash for 10 minutes at 45° C. followed by a 2×SSC wash for 10 minutes at 37° C.

Calculations of “identity” between two sequences can be performed as follows. The sequences are aligned for optimal comparison purposes (e.g., gaps can be introduced in one or both of a first and a second nucleic acid sequence for optimal alignment and non-identical sequences can be disregarded for comparison purposes). The length of a sequence aligned for comparison purposes is at least 30% (e.g., at least 40%, 50%, 60%, 70%, 80%, 90% or 100%) of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In some embodiments, the percent identity between two nucleotide sequences is determined using the GAP program in the GCG software package, using a Blossum 62 scoring matrix with a gap penalty of 12, a gap extend penalty of 4, and a frameshift gap penalty of 5.

As used herein, the term “substantially identical” is used to refer to a first nucleotide sequence that contains a sufficient number of identical nucleotides to a second nucleotide sequence such that the first and second nucleotide sequences have similar activities. Nucleotide sequences that are substantially identical are at least 80% (e.g., 85%, 90%, 95%, 97% or more) identical.

The term “nonspecific binding DNA” refers to DNA that is complementary to DNA segments of a probe, which DNA occurs in at least one other position in a genome, outside of a selected chromosomal target region within that genome. An example of nonspecific binding DNA comprises a class of DNA repeated segments whose members commonly occur in more than one chromosome or chromosome region. Such common repetitive segments tend to hybridize to a greater extent than other DNA segments that are present in probe composition.

As used herein, the term “stratification” refers to the creation of a distinction between subjects on the basis of a characteristic or characteristics of the subjects. Generally, in the context of clinical trials, the distinction is used to distinguish responses or effects in different sets of patients distinguished according to the stratification parameters. In some embodiments, stratification includes distinction of subject groups based on the presence or absence of particular markers or alleles described herein. The stratification can be performed, e.g., in the course of analysis, or can be used in creation of distinct groups or in other ways.

### II. METHODS OF PREDICTING RESPONSE AND SELECTING OPTIMAL TREATMENT

Described herein are a variety of methods for predicting a subject's response, or selecting and optimizing (and optionally administering) a treatment for a subject having an SSD (e.g., SZ) based on the presence or absence of a response allele.

As used herein, “determining the identity of an allele” includes obtaining information regarding the identity (i.e., of a specific nucleotide), presence or absence of one or more specific alleles in a subject. Determining the identity of an allele can, but need not, include obtaining a sample comprising DNA from a subject, and/or assessing the identity, presence or absence of one or more genetic markers in the sample. The individual or organization who determines the identity of the allele need not actually carry out the physical analysis of a sample from a subject; the methods can include using information obtained by analysis of the sample by a third party. Thus the methods can include steps that occur at more than one site. For example, a sample can be obtained from a subject at a first site, such as at a health care provider, or at the subject's home in the case of a self-testing kit. The sample can be analyzed at the same or a second site, e.g., at a laboratory or other testing facility.

Determining the identity of an allele can also include or consist of reviewing a subject's medical history, where the medical history includes information regarding the identity, presence or absence of one or more response alleles in the subject, e.g., results of a genetic test.

In some embodiments, to determine the identity of an allele described herein, a biological sample that includes nucleated cells (such as blood, a cheek swab or mouthwash) is prepared and analyzed for the presence or absence of preselected markers. Such diagnoses may be performed by diagnostic laboratories, or, alternatively, diagnostic kits can be manufactured and sold to health care providers or to private individuals for self-diagnosis. Diagnostic or prognostic tests can be performed as described herein or using well known techniques, such as described in U.S. Pat. No. 5,800,998.

Results of these tests, and optionally interpretive information, can be returned to the subject, the health care provider or to a third party payor. The results can be used in a number of ways. The information can be, e.g., communicated to the tested subject, e.g., with a prognosis and optionally interpretive materials that help the subject understand the test results and prognosis. The information can be used, e.g., by a health care provider, to determine whether to administer a specific drug, or whether a subject should be assigned to a specific category, e.g., a category associated with a specific disease endophenotype, or with drug response or non-response. The information can be used, e.g., by a third party payor such as a healthcare payer (e.g., insurance company or HMO) or other agency, to determine whether or not to reimburse a health care provider for services to the subject, or whether to approve the provision of services to the subject. For example, the healthcare payer may decide to reimburse a health care provider for treatments for an SSD if the subject has a particular response allele. As another example, a drug or treatment may be indicated for individuals with a certain allele, and the insurance company would only reimburse the health care provider (or the insured individual) for prescription or purchase of the drug if the insured individual has that response allele. The presence or absence of the response allele in a patient may be ascertained by using any of the methods described herein.

### A. Response Alleles

This document provides methods for predicting response and selecting an optimal treatment based on evaluation of one or more single nucleotide polymorphisms (SNPs) associated with specific treatment responses in subjects having SZ or SZ-spectrum disorders including SZ, SPD, or SD. Table A and Tables 1-5 list specific SNPs, variation of which is associated with altered response to treatment. One of skill in the art will appreciate that other variants can be identified and verified by Case/Control comparisons using the SNP markers presented herein. Using SNP markers that are identical to or in linkage disequilibrium with the exemplary SNPs, one can determine additional alleles of the genes, such as haplotypes, relating to response to treatment of an SSD (e.g., SZ). The allelic variants thus identified can be used, e.g., to select optimal treatments (e.g., pharmaceutical and/or psychosocial intervention) for patients.

TABLE A
Summary of SNPs (NCBI Human Genome Build 37.3)

| Gene | Chr | Position (BP) | Gene | Chr | Position (BP) |
|---|---|---|---|---|---|
| CSMD1 | 8 | 4,467,528 | PLAGL1; | 6 | 144,278,262 |
| | | | LOC100652728 | | |
| PLAGL1; | 6 | 144,280,529 | PTPRN2 | 7 | 157,332,172 |
| LOC100652728 | | | | | |
| KIAA0182 | 16 | 85,700,360 | FREM1 | 9 | 14,801,738 |
| CNTNAP2 | 7 | 147,750,195 | ZNF71 | 19 | 57,105,726 |
| PCDH15 | 10 | 55,629,421 | DGKD | 2 | 234,296,650 |
| PDGFD; DDI1 | 11 | 103,908,968 | SEMA5A | 5 | 9,445,434 |
| ROBO2 | 3 | 77,103,671 | CNTN4 | 3 | 2,287,920 |
| CNTN4 | 3 | 2,294,001 | NAB1 | 2 | 191,520,845 |
| GRIN3A | 9 | 104,470,105 | GPC6 | 13 | 94,021,687 |
| DLG2 | 11 | 84,529,337 | DLG2 | 11 | 84,434,238 |
| PARK2 | 6 | 161,981,577 | DLG2 | 11 | 84,467,936 |
| SCN3A | 2 | 165,996,327 | SEMA5A | 5 | 9,034,674 |
| IL17RD | 3 | 57,125,380 | TPH2 | 12 | 72,430,314 |
| RAP1GAP2 | 17 | 2,940,720 | GRIK3 | 1 | 37,186,174 |
| SEMA3F | 3 | 50,222,926 | RBFOX1 | 16 | 6,029,562 |
| PCDH15 | 10 | 56,309,093 | CA10 | 17 | 49,868,887 |
| NPAS3 | 14 | 33,608,871 | CSMD1 | 8 | 4,493,580 |
| MAGI2 | 7 | 77,917,038 | AK5 | 1 | 77,828,321 |
| SLC16A9 | 10 | 61,431,755 | FAM19A1 | 3 | 68,197,782 |
| CSMD1 | 8 | 4,812,290 | PKNOX2 | 11 | 125,301,805 |
| PSD3 | 8 | 18,933,166 | PPA2 | 4 | 106,364,129 |
| CLIC5 | 6 | 45,916,999 | PTGER3 | 1 | 71,476,872 |
| CNTNAP2 | 7 | 146,198,525 | NELL1 | 11 | 21,418,874 |
| DLG2 | 11 | 84,457,943 | KITLG | 12 | 88,890,521 |
| FBN3 | 19 | 8,130,420 | LIMCH1 | 4 | 41,658,985 |
| CNTNAP2 | 7 | 147,725,495 | DENND5B | 12 | 31,536,772 |
| DLGAP2 | 8 | 1,649,938 | UNC13C | 15 | 54,891,716 |
| COL22A1 | 8 | 139,666,447 | HPS4 | 22 | 26,877,463 |
| SYN3 | 22 | 32,908,723 | ERBB4 | 2 | 212,946,149 |
| WNT5B | 12 | 1,753,844 | ANO2 | 12 | 5,940,753 |
| ADTRP | 6 | 11,774,792 | PCLO | 7 | 82,730,446 |
| PCDH15 | 10 | 55,747,770 | CNTNAP2 | 7 | 146,202,073 |
| PPARG | 3 | 12,500,651 | LOC100129345 | 14 | 98,112,541 |
| CNTNAP2 | 7 | 147,607,367 | JPH2 | 20 | 42,805,719 |
| CNTNAP2 | 7 | 147,958,959 | CHN2 | 7 | 29,212,051 |
| CSMD1 | 8 | 4,493,912 | PLD1 | 3 | 171,324,666 |
| CDH23 | 10 | 73,457,735 | ANK3 | 10 | 61,800,837 |
| NOS1AP | 1 | 162,339,754 | ANK3 | 10 | 62,088,266 |
| ANK3 | 10 | 62,105,916 | GALNTL4 | 11 | 11,521,053 |
| NAB1 | 2 | 191,510,532 | NALCN | 13 | 101,801,061 |
| VDAC1 | 5 | 133,326,423 | CNTNAP2 | 7 | 146,848,333 |
| ELOVL7 | 5 | 60,049,577 | DLG2 | 11 | 83,166,720 |
| CACNA2D3 | 3 | 54,949,461 | MTIF3 | 13 | 28,022,617 |
| INS-IGF2; IGF2 | 11 | 2,157,044 | LYN | 8 | 56,808,662 |
| CDH20 | 18 | 59,218,968 | CGNL1 | 15 | 57,806,751 |
| TSNAX-DISC1 | 1 | 232,177,487 | CNTNAP2 | 7 | 146,833,030 |
| DGKD | 2 | 234,296,444 | CPNE4 | 3 | 131,659,901 |
| NTNG2 | 9 | 135,110,456 | SCD5 | 4 | 83,722,608 |
| ZNF804A | 2 | 185,524,642 | TMC8 | 17 | 76,137,337 |
| CSMD1 | 8 | 2,809,544 | KCNJ2 | 17 | 69,089,167 |
| OPCML | 11 | 133,055,664 | DLG2 | 11 | 84,456,685 |
| GRIN3A | 9 | 104,469,267 | SLC1A3 | 5 | 36,648,442 |
| WDR48 | 3 | 39,137,883 | HPCAL1 | 2 | 10,502,982 |
| KLHL32 | 6 | 97,374,850 | WWC1 | 5 | 167,872,381 |
| KCNN2 | 5 | 113,832,673 | IFT74 | 9 | 26,953,102 |
| IFT74 | 9 | 26,955,661 | FMN2 | 1 | 240,301,487 |
| ARVCF | 22 | 19,996,878 | FBN3 | 19 | 8,203,113 |
| KCNQ1 | 11 | 2,473,131 | MAGI2 | 7 | 78,890,598 |
| BRE; | 2 | 28,531,903 | CNTN4 | 3 | 2,342,825 |
| LOC100505716 | | | | | |
| GRIA1 | 5 | 152,908,929 | CSMD1 | 8 | 3,513,875 |
| NALCN | 13 | 101,843,483 | CSMD3 | 8 | 113,235,729 |
| CNTN4 | 3 | 2,152,057 | GPSM1 | 9 | 139,251,945 |
| SLC25A18 | 22 | 18,073,592 | KIAA1797 | 9 | 20,740,671 |
| CTNNA3 | 10 | 67,679,568 | ITPR1 | 3 | 4,685,191 |
| CDH13 | 16 | 82,778,559 | FAM46C | 1 | 118,167,653 |
| SYNPR | 3 | 63,437,186 | TRPM3 | 9 | 73,564,717 |
| IGF2R | 6 | 160,448,324 | GRB10 | 7 | 50,675,930 |
| PRICKLE2 | 3 | 64,106,014 | SLC35F3 | 1 | 234,166,807 |
| SLIT1 | 10 | 98,875,408 | PLCB1 | 20 | 8,317,335 |
| C9orf84 | 9 | 114,454,544 | CACNA2D3 | 3 | 54,948,993 |
| PHACTR3 | 20 | 58,160,628 | NCAM2 | 21 | 22,571,465 |
| FKTN | 9 | 108,366,734 | CTBP2 | 10 | 126,717,714 |
| DOK6 | 18 | 67,190,086 | CDH13 | 16 | 83,177,332 |
| XPR1 | 1 | 180,853,719 | GFRA1 | 10 | 117,967,808 |
| LOC100130887 | 10 | 123,688,152 | ATXN3 | 14 | 92,573,993 |
| SRRM4 | 12 | 119,559,044 | RGS6 | 14 | 72,415,017 |
| ERC2 | 3 | 55,586,738 | FAM186A | 12 | 50,727,811 |
| PACRG | 6 | 163,721,073 | KCNIP1 | 5 | 169,816,977 |
| CNTNAP2 | 7 | 146,193,534 | PARD3B | 2 | 205,875,785 |
| CNTNAP2 | 7 | 147,654,425 | 9-Sep | 17 | 75,398,498 |
| IGF1R | 15 | 99,449,683 | ITGA1 | 5 | 52,249,612 |
| ITGA1 | 5 | 52,248,873 | ASAP1 | 8 | 131,076,710 |
| ARVCF | 22 | 19,973,205 | F13A1 | 6 | 6,152,140 |
| ANK3 | 10 | 61,822,622 | PCDH15 | 10 | 56,266,165 |
| DLG2 | 11 | 84,540,424 | CYP4V2 | 4 | 187,133,031 |
| ALS2 | 2 | 202,587,653 | ERBB4 | 2 | 213,275,113 |
| LOC100505973 | 21 | 20,933,344 | SAMD12 | 8 | 119,611,476 |
| CSMD1 | 8 | 4,492,162 | ZNF169 | 9 | 97,064,989 |
| SGCZ | 8 | 14,975,143 | XPR1 | 1 | 180,853,815 |
| SGCZ | 8 | 14,982,457 | ASPHD2 | 22 | 26,830,285 |
| DENND5B | 12 | 31,539,303 | FMNL2 | 2 | 153,371,471 |
| CNTNAP2 | 7 | 146,584,080 | CGNL1 | 15 | 57,882,297 |
| SLC25A21 | 14 | 37,261,482 | GPC6 | 13 | 94,702,014 |
| CAMK2D | 4 | 114,393,744 | APCDD1 | 18 | 10,488,091 |
| MICAL2 | 11 | 12,147,395 | PRUNE2 | 9 | 79,229,024 |
| ARVCF | 22 | 19,969,106 | SAMD4A | 14 | 55,055,645 |
| MBP | 18 | 74,782,653 | RORA | 15 | 61,127,206 |
| NALCN | 13 | 101,855,309 | MACROD2 | 20 | 15,572,198 |
| CSMD1 | 8 | 3,154,726 | ITPR1 | 3 | 4,801,017 |
| GAS7 | 17 | 9,902,914 | TRPC4 | 13 | 38,227,229 |
| PARD3B | 2 | 205,929,857 | MEPE | 4 | 88,767,942 |
| RIMS1 | 6 | 73,112,922 | RARB | 3 | 25,513,209 |
| SORCS3 | 10 | 106,591,813 | LRP1B | 2 | 142,795,322 |
| TMC5 | 16 | 19,499,993 | RBFOX2 | 22 | 36,444,188 |
| LOC100505501 | 8 | 60,032,541 | BIK | 22 | 43,498,114 |
| CDH13 | 16 | 82,831,642 | BLZF1 | 1 | 169,337,515 |
| NEDD4L | 18 | 55,783,683 | CSMD1 | 8 | 4,812,593 |
| DAOA-AS1 | 13 | 106,115,591 | PCP4L1 | 1 | 161,219,555 |
| ATP2B2 | 3 | 10,449,459 | PI4KA; | 22 | 21,141,434 |
| | | | SERPIND1 | | |
| MTIF3 | 13 | 28,024,694 | SLC35F3 | 1 | 234,119,810 |
| FMNL2 | 2 | 153,371,042 | DLGAP1 | 18 | 3,569,937 |
| DLG2 | 11 | 84,529,252 | FHIT | 3 | 60,379,450 |
| PPP1R9A | 7 | 94,722,785 | FAM173B | 5 | 10,073,549 |
| CDH13 | 16 | 83,829,129 | KCNIP1 | 5 | 170,134,498 |
| RASGEF1C | 5 | 179,528,067 | MAGI2 | 7 | 78,846,556 |
| CACNA1B | 9 | 140,866,826 | SEMA5A | 5 | 9,036,790 |
| GRM8 | 7 | 126,884,788 | GRM8 | 7 | 126,884,478 |
| DLG2 | 11 | 84,465,866 | SEC16B | 1 | 177,898,579 |
| KLHL29 | 2 | 23,802,390 | PCDH17 | 13 | 58,252,801 |
| IL15 | 4 | 142,557,279 | KCND2 | 7 | 120,039,538 |
| KCND2 | 7 | 120,019,161 | MCPH1 | 8 | 6,302,183 |
| HTR1B | 6 | 78,173,382 | DLG2 | 11 | 84,434,573 |
| CTNNA2 | 2 | 80,234,384 | ERCC6 | 10 | 50,678,369 |
| PARK2 | 6 | 161,971,076 | SLC6A5 | 11 | 20,622,975 |
| ZNF638 | 2 | 71,558,924 | ARHGAP31 | 3 | 119,096,594 |
| ANGPT1 | 8 | 108,263,134 | LRP1B | 2 | 142,261,821 |
| EML1 | 14 | 100,375,707 | CERK | 22 | 47,082,159 |
| PTPRN2 | 7 | 157,748,524 | SGCZ | 8 | 14,832,287 |
| PTPRT | 20 | 40,872,429 | SPOCK1 | 5 | 136,314,013 |
| GPM6A; | 4 | 176,655,003 | KCNH1 | 1 | 210,990,713 |
| LOC100506176 | | | | | |
| QRFPR | 4 | 122,303,714 | FSTL5 | 4 | 162,876,015 |
| NTRK2 | 9 | 87,621,188 | SDK1 | 7 | 4,189,075 |
| CNTNAP2 | 7 | 148,090,584 | ASAP1 | 8 | 131,414,632 |
| GPSM1 | 9 | 139,252,879 | CSMD1 | 8 | 2,832,139 |
| C8orf34 | 8 | 69,350,425 | CNTNAP2 | 7 | 147,704,874 |
| NRXN1 | 2 | 50,147,171 | KLHL29 | 2 | 23,929,979 |
| RNF144A | 2 | 7,181,486 | NAV3 | 12 | 78,446,057 |
| PCLO | 7 | 82,720,226 | ERBB4 | 2 | 213,040,853 |
| CNTNAP2 | 7 | 146,226,654 | KDM4C | 9 | 7,013,909 |
| ERBB4 | 2 | 213,388,276 | FHIT | 3 | 60,534,147 |
| SULT4A1 | 22 | 44,232,887 | PSD3 | 8 | 18,615,974 |
| GAN | 16 | 81,411,793 | PPP1R9A | 7 | 94,550,361 |
| QRFPR | 4 | 122,303,941 | RNF144A | 2 | 7,209,885 |
| ATP10A | 15 | 25,958,797 | ANK3 | 10 | 61,800,984 |
| SLC35F3 | 1 | 234,133,139 | SLC16A4 | 1 | 110,936,383 |
| CTNNA2 | 2 | 80,242,693 | CREB5 | 7 | 28,805,889 |
| NTSR2 | 2 | 11,810,488 | KIAA0182 | 16 | 85,689,653 |
| GPC6 | 13 | 94,028,022 | WDR90 | 16 | 712,867 |
| FAM170A | 5 | 118,964,967 | AGAP1 | 2 | 236,846,042 |
| PKP4 | 2 | 159,321,822 | JPH2 | 20 | 42,806,429 |
| LINC00114 | 21 | 40,122,021 | MACROD2 | 20 | 14,399,511 |
| CSMD1 | 8 | 3,512,965 | ARVCF | 22 | 19,998,618 |
| KAZN | 1 | 15,346,531 | EXOC2 | 6 | 654,985 |
| PDE10A | 6 | 165,927,101 | ARNT2 | 15 | 80,719,387 |
| ANK3 | 10 | 61,796,546 | IL1RAP | 3 | 190,344,900 |
| ATP2B2 | 3 | 10,455,784 | CACNG4 | 17 | 64,992,951 |
| IL1RAP | 3 | 190,348,298 | IL1RAP | 3 | 190,348,515 |
| FMN2 | 1 | 240,308,147 | ITPR1 | 3 | 4,802,573 |
| NTRK2 | 9 | 87,638,506 | NRXN3 | 14 | 79,174,840 |
| WBSCR17 | 7 | 70,601,065 | GNG2 | 14 | 52,404,912 |
| CTNNA2 | 2 | 80,236,036 | LDB2 | 4 | 16,504,184 |
| PID1 | 2 | 229,889,268 | FGF14 | 13 | 102,791,267 |
| ATP2B2 | 3 | 10,447,146 | PRDM2 | 1 | 14,028,493 |
| ZNF169 | 9 | 97,065,439 | MAGI2 | 7 | 78,536,207 |
| ERG | 21 | 40,017,446 | MAGI1 | 3 | 65,537,584 |
| SGCZ | 8 | 14,852,684 | FBXO4 | 5 | 41,934,957 |
| EHD4 | 15 | 42,192,040 | EHD4 | 15 | 42,191,909 |
| GOT2 | 16 | 58,741,215 | TMEM132B | 12 | 126,140,791 |
| CREB5 | 7 | 28,796,955 | CACNA2D1 | 7 | 81,724,466 |
| CPNE5 | 6 | 36,705,200 | KCND2 | 7 | 120,131,749 |
| PEBP4 | 8 | 22,577,263 | NTRK2 | 9 | 87,622,324 |
| LRP1B | 2 | 142,572,840 | ARNTL | 11 | 13,318,566 |
| CSMD1 | 8 | 2,808,520 | ADAMTS9-AS2 | 3 | 64,852,147 |
| IFT74 | 9 | 27,063,175 | DLGAP1 | 18 | 3,681,185 |
| MCPH1 | 8 | 6,504,316 | ARNTL | 11 | 13,297,789 |
| PRKCE | 2 | 46,412,422 | MCPH1 | 8 | 6,502,359 |
| CDH13 | 16 | 82,666,139 | SKOR2 | 18 | 44,754,651 |
| MAML3 | 4 | 141,022,317 | SKOR2 | 18 | 44,746,997 |
| GABBR2 | 9 | 101,339,947 | CACNA2D3 | 3 | 54,597,829 |
| MACROD2 | 20 | 16,034,051 | NAALADL2 | 3 | 175,473,047 |
| CSMD1 | 8 | 4,309,952 | ETV1 | 7 | 14,000,421 |
| KATNAL2 | 18 | 44,526,582 | CDH13 | 16 | 82,861,704 |
| PIP5K1B | 9 | 71,363,249 | AMPH | 7 | 38,433,726 |
| CSMD1 | 8 | 4,321,410 | SAMD12 | 8 | 119,568,066 |
| GABBR2 | 9 | 101,341,893 | SPINK1 | 5 | 147,211,393 |
| FERD3L | 7 | 19,185,757 | UNC13C | 15 | 54,694,399 |
| CLSTN2 | 3 | 139,694,900 | PLCXD2 | 3 | 111,423,413 |
| ROBO1 | 3 | 78,801,897 | SDK1 | 7 | 4,246,412 |
| MACROD2 | 20 | 15,974,988 | CACNA2D1 | 7 | 81,617,702 |
| DYNC1I1 | 7 | 95,612,737 | ITGAD | 16 | 31,420,134 |
| STK31 | 7 | 23,748,048 | SAMD12 | 8 | 119,580,949 |
| NPAS3 | 14 | 33,814,728 | PLCXD2 | 3 | 111,423,342 |
| CCDC165 | 18 | 8,797,189 | CACNB2 | 10 | 18,680,963 |
| HSPA12A | 10 | 118,431,297 | NBAS | 2 | 15,402,016 |
| COL4A3; | 2 | 228,133,001 | CLSTN2 | 3 | 139,674,735 |
| LOC654841 | | | | | |
| CDH10 | 5 | 24,603,661 | FER1L6; | 8 | 125,046,232 |
| | | | FER1L6-AS1 | | |
| DNAH17 | 17 | 76,422,473 | CADPS2 | 7 | 122,147,037 |
| FSTL5 | 4 | 162,321,420 | ROBO1 | 3 | 78,805,282 |
| CLSTN2 | 3 | 139,660,269 | RIBC2 | 22 | 45,828,594 |
| OPCML | 11 | 132,697,265 | ERBB4 | 2 | 212,783,175 |
| OPCML | 11 | 132,696,752 | TRIO | 5 | 14,508,971 |
| EPHB2 | 1 | 23,142,871 | CDH13 | 16 | 82,828,582 |
| NPY | 7 | 24,322,659 | NPY | 7 | 24,321,827 |
| NKAIN3 | 8 | 63,159,013 | NRXN3 | 14 | 80,001,533 |
| KCNK10 | 14 | 88,715,733 | PSMD14 | 2 | 162,165,008 |
| OPCML | 11 | 132,661,587 | KCNA10 | 1 | 111,060,752 |
| KLHL32 | 6 | 97,371,823 | PLCB1 | 20 | 8,865,718 |
| CCDC93 | 2 | 118,676,429 | RNF144B | 6 | 18,424,430 |
| PLCB1 | 20 | 8,865,783 | CA10 | 17 | 50,171,183 |
| SCLT1 | 4 | 129,961,179 | CSMD3 | 8 | 113,288,576 |
| PARK2 | 6 | 162,874,150 | NBAS | 2 | 15,385,364 |
| GFRA2 | 8 | 21,557,285 | FHIT | 3 | 60,066,991 |
| CDH13 | 16 | 83,091,529 | FGF14 | 13 | 102,477,248 |
| PTDSS1 | 8 | 97,384,315 | TSPAN13 | 7 | 16,792,096 |
| RBFOX3 | 17 | 77,276,546 | MTSS1 | 8 | 125,686,010 |
| PHIP | 6 | 79,675,701 | ATXN1 | 6 | 16,306,204 |
| CA10 | 17 | 50,174,076 | LYPD6 | 2 | 150,329,726 |
| NAB1 | 2 | 191,515,442 | NPAS3 | 14 | 33,551,543 |
| KCNQ1; | 11 | 2,702,513 | CTNND2 | 5 | 11,368,864 |
| KCNQ1OT1 | | | | | |
| FLJ35024 | 9 | 2,495,694 | NEBL | 10 | 21,387,376 |
| NEDD4L | 18 | 55,893,217 | BAALC; | 8 | 104,178,381 |
| | | | LOC100499183 | | |
| NKAIN3 | 8 | 63,275,763 | SLC18A2 | 10 | 119,014,948 |
| SLC18A2 | 10 | 119,014,931 | HTR1B | 6 | 78,176,538 |
| GNAS | 20 | 57,444,146 | RIBC2 | 22 | 45,821,956 |
| FSTL5 | 4 | 162,517,943 | MAGI1 | 3 | 65,757,246 |
| RAB6B | 3 | 133,575,865 | BLZF1 | 1 | 169,337,376 |
| SYNRG | 17 | 35,883,586 | NPAS3 | 14 | 33,811,253 |
| MMP27 | 11 | 102,576,382 | DLGAP2 | 8 | 1,656,318 |
| MCPH1; | 8 | 6,357,611 | ZFPM2 | 8 | 106,814,656 |
| ANGPT2 | | | | | |
| ACCN1 | 17 | 31,340,390 | CNTN4 | 3 | 2,286,504 |
| INADL | 1 | 62,257,036 | DYNC1I1 | 7 | 95,613,449 |
| KCNQ3 | 8 | 133,363,129 | PCDH10 | 4 | 132,506,771 |
| PCDH10 | 4 | 132,505,315 | KCNB2 | 8 | 73,454,767 |
| PCLO | 7 | 82,764,425 | MYT1L | 2 | 1,794,410 |
| GNG2 | 14 | 52,385,962 | DOK5 | 20 | 53,267,576 |
| NECAB1 | 8 | 91,969,669 | SHROOM3 | 4 | 77,484,202 |
| CA10 | 17 | 50,067,262 | NBEA | 13 | 36,080,730 |
| PACRG | 6 | 163,613,335 | SMARCA2 | 9 | 2,082,671 |
| LINC00299 | 2 | 8,446,735 | NBEA | 13 | 36,147,469 |
| AGL | 1 | 100,327,026 | SLC7A14 | 3 | 170,179,582 |
| NPAS3 | 14 | 34,044,752 | GABBR2 | 9 | 101,233,069 |
| PPARGC1A | 4 | 23,893,017 | RASGRP1 | 15 | 38,786,114 |
| KCNMA1 | 10 | 78,934,838 | COL4A3; | 2 | 228,130,088 |
| | | | LOC654841 | | |
| KCNQ1 | 11 | 2,537,751 | CAST | 5 | 96,078,337 |
| CAST | 5 | 96,077,968 | WWOX | 16 | 78,905,235 |
| PCSK6 | 15 | 101,847,800 | MACROD2 | 20 | 15,339,430 |
| FERMT1 | 20 | 6,104,673 | FLJ35024 | 9 | 2,564,179 |
| SYNE1 | 6 | 152,487,926 | IKZF2 | 2 | 213,870,678 |
| SDK1 | 7 | 3,529,504 | FAM186A | 12 | 50,724,444 |
| NRXN3 | 14 | 79,631,747 | ATF6 | 1 | 161,761,312 |
| LOC100505806 | 5 | 9,546,995 | LOC100505806 | 5 | 9,547,941 |
| MAP1B | 5 | 71,445,991 | CNOT2 | 12 | 70,645,867 |
| PDE4D | 5 | 58,782,554 | CSMD1 | 8 | 3,015,710 |
| LOC100129434 | 2 | 56,410,666 | CA10 | 17 | 50,155,850 |
| KCNB1 | 20 | 47,988,601 | SGCZ | 8 | 14,149,802 |
| ELMOD1 | 11 | 107,474,795 | NEDD9 | 6 | 11,234,164 |
| CDH13 | 16 | 82,681,884 | CCDC165 | 18 | 8,814,140 |
| NKAIN3 | 8 | 63,903,178 | RORA | 15 | 61,130,792 |
| CLSTN2 | 3 | 140,266,593 | CA10 | 17 | 50,172,676 |
| ACCS | 11 | 44,085,369 | ATRN | 20 | 3,628,312 |
| KCNIP4 | 4 | 21,333,470 | PTPRN2 | 7 | 158,151,977 |
| NRXN3 | 14 | 79,983,905 | ST8SIA1 | 12 | 22,347,663 |
| FMN2 | 1 | 240,463,718 | DGKB | 7 | 14,234,314 |
| KCNB2 | 8 | 73,519,816 | CERS5 | 12 | 50,526,814 |
| SKOR2 | 18 | 44,751,856 | NRXN3 | 14 | 80,078,238 |
| MAGI2 | 7 | 78,316,880 | ATP2B2 | 3 | 10,454,880 |
| RBFOX3 | 17 | 77,270,593 | AKAP9 | 7 | 91,712,698 |
| GNG2 | 14 | 52,389,257 | GABRR2 | 6 | 89,996,405 |
| PACRG | 6 | 163,623,169 | MACROD2 | 20 | 15,323,521 |
| FBLN7 | 2 | 112,940,578 | FBLN7 | 2 | 112,939,548 |
| KCNMA1 | 10 | 78,951,780 | PDE1C | 7 | 31,881,291 |
| EMID2 | 7 | 101,202,565 | CSMD1 | 8 | 4,305,023 |
| CSMD1 | 8 | 3,788,431 | EPHB2 | 1 | 23,141,653 |
| DLG2 | 11 | 83,479,572 | LOC100505985 | 6 | 50,294,443 |
| RYR2 | 1 | 237,427,945 | DGKI | 7 | 137,130,197 |
| CD247 | 1 | 167,400,074 | KCNQ1 | 11 | 2,639,712 |
| SPINK1 | 5 | 147,234,756 | GRB10 | 7 | 50,673,363 |
| SVEP1 | 9 | 113,199,768 | SLC22A16 | 6 | 110,745,977 |
| EPAS1 | 2 | 46,523,934 | COMMD1 | 2 | 62,153,717 |
| NRCAM | 7 | 108,074,766 | MGAT2 | 14 | 50,089,696 |
| TSPAN9 | 12 | 3,256,711 | MAGI2 | 7 | 78,265,298 |
| PARK2 | 6 | 162,877,216 | PRKG1 | 10 | 53,425,550 |
| NKAIN3 | 8 | 63,883,904 | TRPM3 | 9 | 73,163,235 |
| NCS1 | 9 | 132,998,557 | PIK3C2G | 12 | 18,634,528 |
| TSPAN9 | 12 | 3,251,901 | ROBO1 | 3 | 79,013,878 |
| VCAN | 5 | 82,848,642 | PREX1 | 20 | 47,320,829 |
| ARHGAP21 | 10 | 24,929,560 | NRG3 | 10 | 83,090,548 |
| LOC100506689 | 8 | 102,503,717 | ATP2B2 | 3 | 10,454,406 |
| ZNF536 | 19 | 31,137,357 | ZNF536 | 19 | 31,118,591 |
| FHIT | 3 | 60,217,330 | RGS7 | 1 | 241,070,371 |
| CA10 | 17 | 50,067,961 | NALCN | 13 | 102,024,360 |
| NAV2 | 11 | 19,885,338 | RYR2 | 1 | 237,416,049 |
| EXOC2 | 6 | 514,721 | FERD3L | 7 | 19,184,059 |
| NBEA | 13 | 36,185,434 | GPC6 | 13 | 93,999,944 |
| CCDC50 | 3 | 191,112,226 | GABBR2 | 9 | 101,339,629 |
| PTPRT | 20 | 41,421,036 | ITPR1 | 3 | 4,781,815 |
| ATRN | 20 | 3,629,254 | ATRN | 20 | 3,631,881 |
| MTUS2 | 13 | 30,079,826 | ELOVL7 | 5 | 60,049,119 |
| CHMP6 | 17 | 78,973,474 | NBAS | 2 | 15,310,606 |
| GABRR2 | 6 | 90,021,541 | DCAF11 | 14 | 24,583,846 |
| PON1 | 7 | 94,955,221 | MACROD2 | 20 | 15,312,488 |
| C19orf45 | 19 | 7,573,098 | CDH13 | 16 | 83,814,659 |
| KCNB2 | 8 | 73,455,936 | SLC1A1 | 9 | 4,516,768 |
| FAM104A | 17 | 71,205,036 | LEPREL1 | 3 | 189,842,190 |
| NBEA | 13 | 36,119,563 | RYR2 | 1 | 237,487,678 |
| ASTN2 | 9 | 119,262,849 | MACROD2 | 20 | 16,034,339 |
| KCNQ3 | 8 | 133,413,386 | PTPN5 | 11 | 18,815,270 |
| ITPR1 | 3 | 4,622,318 | ITPR1 | 3 | 4,619,381 |
| DPP10 | 2 | 116,503,671 | CELF2 | 10 | 11,139,698 |
| MUC7 | 4 | 71,346,701 | TRPC4 | 13 | 38,216,068 |
| CCDC165 | 18 | 8,787,857 | IGSF22 | 11 | 18,747,959 |
| SGCZ | 8 | 14,675,294 | LINC00308 | 21 | 24,062,292 |
| NCAM2 | 21 | 22,910,051 | CA10 | 17 | 50,067,848 |
| CSMD1 | 8 | 3,518,993 | SGCZ | 8 | 14,746,634 |
| SLC17A8 | 12 | 100,813,976 | ACYP2 | 2 | 54,509,549 |
| GRHL2 | 8 | 102,586,388 | CNTNAP2 | 7 | 148,117,730 |
| SHC3 | 9 | 91,675,560 | ESRRG | 1 | 216,676,445 |
| PDE1C | 7 | 32,111,306 | CTNNA2 | 2 | 80,513,925 |
| GRID2 | 4 | 94,295,669 | DLC1 | 8 | 13,057,273 |
| PTGER3 | 1 | 71,477,539 | SLC4A1AP | 2 | 27,887,034 |
| ERC2 | 3 | 56,012,933 | MYO5B | 18 | 47,352,134 |
| STXBP5L | 3 | 120,658,659 | PTPRG | 3 | 61,728,839 |
| PCDH7 | 4 | 30,863,804 | MAGI2 | 7 | 78,246,671 |
| DNAH9 | 17 | 11,814,378 | DNAH9 | 17 | 11,811,666 |
| ATRNL1 | 10 | 117,445,235 | RYR3 | 15 | 33,732,098 |
| ROBO2 | 3 | 77,565,844 | ITPR1 | 3 | 4,625,437 |
| NRXN3 | 14 | 79,633,990 | FHIT | 3 | 60,216,250 |
| CHMP6 | 17 | 78,973,901 | PACRG | 6 | 163,618,100 |
| TBC1D2B | 15 | 78,303,748 | SGCZ | 8 | 14,737,262 |
| WWOX | 16 | 78,933,487 | ELFN2 | 22 | 37,766,196 |
| PCSK6 | 15 | 101,854,583 | PLCG2 | 16 | 81,849,382 |
| OPCML | 11 | 132,651,586 | NPAS3 | 14 | 33,948,400 |
| PIKFYVE | 2 | 209,206,633 | PARK2 | 6 | 162,144,016 |
| KCNMA1 | 10 | 79,183,038 | SORBS1 | 10 | 97,271,827 |
| NLGN1 | 3 | 173,335,268 | NPAS3 | 14 | 33,662,267 |
| NRG3 | 10 | 83,933,250 | CNTN4 | 3 | 2,913,017 |
| GREM2 | 1 | 240,652,947 | NPAS3 | 14 | 33,668,714 |
| SORBS1 | 10 | 97,295,395 | FHIT | 3 | 60,082,619 |
| ARSB | 5 | 78,135,241 | COL22A1 | 8 | 139,622,943 |
| COL22A1 | 8 | 139,616,386 | RGS7 | 1 | 241,258,422 |
| ETV1 | 7 | 14,030,119 | ROBO2 | 3 | 77,382,285 |
| NAV2 | 11 | 19,868,759 | GRID2 | 4 | 94,250,785 |
| ETV1 | 7 | 13,976,258 | COL4A4 | 2 | 227,867,385 |
| NALCN | 13 | 101,820,926 | CNTNAP2 | 7 | 146,588,537 |
| NAV2 | 11 | 19,902,266 | NPFFR2 | 4 | 73,003,569 |
| ETV1 | 7 | 14,029,772 | ETV1 | 7 | 14,029,739 |
| ETV1 | 7 | 14,030,093 | LRP1B | 2 | 141,644,560 |
| MYO3B | 2 | 171,260,797 | CSMD1 | 8 | 3,031,069 |
| TMEFF2 | 2 | 193,057,128 | ARMC3 | 10 | 23,214,761 |
| L3MBTL4 | 18 | 5,954,871 | PLCG2 | 16 | 81,977,741 |
| PSD3 | 8 | 18,385,538 | PPP2R2B | 5 | 146,460,691 |
| LOC100289230 | 5 | 98,548,315 | DCC | 18 | 49,896,109 |
| DOK6 | 18 | 67,279,176 | CSMD1 | 8 | 3,351,147 |
| TMEFF2 | 2 | 193,055,583 | DGKB | 7 | 14,665,239 |
| DOK6 | 18 | 67,281,637 | SLC6A1 | 3 | 11,055,705 |
| ADAMTS9 | 3 | 64,546,459 | NLGN1 | 3 | 173,310,633 |
| FHIT | 3 | 60,070,931 | EPHA6 | 3 | 96,849,058 |
| FHIT | 3 | 60,438,854 | LRP1B | 2 | 141,639,645 |
| PTPRT | 20 | 41,179,698 | DCTN4 | 5 | 150,097,883 |
| MACROD2 | 20 | 14,747,025 | ANK3 | 10 | 61,957,641 |
| RGS7 | 1 | 241,242,460 | SVEP1 | 9 | 113,150,431 |
| KAZN | 1 | 15,293,051 | PTPRG | 3 | 62,160,653 |
| GPM6A | 4 | 176,566,472 | RORA | 15 | 61,525,619 |
| TRAPPC10 | 21 | 45,479,712 | NRP2 | 2 | 206,545,421 |
| DNAH5 | 5 | 13,755,378 | BNIP2 | 15 | 59,955,668 |
| NRG3 | 10 | 84,230,405 | 9-Sep | 17 | 75,496,361 |
| NLGN1 | 3 | 173,902,010 | BTN3A1 | 6 | 26,420,425 |
| BTN3A1 | 6 | 26,414,483 | BTN3A1 | 6 | 26,415,798 |
| KYNU | 2 | 143,717,774 | MSR1 | 8 | 15,967,438 |
| MSR1 | 8 | 15,967,078 | PLA2G2D | 1 | 20,439,522 |
| CDH7 | 18 | 63,530,016 | NLGN1 | 3 | 173,936,752 |
| TLN2 | 15 | 63,133,002 | ZNF532 | 18 | 56,587,802 |
| PRICKLE2 | 3 | 64,191,981 | 9-Sep | 17 | 75,496,342 |
| CCDC93 | 2 | 118,677,813 | CSMD1 | 8 | 4,372,185 |
| CNTN4 | 3 | 2,741,787 | NCS1 | 9 | 132,963,115 |
| LOC286094 | 8 | 136,070,005 | CACNA2D1 | 7 | 81,669,017 |
| KCNIP4 | 4 | 20,870,617 | MAGI2 | 7 | 78,748,068 |
| PCSK6 | 15 | 101,861,383 | MYO10 | 5 | 16,706,766 |
| OPCML | 11 | 133,167,123 | CLSTN2 | 3 | 139,710,833 |
| MACROD2 | 20 | 15,684,293 | GALNTL4 | 11 | 11,420,192 |
| DGKB | 7 | 14,204,193 | DLG2 | 11 | 84,546,793 |
| CRISPLD1 | 8 | 75,906,641 | FHIT | 3 | 59,871,049 |
| TFB1M | 6 | 155,637,025 | EPHB1 | 3 | 134,655,266 |
| PDE4D | 5 | 58,333,097 | NRG3 | 10 | 84,225,687 |
| MSI2 | 17 | 55,749,233 | RYR3 | 15 | 34,099,483 |
| THADA | 2 | 43,783,241 | SLCO3A1 | 15 | 92,531,229 |
| GPR97 | 16 | 57,722,833 | DUOX2 | 15 | 45,383,446 |
| ATXN1 | 6 | 16,514,083 | PARK2 | 6 | 162,499,315 |
| SMEK2 | 2 | 55,776,793 | CNTNAP2 | 7 | 148,037,751 |
| ERG | 21 | 39,824,245 | NELL1 | 11 | 20,698,929 |
| CACNA2D1 | 7 | 81,942,310 | SMARCA2 | 9 | 2,177,170 |
| PIK3CG | 7 | 106,546,087 | GRM5 | 11 | 88,241,196 |
| PRKCE | 2 | 46,335,714 | ZFPM2 | 8 | 106,589,247 |
| PJA2 | 5 | 108,672,755 | EMID2 | 7 | 101,005,926 |
| KCNQ1 | 11 | 2,464,728 | SFRP1 | 8 | 41,155,258 |
| PARD3B | 2 | 205,416,821 | NALCN | 13 | 101,815,002 |
| NALCN | 13 | 101,816,161 | CSMD1 | 8 | 3,648,320 |
| MAGI2 | 7 | 78,107,199 | ARHGAP15 | 2 | 144,383,727 |
| CDH7 | 18 | 63,475,059 | CDH7 | 18 | 63,470,003 |
| DOK6 | 18 | 67,284,044 | LRP1B | 2 | 141,641,929 |
| GALNT9 | 12 | 132,689,052 | TRIM9 | 14 | 51,495,398 |
| SOBP | 6 | 107,980,406 | HAAO | 2 | 42,995,840 |
| CDH13 | 16 | 83,628,618 | PTPRT | 20 | 40,728,095 |
| CACNA2D3 | 3 | 54,467,289 | INMT- | 7 | 30,817,249 |
| | | | FAM188B; | | |
| | | | FAM188B | | |
| CTNNA2 | 2 | 79,760,772 | PLCG2 | 16 | 81,916,163 |
| MACROD2 | 20 | 14,734,270 | C13orf35 | 13 | 113,288,426 |
| RIMBP2 | 12 | 130,888,767 | MSR1 | 8 | 15,965,906 |
| CREB3L2 | 7 | 137,671,487 | RNF144B | 6 | 18,389,833 |
| UNC13C | 15 | 54,805,350 | FMNL2 | 2 | 153,405,594 |
| LOC100128590; | 2 | 40,455,984 | SDK2 | 17 | 71,362,083 |
| SLC8A1 | | | | | |
| WWOX | 16 | 78,590,925 | CPLX2 | 5 | 175,223,616 |
| MYO10 | 5 | 16,666,180 | CNTN6 | 3 | 1,319,496 |
| CTNNA2 | 2 | 80,657,752 | OPCML | 11 | 133,148,455 |
| KCNK2 | 1 | 215,298,570 | NAV3 | 12 | 78,319,532 |
| NLGN1 | 3 | 173,921,634 | KCNB1 | 20 | 47,989,624 |
| TMC1 | 9 | 75,135,825 | MCPH1 | 8 | 6,422,141 |
| MACROD2 | 20 | 14,768,903 | SHC3 | 9 | 91,637,142 |
| MAGI2 | 7 | 78,692,139 | PSD3 | 8 | 18,786,787 |
| GALNTL4 | 11 | 11,319,245 | MAGI2 | 7 | 78,764,223 |
| NBEA | 13 | 35,140,485 | CDH13 | 16 | 83,023,920 |
| ABCA4 | 1 | 94,476,467 | OPCML | 11 | 132,544,060 |
| CADPS | 3 | 62,788,035 | RYR2 | 1 | 237,858,185 |
| MUSK | 9 | 113,579,789 | NCEH1 | 3 | 172,350,135 |
| KCNQ3 | 8 | 133,418,396 | NLGN1 | 3 | 173,357,992 |
| INMT- | 7 | 30,842,257 | DENND4C | 9 | 19,343,291 |
| FAM188B; | | | | | |
| FAM188B | | | | | |
| LRP1B | 2 | 141,242,918 | KCNJ3 | 2 | 155,586,570 |
| PRKG1 | 10 | 53,853,246 | DGKB | 7 | 14,305,589 |
| ATXN1 | 6 | 16,497,883 | SNTG1 | 8 | 50,822,736 |
| CERK | 22 | 47,093,846 | C15orf41 | 15 | 36,931,320 |
| MUSK | 9 | 113,430,771 | NRG3 | 10 | 84,231,385 |
| EPHA7 | 6 | 93,950,764 | CTNNA2 | 2 | 80,307,777 |
| NAV2 | 11 | 19,770,927 | GRID2 | 4 | 94,250,702 |
| LRFN2 | 6 | 40,458,625 | ATP2B2 | 3 | 10,448,426 |
| NALCN | 13 | 101,900,696 | DOK6 | 18 | 67,286,267 |
| OPCML | 11 | 132,554,005 | MYO3A | 10 | 26,250,566 |
| KCNIP4 | 4 | 20,856,597 | STX11 | 6 | 144,508,698 |
| STX11 | 6 | 144,512,989 | IRF8 | 16 | 86,010,523 |
| IRF8 | 16 | 86,009,519 | SORBS1 | 10 | 97,272,652 |
| RGS7 | 1 | 241,125,990 | ST8SIA1 | 12 | 22,348,998 |
| NPAS3 | 14 | 33,576,437 | DLC1 | 8 | 13,000,629 |
| NFIL3 | 9 | 94,187,265 | SLC35F3 | 1 | 234,039,968 |
| CSMD1 | 8 | 3,645,978 | FOXP1 | 3 | 71,156,340 |
| CCBE1 | 18 | 57,365,526 | LOC100128590; | 2 | 40,455,568 |
| | | | SLC8A1 | | |
| PLCG2 | 16 | 81,982,491 | TMTC2 | 12 | 83,526,911 |
| CHRM3 | 1 | 239,821,058 | CNTN4 | 3 | 2,582,255 |
| EPHB1 | 3 | 134,554,163 | LOC100128590; | 2 | 40,455,632 |
| | | | SLC8A1 | | |
| MAGI2 | 7 | 78,701,601 | RGS7 | 1 | 241,442,019 |
| JAG1 | 20 | 10,621,305 | EXOC2 | 6 | 593,109 |
| EMID2 | 7 | 101,159,950 | SEMA5A | 5 | 9,380,118 |
| CLSTN2 | 3 | 139,700,967 | SAMD4A | 14 | 55,124,327 |
| NLGN1 | 3 | 173,932,956 | MUSK | 9 | 113,580,133 |
| NCAM2 | 21 | 22,511,763 | NEDD4L | 18 | 55,731,712 |
| FOXP1 | 3 | 71,430,995 | ITPR2 | 12 | 26,986,198 |
| ERG | 21 | 39,909,266 | GPR116 | 6 | 46,827,239 |
| FMN2 | 1 | 240,358,173 | TMEM132E | 17 | 32,966,065 |
| TMEM132E | 17 | 32,966,119 | KCNMA1 | 10 | 79,294,680 |
| CADPS | 3 | 62,761,047 | CA10 | 17 | 49,811,358 |
| FLJ38109 | 5 | 153,815,054 | KCNQ1 | 11 | 2,755,346 |
| SELT | 3 | 150,346,654 | USP10 | 16 | 84,790,559 |
| NRCAM | 7 | 107,939,793 | LRRK1 | 15 | 101,606,889 |
| SGCZ | 8 | 13,955,788 | LDB2 | 4 | 16,508,781 |
| PACRG | 6 | 163,303,962 | TGIF1 | 18 | 3,432,439 |
| MIR3974 | 12 | 17,358,689 | FREM1 | 9 | 14,816,829 |
| MDGA2 | 14 | 47,812,311 | ADAMTS9; | 3 | 64,672,474 |
| | | | ADAMTS9-AS2 | | |
| NRCAM | 7 | 107,938,376 | FBXL2 | 3 | 33,428,373 |
| PARD3B | 2 | 205,775,783 | CTNND2 | 5 | 11,161,237 |
| PTPRT | 20 | 41,138,259 | SEC23B | 20 | 18,488,065 |
| PKP4 | 2 | 159,320,951 | TSC1 | 9 | 135,767,943 |
| PDE4D | 5 | 58,293,664 | GRID2 | 4 | 93,453,356 |
| IYD | 6 | 150,719,380 | CNTN6 | 3 | 1,296,226 |
| DOK6 | 18 | 67,282,973 | MYO10 | 5 | 16,883,395 |
| CLSTN2 | 3 | 139,931,872 | RAB6B | 3 | 133,615,546 |
| RAB6B | 3 | 133,616,135 | CTNND2 | 5 | 11,291,983 |
| RAB6B | 3 | 133,615,263 | RYR3 | 15 | 33,621,235 |
| RPRD1A | 18 | 33,571,268 | SORBS1 | 10 | 97,083,979 |
| GNG2 | 14 | 52,360,813 | ARNTL | 11 | 13,325,695 |
| NCAM2 | 21 | 22,864,581 | KIAA0182 | 16 | 85,683,171 |
| SH2D4B | 10 | 82,865,385 | EXOC2 | 6 | 633,557 |
| AKAP9 | 7 | 91,708,898 | DOK6 | 18 | 67,264,134 |
| ROBO2 | 3 | 77,604,690 | INPP4A | 2 | 99,133,611 |
| CDH13 | 16 | 83,106,301 | RGS7 | 1 | 241,115,683 |
| DLG2 | 11 | 84,456,440 | PLXDC2 | 10 | 20,105,936 |
| PIKFYVE | 2 | 209,214,407 | CSMD1 | 8 | 4,493,225 |
| CNTNAP2 | 7 | 147,935,699 | OSBPL1A | 18 | 21,828,835 |
| NPY | 7 | 24,324,759 | CSMD1 | 8 | 3,807,943 |
| CCDC85A | 2 | 56,460,013 | RYR2 | 1 | 237,383,271 |
| CCDC85A | 2 | 56,596,394 | NAV3 | 12 | 78,301,608 |
| HYDIN | 16 | 70,926,334 | CSMD1 | 8 | 4,493,493 |
| NPY | 7 | 24,320,646 | MACROD2 | 20 | 15,503,398 |
| RAB6B | 3 | 133,616,371 | FMN2 | 1 | 240,564,616 |
| PSD3 | 8 | 18,792,419 | MSI2 | 17 | 55,748,408 |
| WBSCR17 | 7 | 70,738,028 | CTNND2 | 5 | 11,166,899 |
| TOX | 8 | 60,018,743 | NRG3 | 10 | 84,315,759 |
| DPP6 | 7 | 153,896,243 | SORBS1 | 10 | 97,123,793 |
| SNCA | 4 | 90,682,504 | CACNG2 | 22 | 37,024,953 |
| PLCB1 | 20 | 8,490,081 | HAAO | 2 | 42,997,614 |
| KCNB2 | 8 | 73,736,766 | ARHGAP21 | 10 | 24,929,141 |
| PREX2 | 8 | 68,910,473 | LRP1B | 2 | 142,488,592 |
| AGAP1 | 2 | 236,995,045 | NPAS3 | 14 | 33,567,750 |
| TMEFF2 | 2 | 192,922,256 | C7orf58 | 7 | 120,876,835 |
| CAMK2D | 4 | 114,384,328 | CACNB4 | 2 | 152,695,191 |
| SLC35F3 | 1 | 234,121,873 | DNAH17 | 17 | 76,491,309 |
| EXOC2 | 6 | 538,063 | FHIT | 3 | 60,271,669 |
| DENND4C | 9 | 19,290,143 | NRCAM | 7 | 107,907,393 |
| LRP1B | 2 | 142,391,367 | MAMDC2; | 9 | 72,834,056 |
| | | | LOC100507299 | | |
| POLR2M | 15 | 57,999,304 | CTBP2 | 10 | 126,683,857 |
| KCNB2 | 8 | 73,484,874 | MAML3 | 4 | 141,035,921 |
| EPHA4 | 2 | 222,283,000 | NRXN3 | 14 | 79,651,600 |
| DLEU2 | 13 | 50,855,108 | LRRK1 | 15 | 101,609,737 |
| NRG3 | 10 | 84,606,682 | ROBO1 | 3 | 78,685,024 |
| CCDC50 | 3 | 191,087,740 | TBC1D22A | 22 | 47,310,557 |
| LRP1B | 2 | 142,876,051 | CBLB | 3 | 105,399,476 |
| PLCG2 | 16 | 81,912,081 | CHRM3 | 1 | 239,844,600 |
| APBB2 | 4 | 40,812,669 | DHODH | 16 | 72,058,881 |
| ALK | 2 | 29,543,736 | RORA | 15 | 61,151,622 |
| GBE1 | 3 | 81,812,406 | GLDN | 15 | 51,687,839 |
| SNX21; ACOT8 | 20 | 44,471,340 | MSI2 | 17 | 55,710,880 |
| NALCN | 13 | 101,747,265 | BNC2 | 9 | 16,416,995 |
| CHRM3 | 1 | 239,824,248 | NRXN3 | 14 | 78,920,327 |
| PTPRN2 | 7 | 157,642,767 | CSMD1 | 8 | 4,641,941 |
| CHN2 | 7 | 29,554,284 | NBN | 8 | 90,946,601 |
| EXOC2 | 6 | 534,527 | GAS7 | 17 | 9,999,817 |
| SAG | 2 | 234,229,320 | SORCS2 | 4 | 7,743,283 |
| TMX2-CTNND1 | 11 | 57,525,883 | GRB10 | 7 | 50,801,117 |
| GRB10 | 7 | 50,801,917 | DTNBP1 | 6 | 15,628,102 |
| DTNBP1 | 6 | 15,651,132 | IQGAP2 | 5 | 75,964,507 |
| DTNBP1 | 6 | 15,653,649 | SERPINI1 | 3 | 167,455,005 |
| GLP1R | 6 | 39,054,589 | CDH13 | 16 | 82,779,603 |
| C14orf182 | 14 | 50,473,098 | ATRNL1 | 10 | 116,875,810 |
| ATXN3 | 14 | 92,525,145 | PCSK5 | 9 | 78,778,550 |
| NPAS3 | 14 | 34,116,992 | PJA2 | 5 | 108,747,204 |
| ARPP21 | 3 | 35,712,071 | NCAM2 | 21 | 22,503,372 |
| NPAS3 | 14 | 34,127,481 | KIAA0947 | 5 | 5,818,164 |
| CGNL1 | 15 | 57,712,536 | NXPH2 | 2 | 139,428,096 |
| CGNL1 | 15 | 57,817,191 | NPAS3 | 14 | 33,806,438 |
| ODZ3 | 4 | 183,240,892 | PLA2G1B | 12 | 120,760,911 |
| SKAP1 | 17 | 46,262,171 | WWOX | 16 | 78,324,042 |
| ITPR1 | 3 | 4,870,000 | DGKB | 7 | 14,596,054 |
| ATF3 | 1 | 212,793,849 | VPS41 | 7 | 38,949,424 |
| AKAP13 | 15 | 86,291,013 | ABI2 | 2 | 204,192,201 |
| ROBO1 | 3 | 78,977,507 | PTPRG | 3 | 62,038,177 |
| FLJ22447 | 14 | 62,120,575 | RYR2 | 1 | 237,201,051 |
| PKIA | 8 | 78,951,278 | ARPP21 | 3 | 35,715,712 |
| PDE1C | 7 | 31,961,538 | CBLB | 3 | 105,425,910 |
| MYL12B | 18 | 3,327,868 | CSMD1 | 8 | 4,626,927 |
| LOXL2 | 8 | 23,155,337 | KAZN | 1 | 15,275,515 |
| LRP1B | 2 | 142,179,858 | VTI1A | 10 | 114,575,767 |
| SULT4A1 | 22 | 44,229,793 | SULT4A1 | 22 | 44,236,864 |
| KCNB2 | 8 | 73,777,863 | UNC5C | 4 | 96,390,341 |
| ARPP21 | 3 | 35,728,881 | LOC100506731 | 14 | 85,995,799 |
| CBLB | 3 | 105,432,459 | PARD3B | 2 | 206,215,828 |
| TBC1D1 | 4 | 37,904,456 | EVC | 4 | 5,804,830 |
| MR1 | 1 | 181,025,110 | EPHB2 | 1 | 23,171,706 |
| BAALC | 8 | 104,229,824 | ABT1 | 6 | 26,600,156 |
| CLASP2 | 3 | 33,537,513 | GLDN | 15 | 51,648,597 |
| TMEFF2 | 2 | 192,959,316 | BAALC | 8 | 104,238,747 |
| GDA | 9 | 74,863,887 | CCBE1 | 18 | 57,365,344 |
| NPAS3 | 14 | 34,115,818 | ARFGAP3 | 22 | 43,192,480 |
| PLA2G4D | 15 | 42,391,075 | TMX2- | 11 | 57,550,785 |
| | | | CTNND1; | | |
| | | | CTNND1 | | |
| TMEM163 | 2 | 135,288,375 | NPAS3 | 14 | 33,439,678 |
| RAB11FIP4 | 17 | 29,717,879 | COL22A1 | 8 | 139,758,550 |
| NALCN | 13 | 101,749,365 | NALCN | 13 | 101,744,417 |
| PLEKHH2 | 2 | 43,884,582 | CNTNAP2 | 7 | 146,541,290 |
| SAMD12 | 8 | 119,206,468 | SAMD12 | 8 | 119,207,140 |
| SAMD12 | 8 | 119,207,381 | RORA | 15 | 61,164,545 |
| ZNF365 | 10 | 64,307,317 | MICAL2 | 11 | 12,169,917 |
| PRKCE | 2 | 46,344,717 | CARD11 | 7 | 3,012,242 |
| ST8SIA2 | 15 | 93,012,351 | GBE1 | 3 | 81,539,382 |
| QRFP | 9 | 133,769,786 | PARD3B | 2 | 205,898,117 |
| CGNL1 | 15 | 57,843,391 | IP6K1 | 3 | 49,762,662 |
| TBC1D22A | 22 | 47,569,605 | GRID2 | 4 | 93,452,091 |
| PAPPA | 9 | 119,024,929 | CGNL1 | 15 | 57,674,534 |
| LOC286190; | 8 | 71,549,614 | PSD3 | 8 | 18,636,688 |
| LACTB2 | | | | | |
| SLC2A9 | 4 | 9,828,745 | NCAM2 | 21 | 22,503,145 |
| CHN2 | 7 | 29,500,433 | SAAL1 | 11 | 18,091,665 |
| SLC41A1 | 1 | 205,759,195 | DOK6 | 18 | 67,400,139 |
| LOC728755 | 14 | 27,620,796 | CSMD1 | 8 | 3,214,267 |
| PDE1C | 7 | 31,962,443 | ANO2 | 12 | 5,698,059 |
| DEAF1 | 11 | 674,259 | TRIP12 | 2 | 230,657,496 |
| PTPRT | 20 | 40,821,926 | MTSS1 | 8 | 125,587,871 |
| HS1BP3 | 2 | 20,818,883 | PRKCE | 2 | 46,343,735 |
| NTRK2 | 9 | 87,704,881 | CLSTN2 | 3 | 139,714,321 |
| FAM69A | 1 | 93,308,853 | BIRC6 | 2 | 32,822,957 |
| EXOC4 | 7 | 133,424,668 | EXOC2 | 6 | 531,483 |
| ADAMTS19 | 5 | 129,074,369 | ADAMTS19 | 5 | 129,074,560 |
| ADAMTS19 | 5 | 129,072,943 | CCDC165 | 18 | 8,817,778 |
| SLC2A13 | 12 | 40,150,610 | DLC1 | 8 | 12,943,065 |
| DEAF1 | 11 | 644,325 | PPM1H | 12 | 63,038,698 |
| LRP1B | 2 | 141,883,206 | FSTL5 | 4 | 162,823,690 |
| SYNE1 | 6 | 152,521,714 | ANO2 | 12 | 5,860,329 |
| KCNN3 | 1 | 154,838,050 | ERBB4 | 2 | 212,240,337 |
| NCAM2 | 21 | 22,502,758 | CAMKV | 3 | 49,896,618 |
| ARPP21 | 3 | 35,718,847 | CDH13 | 16 | 83,426,932 |
| CSMD1 | 8 | 3,135,844 | FLJ45139 | 21 | 40,250,008 |
| NPAS3 | 14 | 34,136,476 | SORBS1 | 10 | 97,081,500 |
| C15orf41 | 15 | 36,948,788 | SLC2A13 | 12 | 40,151,546 |
| SLC2A13 | 12 | 40,151,891 | ARL13B; | 3 | 93,738,694 |
| | | | STX19 | | |
| HHAT | 1 | 210,570,783 | FAM69A | 1 | 93,307,908 |
| QPCT | 2 | 37,600,427 | SYNE1 | 6 | 152,453,291 |
| PLD5 | 1 | 242,545,880 | NAV2 | 11 | 19,787,053 |
| LPHN3 | 4 | 62,919,707 | RORA | 15 | 61,155,328 |
| PRKG1 | 10 | 53,615,000 | DFNB31 | 9 | 117,169,935 |
| DFNB31 | 9 | 117,167,192 | PDE1C | 7 | 32,021,399 |
| RORA | 15 | 61,154,619 | TOX | 8 | 59,946,434 |
| GLDN | 15 | 51,648,847 | ST8SIA2 | 15 | 93,007,974 |
| FSTL5 | 4 | 162,807,615 | SNCA | 4 | 90,758,945 |
| DGKB | 7 | 14,284,617 | MACROD2 | 20 | 15,975,305 |
| NRG3 | 10 | 84,724,221 | CAMKMT | 2 | 44,999,709 |
| GALNTL4 | 11 | 11,312,467 | KDM4C | 9 | 7,322,335 |
| IL17RD | 3 | 57,203,398 | CDH8 | 16 | 62,002,956 |
| ATP10A | 15 | 25,925,094 | CSMD1 | 8 | 4,657,295 |
| NRG3 | 10 | 84,565,851 | DAB2IP | 9 | 124,433,681 |
| SLIT2 | 4 | 20,251,518 | LRP1B | 2 | 141,889,752 |
| PLCB1 | 20 | 8,432,314 | RYR2 | 1 | 237,944,814 |
| PCLO | 7 | 82,462,834 | FBXL17 | 5 | 107,240,149 |
| UNC5C | 4 | 96,393,071 | DOK6 | 18 | 67,393,698 |
| PTCHD4 | 6 | 47,868,517 | NPAS3 | 14 | 33,920,194 |
| HTR5A | 7 | 154,876,342 | OTOG | 11 | 17,580,175 |
| CELF2 | 10 | 11,075,552 | EPAS1 | 2 | 46,565,091 |
| ABCA1 | 9 | 107,665,978 | FRMD1 | 6 | 168,462,765 |
| SVEP1 | 9 | 113,127,180 | PCSK5 | 9 | 78,776,057 |
| DGKB | 7 | 14,282,726 | PTPRM | 18 | 7,564,653 |
| KCNIP1 | 5 | 170,038,936 | CACNA2D3 | 3 | 54,257,411 |
| CDH23 | 10 | 73,392,466 | NCAM2 | 21 | 22,505,742 |
| WWOX | 16 | 78,691,132 | RGS7 | 1 | 240,938,417 |
| RGS7 | 1 | 240,938,621 | CSMD1 | 8 | 3,230,841 |
| ATRNL1 | 10 | 117,029,233 | GPC6 | 13 | 94,080,565 |
| GPC6 | 13 | 94,084,293 | SLC35F3 | 1 | 234,178,299 |
| SULT4A1 | 22 | 44,241,285 | CACNA2D3 | 3 | 54,651,305 |
| UNC5C | 4 | 96,314,874 | TMEM106B | 7 | 12,272,116 |
| ANK3 | 10 | 61,789,753 | KCNN3 | 1 | 154,833,978 |
| CERKL | 2 | 182,403,387 | DGKB | 7 | 14,276,741 |
| HS6ST3 | 13 | 97,489,905 | PSD3 | 8 | 18,782,224 |
| MAGI2 | 7 | 78,658,175 | DOK6 | 18 | 67,133,967 |
| NAV3 | 12 | 78,240,316 | PDE10A | 6 | 165,800,773 |
| CAMKMT | 2 | 44,728,418 | ATP2B2 | 3 | 10,370,486 |
| RABGEF1 | 7 | 66,276,312 | DOK6 | 18 | 67,141,451 |
| CSMD1 | 8 | 3,485,004 | CLASP2 | 3 | 33,760,037 |
| DGKB | 7 | 14,198,845 | NKAIN2 | 6 | 124,912,456 |
| GRIN3A | 9 | 104,465,623 | LOC100505806 | 5 | 9,547,958 |
| LRP1B | 2 | 142,878,897 | SORBS1 | 10 | 97,118,644 |
| MTSS1 | 8 | 125,668,599 | THBS4 | 5 | 79,330,559 |
| MAML3 | 4 | 140,694,319 | MAGI2 | 7 | 78,843,289 |
| SGCZ | 8 | 14,143,813 | NALCN | 13 | 101,774,206 |
| KLF12 | 13 | 74,268,928 | KCNK10 | 14 | 88,795,203 |
| LOC100506128 | 1 | 177,704,242 | SORCS3 | 10 | 106,403,115 |
| SVEP1 | 9 | 113,131,354 | GPC5 | 13 | 93,279,684 |
| NTM | 11 | 131,778,518 | EXOC2 | 6 | 647,406 |
| VPS41 | 7 | 38,763,891 | PLCB1 | 20 | 8,865,006 |
| PLCB1 | 20 | 8,865,868 | PPM1H | 12 | 63,041,307 |
| NRG3 | 10 | 84,415,305 | DPP6 | 7 | 153,909,539 |
| PRODH | 22 | 18,912,678 | RYR2 | 1 | 237,825,673 |
| GRB10 | 7 | 50,809,771 | GRID2 | 4 | 94,259,545 |
| BMPR1B | 4 | 96,076,465 | UNC5C | 4 | 96,089,692 |
| CDH4 | 20 | 60,392,038 | LYN | 8 | 56,824,558 |
| CNTN4 | 3 | 2,465,091 | NALCN | 13 | 101,724,041 |
| SLIT2 | 4 | 20,286,220 | CDH4 | 20 | 60,392,016 |
| NALCN | 13 | 101,726,145 | ARHGAP31 | 3 | 119,137,912 |
| GRIA1 | 5 | 152,919,794 | HIATL1 | 9 | 97,223,294 |
| PSD3 | 8 | 18,645,147 | MACROD2 | 20 | 15,510,834 |
| LOC100616530 | 8 | 96,775,900 | BAG3 | 10 | 121,429,394 |
| NALCN | 13 | 101,737,731 | FHIT | 3 | 60,595,646 |
| ODZ2 | 5 | 167,304,609 | ODZ2 | 5 | 167,270,818 |
| TRPM3 | 9 | 73,502,424 | PARD3B | 2 | 205,593,602 |
| SGCZ | 8 | 14,789,538 | NEDD9 | 6 | 11,186,049 |
| FMN2 | 1 | 240,466,440 | ZNF169 | 9 | 97,064,273 |
| PARD3B | 2 | 205,619,609 | ANK2 | 4 | 114,279,674 |
| CDH4 | 20 | 60,308,635 | C15orf41 | 15 | 37,010,246 |
| S100PBP | 1 | 33,324,088 | BCL2L11 | 2 | 111,923,630 |
| SGCZ | 8 | 14,965,403 | VSNL1 | 2 | 17,720,520 |
| PTPRG | 3 | 61,610,728 | CDS1 | 4 | 85,572,374 |
| CAPZB, | 1 | 19,727,062 | CSMD1 | 8 | 4,501,890 |
| LOC644083 | | | | | |
| PARD3B | 2 | 205,606,092 | KCNB2 | 8 | 73,815,971 |
| ADAMTSL1 | 9 | 18,797,857 | TMEM181 | 6 | 159,051,186 |
| METTL21A | 2 | 208,489,101 | PTPRG | 3 | 61,608,761 |
| EPHB1 | 3 | 134,519,445 | ATP10A | 15 | 26,038,355 |
| NPAS3 | 14 | 33,618,223 | NALCN | 13 | 101,706,716 |
| EPHB1 | 3 | 134,509,337 | DAPK1 | 9 | 90,292,625 |
| SLC25A21 | 14 | 37,156,758 | DAPK1 | 9 | 90,293,359 |
| CNTNAP2 | 7 | 147,023,663 | VSNL1 | 2 | 17,747,999 |
| RGS7 | 1 | 241,241,733 | SYCP2 | 20 | 58,476,841 |
| CTNNA2 | 2 | 80,484,593 | MAGI1 | 3 | 65,383,903 |
| TRPM3 | 9 | 73,484,805 | NALCN | 13 | 101,732,146 |
| GRID2 | 4 | 93,236,404 | TSPAN9 | 12 | 3,394,098 |
| RAB36 | 22 | 23,505,804 | SCUBE1 | 22 | 43,703,729 |
| ARNTL | 11 | 13,297,925 | ARNTL | 11 | 13,298,485 |
| ARNTL | 11 | 13,298,519 | ARNTL | 11 | 13,298,687 |
| ARNTL | 11 | 13,298,750 | RC3H1 | 1 | 174,188,285 |
| DYNC1I1 | 7 | 95,725,936 | NXPH1 | 7 | 8,582,476 |
| ERC2 | 3 | 55,660,874 | GLP1R | 6 | 39,055,421 |
| CNTN4 | 3 | 2,730,859 | SYNE1 | 6 | 152,539,054 |
| ARNTL | 11 | 13,318,587 | CDH7 | 18 | 63,491,797 |
| RYR2 | 1 | 237,550,323 | HYDIN | 16 | 70,891,640 |
| EYA4 | 6 | 133,678,974 | RHOG | 11 | 3,855,859 |
| PES1 | 22 | 30,977,353 | DPYSL5 | 2 | 27,152,874 |
| PRKG1 | 10 | 53,295,318 | DLGAP1 | 18 | 3,651,423 |
| NKAIN2 | 6 | 124,196,534 | DLGAP1 | 18 | 3,656,488 |
| ADAMTS19 | 5 | 128,823,842 | NBEA | 13 | 35,827,215 |
| UTRN | 6 | 144,852,201 | EXOC2 | 6 | 604,461 |
| CTNNA3, | 10 | 68,685,929 | ZNF169 | 9 | 97,064,439 |
| LRRTM3 | | | | | |
| CAPZB, | 1 | 19,727,145 | ODZ2 | 5 | 167,251,985 |
| LOC644083 | | | | | |
| ADAMTSL1 | 9 | 18,789,128 | MTSS1 | 8 | 125,742,166 |
| LIMCH1 | 4 | 41,387,463 | SRRM4 | 12 | 119,419,603 |
| CACNA2D1 | 7 | 81,614,857 | NALCN | 13 | 101,720,300 |
| DAPK1 | 9 | 90,297,750 | PACRG | 6 | 163,213,454 |
| UTRN | 6 | 144,817,792 | KCNMA1 | 10 | 78,806,647 |
| MAGI2 | 7 | 78,125,648 | SDK1 | 7 | 3,726,122 |
| SGCZ | 8 | 14,846,341 | SEMA3E | 7 | 82,995,006 |
| SDK1 | 7 | 3,344,401 | ODZ2 | 5 | 167,236,746 |
| DLG2 | 11 | 83,456,191 | NCKAP5 | 2 | 133,694,153 |
| PLCG2 | 16 | 81,906,377 | FGF5 | 4 | 81,207,963 |
| ADAMTSL1 | 9 | 18,799,412 | INPP4A | 2 | 99,207,180 |
| DGKI | 7 | 137,072,931 | TBC1D1 | 4 | 38,007,099 |
| TSPAN11 | 12 | 31,145,084 | GABRP | 5 | 170,207,363 |
| FBXL17 | 5 | 107,352,294 | GRM8 | 7 | 126,884,450 |
| F5 | 1 | 169,511,878 | FBXL17 | 5 | 107,349,711 |
| GABBR2 | 9 | 101,052,858 | ULK1 | 12 | 132,378,133 |
| SHC3 | 9 | 91,640,058 | PLD5 | 1 | 242,675,820 |
| KLHL29 | 2 | 23,766,136 | DOK6 | 18 | 67,513,581 |
| MSI2 | 17 | 55,536,291 | ODZ2 | 5 | 167,304,210 |
| KCNMA1 | 10 | 78,726,699 | PLA2R1 | 2 | 160,920,122 |
| ODZ2 | 5 | 167,278,330 | SLC35F3 | 1 | 234,424,751 |
| CNTN5 | 11 | 99,690,286 | UTRN | 6 | 144,822,128 |
| LIMCH1 | 4 | 41,376,680 | INSC | 11 | 15,243,059 |
| KLHL29 | 2 | 23,769,972 | KCNK9 | 8 | 140,632,384 |
| SDK1 | 7 | 3,343,382 | CDH13 | 16 | 83,719,772 |
| RYR3 | 15 | 33,731,261 | ODZ2 | 5 | 167,177,952 |
| ODZ2 | 5 | 167,210,121 | CDH13 | 16 | 83,727,444 |
| LOC100289130, | 3 | 49,395,757 | CTNNA2 | 2 | 79,991,329 |
| GPX1 | | | | | |
| LIMCH1 | 4 | 41,380,276 | PARK2 | 6 | 161,971,805 |
| SCD5 | 4 | 83,595,238 | RAB11FIP4 | 17 | 29,863,723 |
| PDE4D | 5 | 59,032,179 | CDS1 | 4 | 85,571,339 |
| ATF6 | 1 | 161,735,397 | RORA | 15 | 60,878,030 |
| RARB | 3 | 25,512,768 | TPH2 | 12 | 72,355,179 |
| ATP10A | 15 | 26,094,520 | MICAL2 | 11 | 12,190,617 |
| LRP1B | 2 | 142,878,411 | KCNJ3 | 2 | 155,622,177 |
| C12orf5 | 12 | 4,462,161 | CDH13 | 16 | 83,492,421 |
| SYT13 | 11 | 45,276,308 | ARHGAP19- | 10 | 98,946,244 |
| | | | SLIT1 | | |
| CDH13 | 16 | 83,621,093 | NCAM2 | 21 | 22,381,606 |
| DPYSL5 | 2 | 27,171,245 | DPYSL5 | 2 | 27,172,069 |
| PRUNE2 | 9 | 79,318,998 | PRUNE2 | 9 | 79,320,640 |
| CPLX2 | 5 | 175,309,540 | C15orf41 | 15 | 36,989,469 |
| CTNND2 | 5 | 11,168,613 | MAGI1 | 3 | 65,376,512 |
| FHIT | 3 | 59,998,294 | CDH13 | 16 | 82,697,593 |
| TRPC4 | 13 | 38,444,350 | OPCML | 11 | 132,700,012 |
| FSTL5 | 4 | 162,318,470 | GIGYF2 | 2 | 233,641,924 |
| DLG2 | 11 | 83,463,013 | SYT13 | 11 | 45,309,171 |
| SYT13 | 11 | 45,309,202 | UBL3 | 13 | 30,343,129 |
| UBL3 | 13 | 30,418,719 | PTPRG | 3 | 61,583,771 |
| PTPRG | 3 | 61,590,517 | SPIB | 19 | 50,931,964 |
| NELL1 | 11 | 21,426,570 | CDH23 | 10 | 73,199,595 |
| VSNL1 | 2 | 17,754,316 | CTBP2 | 10 | 126,715,154 |
| NCKAP5 | 2 | 133,696,308 | NAV3 | 12 | 78,519,147 |
| NRXN3 | 14 | 79,890,456 | NRXN3 | 14 | 79,681,303 |
| KYNU | 2 | 143,746,494 | DENND5B | 12 | 31,536,540 |
| CACNA2D3 | 3 | 54,633,739 | USH2A | 1 | 216,597,759 |
| PSD3 | 8 | 18,634,283 | NLGN1 | 3 | 173,438,357 |
| FMN2 | 1 | 240,496,759 | CSMD1 | 8 | 4,492,255 |
| MAGI2 | 7 | 79,081,703 | KIAA1797 | 9 | 20,663,063 |
| BMP7 | 20 | 55,745,485 | PCLO | 7 | 82,516,893 |
| CDH4 | 20 | 60,512,369 | GALNTL4 | 11 | 11,525,323 |
| CACNB2 | 10 | 18,688,883 | SDK1 | 7 | 3,336,778 |
| NRXN3 | 14 | 79,657,049 | NCKAP5 | 2 | 133,541,107 |
| F5 | 1 | 169,481,950 | CDH13 | 16 | 83,677,493 |
| ROBO1 | 3 | 79,639,575 | LRRC4C | 11 | 40,181,102 |
| NRXN3 | 14 | 80,247,360 | MBP | 18 | 74,691,225 |
| MICAL2 | 11 | 12,191,487 | MIER1 | 1 | 67,451,487 |
| WWOX | 16 | 79,091,392 | PTGS2 | 1 | 186,650,321 |
| SH3GL3 | 15 | 84,215,428 | ABCA13 | 7 | 48,392,771 |
| TBXAS1 | 7 | 139,681,804 | RYR2 | 1 | 237,650,289 |
| DLG2 | 11 | 83,577,954 | SLC22A23 | 6 | 3,307,422 |
| COL22A1 | 8 | 139,869,353 | DOCK1 | 10 | 129,249,662 |
| CDH23 | 10 | 73,163,995 | ADCY8 | 8 | 132,053,412 |
| MIR1270-1 | 19 | 20,506,989 | AKAP6 | 14 | 33,293,531 |
| ARHGEF10 | 8 | 1,906,630 | SLCO3A1 | 15 | 92,529,323 |
| CTBP2 | 10 | 126,741,854 | CCDC88C | 14 | 91,771,625 |
| MTO1 | 6 | 74,171,467 | PARK2 | 6 | 162,291,767 |
| DLC1 | 8 | 12,942,342 | CNTN4 | 3 | 2,876,258 |
| MAGI1 | 3 | 65,554,067 | DOK6 | 18 | 67,192,125 |
| CACNA1E | 1 | 181,768,985 | SLC1A3 | 5 | 36,667,579 |
| C10orf112 | 10 | 19,678,497 | COL6A3 | 2 | 238,233,410 |
| SNRNP27 | 2 | 70,131,791 | FHIT | 3 | 59,761,158 |
| MBP | 18 | 74,724,257 | WWOX | 16 | 78,327,521 |
| PTPRT | 20 | 40,791,519 | FBXL2 | 3 | 33,348,480 |
| PCSK6 | 15 | 102,024,136 | PCSK6 | 15 | 102,028,088 |
| FMN2 | 1 | 240,472,692 | TPH2 | 12 | 72,412,572 |
| COL6A3 | 2 | 238,262,021 | LIMCH1 | 4 | 41,378,712 |
| TRDN | 6 | 123,776,326 | CRISPLD2 | 16 | 84,942,638 |
| CNTNAP2 | 7 | 147,809,195 | MMP16 | 8 | 89,075,226 |
| PDE1C | 7 | 32,025,543 | CTNND2 | 5 | 10,984,056 |
| MIOX | 22 | 50,928,340 | MACROD2 | 20 | 15,438,981 |
| PTPRN2 | 7 | 157,870,786 | TMC8 | 17 | 76,137,589 |
| COL4A4 | 2 | 228,014,602 | GAS7 | 17 | 9,943,867 |
| PCDH17 | 13 | 58,258,094 | TRPM6 | 9 | 77,350,356 |
| ZXDC | 3 | 126,156,660 | STK10 | 5 | 171,471,045 |
| GRK5 | 10 | 121,161,798 | KCNJ3 | 2 | 155,553,275 |
| CELSR3 | 3 | 48,675,064 | TSPAN5 | 4 | 99,550,164 |
| GRID2 | 4 | 93,304,682 | SH3GL3 | 15 | 84,178,640 |
| TMEM132B | 12 | 126,140,066 | LRRC4C | 11 | 40,227,380 |
| SHROOM3 | 4 | 77,441,688 | NAALADL2 | 3 | 175,521,611 |
| CELSR3 | 3 | 48,697,654 | LRP1B | 2 | 142,835,731 |

### B. Markers in Linkage Disequilibrium (LD)

Linkage disequilibrium (LD) is a measure of the degree of association between alleles in a population. One of skill in the art will appreciate that alleles involving markers in LD with the polymorphisms described herein can also be used in a similar manner to those described herein. Methods of calculating LD are known in the art (see, e.g., Morton et al., 2001; Tapper et al., 2005; Maniatis et al., 2002). Thus, in some cases, the methods can include analysis of polymorphisms that are in LD with a polymorphism described herein. Methods are known in the art for identifying such polymorphisms; for example, the International HapMap Project provides a public database that can be used, see hapmap.org, as well as The International HapMap Consortium (2003) and The International HapMap Consortium (2005). Generally, it will be desirable to use a HapMap constructed using data from individuals who share ethnicity with the subject. For example, a HapMap for Caucasians would ideally be used to identify markers in LD with an exemplary marker described herein for use in genotyping a subject of Caucasian descent.

Alternatively, methods described herein can include analysis of polymorphisms that show a correlation coefficient (r²) of value >0.5 with the markers described herein. Results can be obtained from on line public resources such as HapMap.org on the World Wide Web. The correlation coefficient is a measure of LD, and reflects the degree to which alleles at two loci (for example, two SNPs) occur together, such that an allele at one SNP position can predict the correlated allele at a second SNP position, in the case where r² is >0.5.

### C. Identifying Additional Genetic Markers

In general, genetic markers can be identified using any of a number of methods well known in the art. For example, numerous polymorphisms in the regions described herein are known to exist and are available in public databases, which can be searched using methods and algorithms known in the art. Alternately, polymorphisms can be identified by sequencing either genomic DNA or cDNA in the region in which it is desired to find a polymorphism. According to one approach, primers are designed to amplify such a region, and DNA from a subject is obtained and amplified. The DNA is sequenced, and the sequence (referred to as a “subject sequence” or “test sequence”) is compared with a reference sequence, which can represent the “normal” or “wild type” sequence, or the “affected” sequence. In some embodiments, a reference sequence can be from, for example, the human draft genome sequence, publicly available in various databases, or a sequence deposited in a database such as GenBank. In some embodiments, the reference sequence is a composite of ethnically diverse individuals.

In general, if sequencing reveals a difference between the sequenced region and the reference sequence, a polymorphism has been identified. The fact that a difference in nucleotide sequence is identified at a particular site is what determines that a polymorphism exists at that site.

In most instances, particularly in the case of SNPs, only two polymorphic variants will exist at any location. However, in the case of SNPs, up to four variants may exist since there are four naturally occurring nucleotides in DNA. Other polymorphisms, such as insertions and deletions, may have more than four alleles.

In some embodiments, the methods include determining the presence or absence of one or more other markers that are or may be associated with treatment response, e.g., in one or more genes, e.g., as described in WO 2009/092032, WO 2009/089120, WO 2009/082743, US2006/0177851, or US2009/0012371, incorporated herein in their entirety. See also, e.g., OMIM entry no. 181500 (SCZD).

### D. Methods of Determining the Identity of a Subject's Response Allele

The methods described herein include determining the identity, e.g., the specific nucleotide, presence or absence, of alleles associated with a predicted response to a treatment for an SSD, e.g., SZ. In some embodiments, a predicted response to a method of treating an SSD is determined by detecting the presence of an identical allele in both the subject and an individual with a known response to a method of treating an SSD, e.g., in an unrelated reference subject or a first or second-degree relation of the subject, and, in some cases, the absence of the allele in an reference individual having a known but opposite response. Thus the methods can include obtaining and analyzing a sample from a suitable reference individual. Samples that are suitable for use in the methods described herein contain genetic material, e.g., genomic DNA (gDNA). Genomic DNA is typically extracted from biological samples such as blood or mucosal scrapings of the lining of the mouth, but can be extracted from other biological samples including urine or expectorant. The sample itself will typically include nucleated cells (e.g., blood or buccal cells) or tissue removed from the subject. The subject can be an adult, child, fetus, or embryo. In some embodiments, the sample is obtained prenatally, either from a fetus or embryo or from the mother (e.g., from fetal or embryonic cells in the maternal circulation). Methods and reagents are known in the art for obtaining, processing, and analyzing samples. In some embodiments, the sample is obtained with the assistance of a health care provider, e.g., to draw blood. In some embodiments, the sample is obtained without the assistance of a health care provider, e.g., where the sample is obtained non-invasively, such as a sample comprising buccal cells that is obtained using a buccal swab or brush, or a mouthwash sample.

In some cases, a biological sample may be processed for DNA isolation. For example, DNA in a cell or tissue sample can be separated from other components of the sample. Cells can be harvested from a biological sample using standard techniques known in the art. For example, cells can be harvested by centrifuging a cell sample and resuspending the pelleted cells. The cells can be resuspended in a buffered solution such as phosphate-buffered saline (PBS). After centrifuging the cell suspension to obtain a cell pellet, the cells can be lysed to extract DNA, e.g., gDNA. See, e.g., Ausubel et al. (2003). The sample can be concentrated and/or purified to isolate DNA. All samples obtained from a subject, including those subjected to any sort of further processing, are considered to be obtained from the subject. Routine methods can be used to extract genomic DNA from a biological sample, including, for example, phenol extraction. Alternatively, genomic DNA can be extracted with kits such as the QIAamp® Tissue Kit (Qiagen, Chatsworth, Calif.) and the Wizard® Genomic DNA purification kit (Promega). Non-limiting examples of sources of samples include urine, blood, and tissue.

The presence or absence of an allele or genotype associated with a predicted response to treatment for an SPD (e.g., SZ) as described herein can be determined using methods known in the art. For example, gel electrophoresis, capillary electrophoresis, size exclusion chromatography, sequencing, and/or arrays can be used to detect the presence or absence of specific response alleles. Amplification of nucleic acids, where desirable, can be accomplished using methods known in the art, e.g., PCR. In one example, a sample (e.g., a sample comprising genomic DNA), is obtained from a subject. The DNA in the sample is then examined to determine the identity of an allele as described herein, i.e., by determining the identity of one or more alleles associated with a selected response. The identity of an allele can be determined by any method described herein, e.g., by sequencing or by hybridization of the gene in the genomic DNA, RNA, or cDNA to a nucleic acid probe, e.g., a DNA probe (which includes cDNA and oligonucleotide probes) or an RNA probe. The nucleic acid probe can be designed to specifically or preferentially hybridize with a particular polymorphic variant.

Other methods of nucleic acid analysis can include direct manual sequencing (Church and Gilbert, 1988; Sanger et al., 1977; U.S. Pat. No. 5,288,644); automated fluorescent sequencing; single-stranded conformation polymorphism assays (SSCP) (Schafer et al., 1995); clamped denaturing gel electrophoresis (CDGE); two-dimensional gel electrophoresis (2DGE or TDGE); conformational sensitive gel electrophoresis (CSGE); denaturing gradient gel electrophoresis (DGGE) (Sheffield et al., 1989); denaturing high performance liquid chromatography (DHPLC, Underhill et al., 1997); infrared matrix-assisted laser desorption/ionization (IR-MALDI) mass spectrometry (WO 99/57318); mobility shift analysis (Orita et al., 1989); restriction enzyme analysis (Flavell et al., 1978; Geever et al., 1981); quantitative real-time PCR (Raca et al., 2004); heteroduplex analysis; chemical mismatch cleavage (CMC) (Cotton et al., 1985); RNase protection assays (Myers et al., 1985); use of polypeptides that recognize nucleotide mismatches, e.g., E. coli mutS protein; allele-specific PCR, and combinations of such methods. See, e.g., U.S. Patent Publication No. 2004/0014095, which is incorporated herein by reference in its entirety. Sequence analysis can also be used to detect specific polymorphic variants. For example, polymorphic variants can be detected by sequencing exons, introns, 5′ untranslated sequences, or 3′ untranslated sequences. A sample comprising DNA or RNA is obtained from the subject. PCR or other appropriate methods can be used to amplify a portion encompassing the polymorphic site, if desired. The sequence is then ascertained, using any standard method, and the presence of a polymorphic variant is determined Real-time pyrophosphate DNA sequencing is yet another approach to detection of polymorphisms and polymorphic variants (Alderborn et al., 2000). Additional methods include, for example, PCR amplification in combination with denaturing high performance liquid chromatography (dHPLC) (Underhill et al., 1997).

In order to detect polymorphisms and/or polymorphic variants, it may be desirable to amplify a portion of genomic DNA (gDNA) encompassing the polymorphic site. Such regions can be amplified and isolated by PCR using oligonucleotide primers designed based on genomic and/or cDNA sequences that flank the site. PCR refers to procedures in which target nucleic acid (e.g., genomic DNA) is amplified in a manner similar to that described in U.S. Pat. No. 4,683,195, and subsequent modifications of the procedure described therein. Generally, sequence information from the ends of the region of interest or beyond are used to design oligonucleotide primers that are identical or similar in sequence to opposite strands of a potential template to be amplified. See e.g., PCR Primer: A Laboratory Manual, Dieffenbach and Dveksler, (Eds.); McPherson et al., 2000; Mattila et al., 1991; Eckert et al., 1991; PCR (eds. McPherson et al., IRL Press, Oxford); and U.S. Pat. No. 4,683,202. Other amplification methods that may be employed include the ligase chain reaction (LCR) (Wu and Wallace, 1989; Landegren et al., 1988), transcription amplification (Kwoh et al., 1989), self-sustained sequence replication (Guatelli et al., 1990), and nucleic acid based sequence amplification (NASBA). Guidelines for selecting primers for PCR amplification are well known in the art. See, e.g., McPherson et al. (2000). A variety of computer programs for designing primers are available, e.g., ‘Oligo’ (National Biosciences, Inc, Plymouth Minn.), MacVector (Kodak/IBI), and the GCG suite of sequence analysis programs (Genetics Computer Group, Madison, Wis. 53711).

In some cases, PCR conditions and primers can be developed that amplify a product only when the variant allele is present or only when the wild type allele is present (MSPCR or allele-specific PCR). For example, patient DNA and a control can be amplified separately using either a wild type primer or a primer specific for the variant allele. Each set of reactions is then examined for the presence of amplification products using standard methods to visualize the DNA. For example, the reactions can be electrophoresed through an agarose gel and the DNA visualized by staining with ethidium bromide or other DNA intercalating dye. In DNA samples from heterozygous patients, reaction products would be detected in each reaction.

Real-time quantitative PCR can also be used to determine copy number. Quantitative PCR permits both detection and quantification of specific DNA sequence in a sample as an absolute number of copies or as a relative amount when normalized to DNA input or other normalizing genes. A key feature of quantitative PCR is that the amplified DNA product is quantified in real-time as it accumulates in the reaction after each amplification cycle. Methods of quantification can include the use of fluorescent dyes that intercalate with double-stranded DNA, and modified DNA oligonucleotide probes that fluoresce when hybridized with a complementary DNA. Methods of quantification can include determining the intensity of fluorescence for fluorescently tagged molecular probes attached to a solid surface such as a microarray.

The first report of extensive copy number variation (CNV) in the human genome used intensity analysis of microarray data to document numerous examples of genes that vary in copy number (Redon et al., 2006). Subsequent studies have shown that certain copy number variants are associated with complex genetic diseases such as SZ (Walsh et al., 2008; Stone et al., 2008).

In some embodiments, a peptide nucleic acid (PNA) probe can be used instead of a nucleic acid probe in the hybridization methods described above. PNA is a DNA mimetic with a peptide-like, inorganic backbone, e.g., N-(2-aminoethyl)glycine units, with an organic base (A, G, C, T or U) attached to the glycine nitrogen via a methylene carbonyl linker (see, e.g., Nielsen et al., 1994). The PNA probe can be designed to specifically hybridize to a nucleic acid comprising a polymorphic variant.

In some cases, allele-specific oligonucleotides can also be used to detect the presence of a polymorphic variant. For example, polymorphic variants can be detected by performing allele-specific hybridization or allele-specific restriction digests. Allele specific hybridization is an example of a method that can be used to detect sequence variants, including complete genotypes of a subject (e.g., a mammal such as a human). See Stoneking et al., 1991; Prince et al., 2001. An “allele-specific oligonucleotide” (also referred to herein as an “allele-specific oligonucleotide probe”) is an oligonucleotide that is specific for particular a polymorphism can be prepared using standard methods (see, Ausubel et al., 2003). Allele-specific oligonucleotide probes typically can be approximately 10-50 base pairs, preferably approximately 15-30 base pairs, that specifically hybridizes to a nucleic acid region that contains a polymorphism. Hybridization conditions are selected such that a nucleic acid probe can specifically bind to the sequence of interest, e.g., the variant nucleic acid sequence. Such hybridizations typically are performed under high stringency as some sequence variants include only a single nucleotide difference. In some cases, dot-blot hybridization of amplified oligonucleotides with allele-specific oligonucleotide (ASO) probes can be performed. See, for example, Saiki et al., 1986.

In some embodiments, allele-specific restriction digest analysis can be used to detect the existence of a polymorphic variant of a polymorphism, if alternate polymorphic variants of the polymorphism result in the creation or elimination of a restriction site. Allele-specific restriction digests can be performed in the following manner. A sample containing genomic DNA is obtained from the individual and genomic DNA is isolated for analysis. For nucleotide sequence variants that introduce a restriction site, restriction digest with the particular restriction enzyme can differentiate the alleles. In some cases, polymerase chain reaction (PCR) can be used to amplify a region comprising the polymorphic site, and restriction fragment length polymorphism analysis is conducted (see, Ausubel et al., 2003). The digestion pattern of the relevant DNA fragment indicates the presence or absence of a particular polymorphic variant of the polymorphism and is therefore indicative of the subject's response allele. For sequence variants that do not alter a common restriction site, mutagenic primers can be designed that introduce a restriction site when the variant allele is present or when the wild type allele is present. For example, a portion of a nucleic acid can be amplified using the mutagenic primer and a wild type primer, followed by digest with the appropriate restriction endonuclease.

In some embodiments, fluorescence polarization template-directed dye-terminator incorporation (FP-TDI) is used to determine which of multiple polymorphic variants of a polymorphism is present in a subject (Chen et al., 1999). Rather than involving use of allele-specific probes or primers, this method employs primers that terminate adjacent to a polymorphic site, so that extension of the primer by a single nucleotide results in incorporation of a nucleotide complementary to the polymorphic variant at the polymorphic site.

In some cases, DNA containing an amplified portion may be dot-blotted, using standard methods (see Ausubel et al., 2003), and the blot contacted with the oligonucleotide probe. The presence of specific hybridization of the probe to the DNA is then detected. Specific hybridization of an allele-specific oligonucleotide probe (specific for a polymorphic variant indicative of a predicted response to a method of treating an SSD) to DNA from the subject is indicative of a subject's response allele.

The methods can include determining the genotype of a subject with respect to both copies of the polymorphic site present in the genome (i.e., both alleles). For example, the complete genotype may be characterized as −/−, as −/+, or as +/+, where a minus sign indicates the presence of the reference or wild type sequence at the polymorphic site, and the plus sign indicates the presence of a polymorphic variant other than the reference sequence. If multiple polymorphic variants exist at a site, this can be appropriately indicated by specifying which ones are present in the subject. Any of the detection means described herein can be used to determine the genotype of a subject with respect to one or both copies of the polymorphism present in the subject's genome.

Methods of nucleic acid analysis to detect polymorphisms and/or polymorphic variants can include, e.g., microarray analysis. Hybridization methods, such as Southern analysis, Northern analysis, or in situ hybridizations, can also be used (see, Ausubel et al., 2003). To detect microdeletions, fluorescence in situ hybridization (FISH) using DNA probes that are directed to a putatively deleted region in a chromosome can be used. For example, probes that detect all or a part of a microsatellite marker can be used to detect microdeletions in the region that contains that marker.

In some embodiments, it is desirable to employ methods that can detect the presence of multiple polymorphisms (e.g., polymorphic variants at a plurality of polymorphic sites) in parallel or substantially simultaneously. Oligonucleotide arrays represent one suitable means for doing so. Other methods, including methods in which reactions (e.g., amplification, hybridization) are performed in individual vessels, e.g., within individual wells of a multi-well plate or other vessel may also be performed so as to detect the presence of multiple polymorphic variants (e.g., polymorphic variants at a plurality of polymorphic sites) in parallel or substantially simultaneously according to the methods provided herein.

Nucleic acid probes can be used to detect and/or quantify the presence of a particular target nucleic acid sequence within a sample of nucleic acid sequences, e.g., as hybridization probes, or to amplify a particular target sequence within a sample, e.g., as a primer. Probes have a complimentary nucleic acid sequence that selectively hybridizes to the target nucleic acid sequence. In order for a probe to hybridize to a target sequence, the hybridization probe must have sufficient identity with the target sequence, i.e., at least 70% (e.g., 80%, 90%, 95%, 98% or more) identity to the target sequence. The probe sequence must also be sufficiently long so that the probe exhibits selectivity for the target sequence over non-target sequences. For example, the probe will be at least 20 (e.g., 25, 30, 35, 50, 100, 200, 300, 400, 500, 600, 700, 800, 900 or more) nucleotides in length. In some embodiments, the probes are not more than 30, 50, 100, 200, 300, 500, 750, or 1000 nucleotides in length. Probes are typically about 20 to about 1×10⁶ nucleotides in length. Probes include primers, which generally refers to a single-stranded oligonucleotide probe that can act as a point of initiation of template-directed DNA synthesis using methods such as PCR (polymerase chain reaction), LCR (ligase chain reaction), etc., for amplification of a target sequence.

The probe can be a test probe such as a probe that can be used to detect polymorphisms in a region described herein (e.g., an allele associated with treatment response as described herein). In some embodiments, the probe can bind to another marker sequence associated with SZ, SPD, or SD as described herein or known in the art.

Control probes can also be used. For example, a probe that binds a less variable sequence, e.g., repetitive DNA associated with a centromere of a chromosome, can be used as a control. Probes that hybridize with various centromeric DNA and locus-specific DNA are available commercially, for example, from Vysis, Inc. (Downers Grove, Ill.), Molecular Probes, Inc. (Eugene, Oreg.), or from Cytocell (Oxfordshire, UK). Probe sets are available commercially such from Applied Biosystems, e.g., the Assays-on-Demand SNP kits Alternatively, probes can be synthesized, e.g., chemically or in vitro, or made from chromosomal or genomic DNA through standard techniques. For example, sources of DNA that can be used include genomic DNA, cloned DNA sequences, somatic cell hybrids that contain one, or a part of one, human chromosome along with the normal chromosome complement of the host, and chromosomes purified by flow cytometry or microdissection. The region of interest can be isolated through cloning, or by site-specific amplification via the polymerase chain reaction (PCR). See, for example, Nath and Johnson, (1998); Wheeless et al., (1994); U. S. Pat. No. 5,491,224.

In some embodiments, the probes are labeled, e.g., by direct labeling, with a fluorophore, an organic molecule that fluoresces after absorbing light of lower wavelength/higher energy. A directly labeled fluorophore allows the probe to be visualized without a secondary detection molecule. After covalently attaching a fluorophore to a nucleotide, the nucleotide can be directly incorporated into the probe with standard techniques such as nick translation, random priming, and PCR labeling. Alternatively, deoxycytidine nucleotides within the probe can be transaminated with a linker. The fluorophore then is covalently attached to the transaminated deoxycytidine nucleotides. See, e.g., U.S. Pat. No. 5,491,224.

Fluorophores of different colors can be chosen such that each probe in a set can be distinctly visualized. For example, a combination of the following fluorophores can be used: 7-amino-4-methylcoumarin-3-acetic acid (AMCA), TEXAS RED™ (Molecular Probes, Inc., Eugene, Oreg.), 5-(and -6)-carboxy-X-rhodamine, lissamine rhodamine B, 5-(and −6)-carboxyfluorescein, fluorescein-5-isothiocyanate (FITC), 7-diethylaminocoumarin-3-carboxylic acid, tetramethylrhodamine-5-(and -6)-isothiocyanate, 5-(and -6)-carboxytetramethylrhodamine, 7-hydroxycoumarin-3-carboxylic acid, 6-[fluorescein 5-(and -6)-carboxamido]hexanoic acid, N-(4,4-difluoro-5,7-dimethyl-4-bora-3a,4a diaza-3-indacenepropionic acid, eosin-5-isothiocyanate, erythrosin-5-isothiocyanate, and CASCADE™ blue acetylazide (Molecular Probes, Inc., Eugene, Oreg.). Fluorescently labeled probes can be viewed with a fluorescence microscope and an appropriate filter for each fluorophore, or by using dual or triple band-pass filter sets to observe multiple fluorophores. See, for example, U.S. Pat. No. 5,776,688. Alternatively, techniques such as flow cytometry can be used to examine the hybridization pattern of the probes. Fluorescence-based arrays are also known in the art.

In other embodiments, the probes can be indirectly labeled with, e.g., biotin or digoxygenin, or labeled with radioactive isotopes such as ³²P and ³H. For example, a probe indirectly labeled with biotin can be detected by avidin conjugated to a detectable marker. For example, avidin can be conjugated to an enzymatic marker such as alkaline phosphatase or horseradish peroxidase. Enzymatic markers can be detected in standard colorimetric reactions using a substrate and/or a catalyst for the enzyme. Catalysts for alkaline phosphatase include 5-bromo-4-chloro-3-indolylphosphate and nitro blue tetrazolium. Diaminobenzoate can be used as a catalyst for horseradish peroxidase.

In another aspect, this document features arrays that include a substrate having a plurality of addressable areas, and methods of using them. At least one area of the plurality includes a nucleic acid probe that binds specifically to a sequence comprising a polymorphism listed in Table A (or Tables 1-10), and can be used to detect the absence or presence of said polymorphism, e.g., one or more SNPs, microsatellites, minisatellites, or indels, as described herein, to determine a response allele. For example, the array can include one or more nucleic acid probes that can be used to detect a polymorphism listed in Table A or Tables 1-10. In some embodiments, the array further includes at least one area that includes a nucleic acid probe that can be used to specifically detect another marker associated with a predicted response to a method of treating an SSD (e.g., SZ), as described herein. In some embodiments, the probes are nucleic acid capture probes.

Generally, microarray hybridization is performed by hybridizing a nucleic acid of interest (e.g., a nucleic acid encompassing a polymorphic site) with the array and detecting hybridization using nucleic acid probes. In some cases, the nucleic acid of interest is amplified prior to hybridization. Hybridization and detecting are generally carried out according to standard methods. See, e.g., PCT Application Nos. WO 92/10092 and WO 95/11995, and U.S. Pat. No. 5,424,186. For example, the array can be scanned to determine the position on the array to which the nucleic acid hybridizes. The hybridization data obtained from the scan is typically in the form of fluorescence intensities as a function of location on the array.

Arrays can be formed on substrates fabricated with materials such as paper, glass, plastic (e.g., polypropylene, nylon, or polystyrene), polyacrylamide, nitrocellulose, silicon, optical fiber, or any other suitable solid or semisolid support, and can be configured in a planar (e.g., glass plates, silicon chips) or three dimensional (e.g., pins, fibers, beads, particles, microtiter wells, capillaries) configuration. Methods for generating arrays are known in the art and include, e.g., photolithographic methods (see, e.g., U.S. Pat. Nos. 5,143,854; 5,510,270; and 5,527,681), mechanical methods (e.g., directed-flow methods as described in U.S. Pat. No. 5,384,261), pin-based methods (e.g., as described in U.S. Pat. No. 5,288,514), and bead-based techniques (e.g., as described in PCT US/93/04145). The array typically includes oligonucleotide hybridization probes capable of specifically hybridizing to different polymorphic variants. Oligonucleotide probes that exhibit differential or selective binding to polymorphic sites may readily be designed by one of ordinary skill in the art. For example, an oligonucleotide that is perfectly complementary to a sequence that encompasses a polymorphic site (i.e., a sequence that includes the polymorphic site, within it or at one end) will generally hybridize preferentially to a nucleic acid comprising that sequence, as opposed to a nucleic acid comprising an alternate polymorphic variant.

Oligonucleotide probes forming an array may be attached to a substrate by any number of techniques, including, without limitation, (i) in situ synthesis (e.g., high-density oligonucleotide arrays) using photolithographic techniques; (ii) spotting/printing at medium to low density on glass, nylon or nitrocellulose; (iii) by masking, and (iv) by dot-blotting on a nylon or nitrocellulose hybridization membrane. Oligonucleotides can be immobilized via a linker, including by covalent, ionic, or physical linkage. Linkers for immobilizing nucleic acids and polypeptides, including reversible or cleavable linkers, are known in the art. See, for example, U.S. Pat. No. 5,451,683 and WO98/20019. Alternatively, oligonucleotides can be non-covalently immobilized on a substrate by hybridization to anchors, by means of magnetic beads, or in a fluid phase such as in microtiter wells or capillaries Immobilized oligonucleotide probes are typically about 20 nucleotides in length, but can vary from about 10 nucleotides to about 1000 nucleotides in length.

Arrays can include multiple detection blocks (i.e., multiple groups of probes designed for detection of particular polymorphisms). Such arrays can be used to analyze multiple different polymorphisms. Detection blocks may be grouped within a single array or in multiple, separate arrays so that varying conditions (e.g., conditions optimized for particular polymorphisms) may be used during the hybridization. For example, it may be desirable to provide for the detection of those polymorphisms that fall within G-C rich stretches of a genomic sequence, separately from those falling in A-T rich segments. General descriptions of using oligonucleotide arrays for detection of polymorphisms can be found, for example, in U.S. Pat. Nos. 5,858,659 and 5,837,832. In addition to oligonucleotide arrays, cDNA arrays may be used similarly in certain embodiments.

The methods described herein can include providing an array as described herein; contacting the array with a sample (e.g., all or a portion of genomic DNA that includes at least a portion of a human chromosome comprising a response allele) and/or optionally, a different portion of genomic DNA (e.g., a portion that includes a different portion of one or more human chromosomes), and detecting binding of a nucleic acid from the sample to the array. Optionally, the method includes amplifying nucleic acid from the sample, e.g., genomic DNA that includes a portion of a human chromosome described herein, and, optionally, a region that includes another region associated with a predicted response to a method of treating SZ, SD, or SPD, prior to or during contact with the array.

In some aspects, the methods described herein can include using an array that can ascertain differential expression patterns or copy numbers of one or more genes in samples from normal and affected individuals (see, e.g., Redon et al., 2006). For example, arrays of probes to a marker described herein can be used to measure polymorphisms between DNA from a subject having an SSD (e.g., SZ) and having a predicted response to a treatment for an SSD (e.g., SZ), and control DNA, e.g., DNA obtained from an individual that has SZ, SPD, or SD, and has a known response to a form of treatment for an SSD (e.g., SZ). Since the clones on the array contain sequence tags, their positions on the array are accurately known relative to the genomic sequence. Different hybridization patterns between DNA from an individual afflicted with an SSD (e.g., SZ) and DNA from a control individual at areas in the array corresponding to markers as described herein, and, optionally, one or more other regions associated with an SSD (e.g., SZ), are indicative of a predicted response to a treatment for an SSD (e.g., SZ). Methods for array production, hybridization, and analysis are described, e.g., in Snijders et al. (2001); Klein et al. (1999); Albertson et al. (2003); and Snijders et al. (2002).

In another aspect, this document provides methods of determining the absence or presence of a response allele associated with a predicted response to treatment for an SSD (e.g., SZ) as described herein, using an array described above. The methods can include providing a two dimensional array having a plurality of addresses, each address of the plurality being positionally distinguishable from each other address of the plurality having a unique nucleic acid capture probe, contacting the array with a first sample from a test subject who is has an SSD (e.g., SZ), and comparing the binding of the first sample with one or more references, e.g., binding of a sample from a subject who is known to have an SSD (e.g., SZ), and/or binding of a sample from a subject who has an SSD (e.g., SZ) and a known response to treatment for an SSD (e.g., SZ); and comparing the binding of the first sample with the binding of the second sample. In some embodiments, the methods can include contacting the array with a third sample from a cell or subject that does not have SZ; and comparing the binding of the first sample with the binding of the third sample. In some embodiments, the second and third samples are from first or second-degree relatives of the test subject. In the case of a nucleic acid hybridization, binding with a capture probe at an address of the plurality, can be detected by any method known in the art, e.g., by detection of a signal generated from a label attached to the nucleic acid.

### III. SCHIZOPHRENIA SPECTRUM DISORDERS

The methods described herein can be used to determine an individual predicted response to a method of treating a schizophrenia spectrum disorder (SSD). The SSDs include schizophrenia (SZ), schizotypal personality disorder (SPD), and schizoaffective disorder (SD). Methods for diagnosing SSDs are known in the art, see, e.g., the DSM-IV. See, e.g., WO 2009/092032, incorporated herein by reference.

### IV. METHODS OF SELECTING AND OPTIMIZING TREATMENT

In some embodiments, the methods described herein include the administration of one or more treatments, e.g., antipsychotic medications, to a person identified as having or being at risk of developing an SSD (e.g., SZ). The methods can also include selecting a treatment regimen for a subject who has an SSD or is determined to be at risk for developing an SSD (e.g., SZ), based upon the absence or presence of an allele or genotype associated with response as described herein. The determination of a treatment regimen can also be based upon the absence or presence of other risk factors, e.g., as known in the art or described herein. The methods can also include administering a treatment regimen selected by a method described to a subject who has or is at risk for developing an SSD (e.g., SZ) to thereby treat, reduce risk of developing, or delay further progression of the disease. A treatment regimen can include the administration of antipsychotic medications to a subject identified as having or at risk of developing an SSD (e.g., SZ) before the onset of any psychotic episodes.

In some embodiments, the approach described herein uses a multiple response allele algorithm rather than a single response allele or a group of single response alleles. Algorithms can be used to derive a single value that reflects disease status, prognosis, and/or response to treatment. Highly multiplexed tools can be used to simultaneously measure multiple parameters. An advantage of using such tools is that all results can be derived from the same sample and run under the same conditions at the same time. High-level pattern recognition approaches can be applied, and a number of tools are available, including clustering approaches such as hierarchical clustering, self-organizing maps, and supervised classification algorithms (e.g., support vector machines, k-nearest neighbors, and neural networks). The latter group of analytical approaches is likely to be of substantial clinical use. The basic method can include providing a biological sample (e.g., a blood sample) from a individual; determining the sequence of a group of response alleles in the sample; and using an algorithm to determine a SSD score.

Algorithms for determining an individual's disease status or response to treatment, for example, can be determined for any clinical condition. The algorithms provided herein can be mathematic functions containing multiple parameters that can be quantified using, for example, medical devices, clinical evaluation scores, or biological, chemical, or physical tests of biological samples. Each mathematical function can be a weight-adjusted expression of the parameters determined to be relevant to a selected clinical condition. Univariate and multivariate analyses can be performed on data collected for each marker using conventional statistical tools (e.g., not limited to: T-tests, PCA, LDA, or binary logistic regression). An algorithm can be applied to generate a set of diagnostic scores. The algorithms generally can be expressed in the format of Formula 1:

Diagnostic score=f(x1,x2,x3,x4,x5 . . . xn) (1).

The diagnostic score is a value that is the diagnostic or prognostic result, “f” is any mathematical function, “n” is any integer (e.g., an integer from 1 to 10,000), and x1, x2, x3, x4, x5 . . . xn are the “n” parameters that are, for example, measurements determined by medical devices, clinical evaluation scores, and/or test results for biological samples.

The parameters of an algorithm can be individually weighted. An example of such an algorithm is expressed in Formula 2:

Diagnostic score a1*x1−a2*x2−a3*x3 a4*x4−a5:*x5 (2).

Here, x1, x2, x3, x4, and x5 can be measurements determined by medical devices, clinical evaluation scores, and/or test results for biological samples (i, human biological samples), and a1, a2, a3, a4, and a5 are weight-adjusted factors for x1, x2, x3, x4, and x5, respectively.

A diagnostic score can be used to quantitatively define a medical condition or disease, or the effect of a medical treatment. In a more general form, multiple diagnostic scores Sm can be generated by applying multiple formulas to specific groupings of biomarker measurements, as illustrated in Formula 3:

Diagnostic Scores Sm=Fm(x1 . . . Xn) (3).

Multiple scores can be useful, for example, in the identification of specific types and subtypes of SSD. In some cases, the SSD is SZ. Multiple scores can also be parameters indicating patient treatment progress or the efficacy of the treatment selected. Diagnostic scores for subtypes of SSD may help aid in the selection or optimization of antipsychotics and other pharmaceuticals.

As used herein, the term “treat” or “treatment” is defined as the application or administration of a treatment regimen, e.g., a therapeutic agent or modality, to a subject, e.g., a patient. The subject can be a patient having an SSD (e.g., SZ), a symptom of an SSD (e.g., SZ), or at risk of developing (i.e., a predisposition toward) an SSD (e.g., SZ). The treatment can be to cure, heal, alleviate, relieve, alter, remedy, ameliorate, palliate, improve or affect an SSD (e.g., SZ), the symptoms of an SSD (e.g., SZ) or the predisposition toward an SSD (e.g., SZ). For example, a standard treatment regimen for schizophrenia is the administration of antipsychotic medications, which are typically antagonists acting at postsynaptic D2 dopamine receptors in the brain and can include neuroleptics and/or atypical antipsychotics. Antipsychotic medications substantially reduce the risk of relapse in the stable phase of illness. Currently accepted treatments for SZ are described in greater detail in the Practice Guideline for the Treatment of Patients With Schizophrenia American Psychiatric Association, Second Edition, American Psychiatric Association (2004), which is incorporated herein by reference in its entirety.

The methods of determining a treatment regimen and methods of treatment or prevention of SSDs as described herein can further include the step of monitoring the subject, e.g., for a change (e.g., an increase or decrease) in one or more of the diagnostic criteria for an SSD listed herein, or any other parameter related to clinical outcome. The subject can be monitored in one or more of the following periods: prior to beginning of treatment; during the treatment; or after one or more elements of the treatment have been administered. Monitoring can be used to evaluate the need for further treatment with the same or a different therapeutic agent or modality. Generally, a decrease in one or more of the parameters described above is indicative of the improved condition of the subject, although with red blood cell and platelet levels, an increase can be associated with the improved condition of the subject.

The methods can be used, for example, to choose between alternative treatments (e.g., a particular dosage, mode of delivery, time of delivery, inclusion of adjunctive therapy, e.g., administration in combination with a second agent) based on the subject's probable drug response. In some embodiments, a treatment for a subject having an SSD (e.g., SZ) is selected based on the subject's response allele, and the treatment is administered to the subject. In some embodiments, various treatments or combinations of treatments can be administered based on the presence in a subject of a response allele as described herein. Various treatment regimens are known for treating SSDs including, for example, regimens as described herein.

With regards to both prophylactic and therapeutic methods of treatment of SSDs, according to the present methods treatment can be specifically tailored or modified, based on knowledge obtained from pharmacogenomics. “Pharmacogenomics,” as used herein, refers to the application of genomics technologies such as structural chromosomal analysis, to drugs in clinical development and on the market. See, for example, Eichelbaum et al. (1996); Linder et al. (1997; Wang et al. (2003); Weinshilboum and Wang (2004); Guttmacher and Collins (2005); Weinshilboum and Wang (2006). Specifically, as used herein, the term refers the study of how a patient's genes determine his or her response to a drug (e.g., a patient's “drug response phenotype,” or “drug response allele”). Drug response phenotypes that are influenced by inheritance and can vary from potentially life-threatening adverse reactions at one of the spectrum to lack of therapeutic efficacy at the other. The ability to determine whether and how a subject will respond to a particular drug can assist medical professionals in determining whether the drug should be administered to the subject, and at what dose. Thus, this document provides methods for tailoring an individual's prophylactic or therapeutic treatment according to the presence of specific drug response alleles in that individual.

Standard pharmacologic therapies for SSDs include the administration of one or more antipsychotic medications, which are typically antagonists acting at postsynaptic D2 dopamine receptors in the brain. Antipsychotic medications include conventional, or first generation, antipsychotic agents, which are sometimes referred to as neuroleptics because of their neurologic side effects, and second generation antipsychotic agents, which are less likely to exhibit neuroleptic effects and have been termed atypical antipsychotics. Typical antipsychotics can include chlorpromazine, fluphenazine, haloperidol, thiothixene, trifluoperazine, perphenazine, and thioridazine; atypical antipsychotics can include aripiprazole, risperidone, clozapine, olanzapine, quetiapine, or ziprasidone.

Information generated from pharmacogenomic research using a method described herein can be used to determine appropriate dosage and treatment regimens for prophylactic or therapeutic treatment of an individual. This knowledge, when applied to dosing or drug selection, can avoid adverse reactions or therapeutic failure and thus enhance therapeutic or prophylactic efficiency when administering a therapeutic composition (e.g., a cytotoxic agent or combination of cytotoxic agents) to a patient as a means of treating or preventing progression of SSDs.

In some cases, a physician or clinician may consider applying knowledge obtained in relevant pharmacogenomics studies (e.g., using a method described herein) when determining whether to administer a pharmaceutical composition such as an antipsychotic agent or a combination of antipsychotic agents to a subject. In other cases, a physician or clinician may consider applying such knowledge when determining the dosage or frequency of treatments (e.g., administration of antipsychotic agent or combination of antipsychotic agents to a patient). As one example, a physician or clinician can determine (or have determined by, for example, a laboratory) the presence of one or more response alleles in a subject as described herein, and optionally one or more other markers associated with an SSD (e.g., SZ) or response to a treatment, of one or a group of subjects, e.g., clinical patients, or subjects who may be participating in a clinical trial, e.g., a trial designed to test the efficacy of a pharmaceutical composition (e.g., an antipsychotic or combination of antipsychotic agents); the physician or clinician can then correlate the genetic makeup of the subjects with their response to the pharmaceutical composition.

As another example, information regarding a response allele as described herein can be used to stratify or select a subject population for a clinical trial. The information can, in some embodiments, be used to stratify individuals that exhibit or are likely to exhibit a toxic response to a treatment from those that will not. In other cases, the information can be used to separate those that will be non-responders from those who will be responders. The alleles described herein can be used in pharmacogenomics-based design and manage the conduct of a clinical trial, e.g., as described in U.S. Pat. Pub. No. 2003/0108938.

As another example, information regarding a response allele as described herein, can be used to stratify or select human cells or cell lines for drug testing purposes. Human cells are useful for studying the effect of a polymorphism on physiological function, and for identifying and/or evaluating potential therapeutic agents for the treatment of SSDs, e.g., anti-psychotics. Thus the methods can include performing the present methods on genetic material from a cell line. The information can, in some embodiments, be used to separate cells that respond or are expected to respond to particular drugs from those that do not respond, e.g., which cells show altered second messenger signaling.

Also included herein are compositions and methods for the identification and treatment of subjects who have a predicted response to a treatment for an SSD (e.g., SZ), such that a theranostic approach can be taken to test such individuals to predict the effectiveness of a particular therapeutic intervention (e.g., a pharmaceutical or non-pharmaceutical intervention as described herein) and to alter the intervention to (1) reduce the risk of developing adverse outcomes and (2) enhance the effectiveness of the intervention. Thus, the methods and compositions described herein also provide a means of optimizing the treatment of a subject having such a disorder. Provided herein is a theranostic approach to treating and preventing SSDs, by integrating diagnostics and therapeutics to improve the real-time treatment of a subject. Practically, this means creating tests that can identify which patients are most suited to a particular therapy, and providing feedback on how well a drug is working to optimize treatment regimens.

Within the clinical trial setting, a theranostic method as described herein can provide key information to optimize trial design, monitor efficacy, and enhance drug safety. For instance, “trial design” theranostics can be used for patient stratification, determination of patient eligibility (inclusion/exclusion), creation of homogeneous treatment groups, and selection of patient samples that are representative of the general population. Such theranostic tests can therefore provide the means for patient efficacy enrichment, thereby minimizing the number of individuals needed for trial recruitment. “Efficacy” theranostics are useful for monitoring therapy and assessing efficacy criteria. Finally, “safety” theranostics can be used to prevent adverse drug reactions or avoid medication error.

The methods described herein can include retrospective analysis of clinical trial data as well, both at the subject level and for the entire trial, to detect correlations between an allele as described herein and any measurable or quantifiable parameter relating to the outcome of the treatment, e.g., efficacy (the results of which may be binary (i.e., yes and no) as well as along a continuum), side-effect profile (e.g., weight gain, metabolic dysfunction, lipid dysfunction, movement disorders, or extrapyramidal symptoms), treatment maintenance and discontinuation rates, return to work status, hospitalizations, suicidality, total healthcare cost, social functioning scales, response to non-pharmacological treatments, and/or dose response curves. The results of these correlations can then be used to influence decision-making, e.g., regarding treatment or therapeutic strategies, provision of services, and/or payment. For example, a correlation between a positive outcome parameter (e.g., high efficacy, low side effect profile, high treatment maintenance/low discontinuation rates, good return to work status, low hospitalizations, low suicidality, low total healthcare cost, high social function scale, favorable response to non-pharmacological treatments, and/or acceptable dose response curves) and a selected allele or genotype can influence treatment such that the treatment is recommended or selected for a subject having the selected allele or genotype.

This document also provides methods and materials to assist medical or research professionals in determining whether a particular treatment regimen is optimal. Medical professionals can be, for example, doctors, nurses, medical laboratory technologists, and pharmacists. Research professionals can be, for example, principle investigators, research technicians, postdoctoral trainees, and graduate students. A professional can be assisted by (1) determining whether specific polymorphic variants are present in a biological sample from a subject, and (2) communicating information about polymorphic variants to that professional.

Using information about specific polymorphic variants determined using a method described herein, a medical professional can take one or more actions that can affect patient care. For example, a medical professional can record information in the patient's medical record regarding the patient's likely response to a given treatment for an SSD (e.g., SZ). In some cases, a medical professional can record information regarding a treatment assessment, or otherwise transform the patient's medical record, to reflect the patient's current treatment and response allele(s). In some cases, a medical professional can review and evaluate a patient's entire medical record and assess multiple treatment strategies for clinical intervention of a patient's condition.

A medical professional can initiate or modify treatment after receiving information regarding a patient's response allele(s), for example. In some cases, a medical professional can recommend a change in therapy based on the subject's response allele(s). In some cases, a medical professional can enroll a patient in a clinical trial for, by way of example, detecting correlations between an allele or genotype as described herein and any measurable or quantifiable parameter relating to the outcome of the treatment as described above.

A medical professional can communicate information regarding a patient's expected response to a treatment to a patient or a patient's family. In some cases, a medical professional can provide a patient and/or a patient's family with information regarding SSDs and response assessment information, including treatment options, prognosis, and referrals to specialists. In some cases, a medical professional can provide a copy of a patient's medical records to a specialist.

A research professional can apply information regarding a subject's response allele(s) to advance scientific research. For example, a researcher can compile data on specific polymorphic variants. In some cases, a research professional can obtain a subject's response allele(s) as described herein to evaluate a subject's enrollment, or continued participation, in a research study or clinical trial. In some cases, a research professional can communicate information regarding a subject's response allele(s) to a medical professional. In some cases, a research professional can refer a subject to a medical professional.

Any appropriate method can be used to communicate information to another person (e.g., a professional). For example, information can be given directly or indirectly to a professional. For example, a laboratory technician can input a patient's polymorphic variant alleles as described herein into a computer-based record. In some cases, information is communicated by making a physical alteration to medical or research records. For example, a medical professional can make a permanent notation or flag a medical record for communicating the response allele determination to other medical professionals reviewing the record. In addition, any type of communication can be used to communicate allelic, genotypic, and/or treatment information. For example, mail, e-mail, telephone, and face-to-face interactions can be used. The information also can be communicated to a professional by making that information electronically available to the professional. For example, the information can be communicated to a professional by placing the information on a computer database such that the professional can access the information. In addition, the information can be communicated to a hospital, clinic, or research facility serving as an agent for the professional.

### V. ARTICLES OF MANUFACTURE

Also provided herein are articles of manufacture comprising a probe that hybridizes with a region of human chromosome as described herein and can be used to detect a polymorphism described herein. For example, any of the probes for detecting polymorphisms described herein can be combined with packaging material to generate articles of manufacture or kits. The kit can include one or more other elements including: instructions for use; and other reagents such as a label or an agent useful for attaching a label to the probe. Instructions for use can include instructions for diagnostic applications of the probe for predicting response to a treatment for SSDs in a method described herein. Other instructions can include instructions for attaching a label to the probe, instructions for performing in situ analysis with the probe, and/or instructions for obtaining a sample to be analyzed from a subject. In some cases, the kit can include a labeled probe that hybridizes to a region of human chromosome as described herein.

The kit can also include one or more additional reference or control probes that hybridize to the same chromosome or another chromosome or portion thereof that can have an abnormality associated with a particular response. A kit that includes additional probes can further include labels, e.g., one or more of the same or different labels for the probes. In other embodiments, the additional probe or probes provided with the kit can be a labeled probe or probes. When the kit further includes one or more additional probe or probes, the kit can further provide instructions for the use of the additional probe or probes. Kits for use in self-testing can also be provided. Such test kits can include devices and instructions that a subject can use to obtain a biological sample (e.g., buccal cells, blood) without the aid of a health care provider. For example, buccal cells can be obtained using a buccal swab or brush, or using mouthwash.

Kits as provided herein can also include a mailer (e.g., a postage paid envelope or mailing pack) that can be used to return the sample for analysis, e.g., to a laboratory. The kit can include one or more containers for the sample, or the sample can be in a standard blood collection vial. The kit can also include one or more of an informed consent form, a test requisition form, and instructions on how to use the kit in a method described herein. Methods for using such kits are also included herein. One or more of the forms (e.g., the test requisition form) and the container holding the sample can be coded, for example, with a bar code for identifying the subject who provided the sample.

### VI. DATABASES AND REPORTS

Also provided herein are databases that include a list of polymorphisms as described herein, and wherein the list is largely or entirely limited to polymorphisms identified as useful for predicting a subject's response to a treatment for an SSD (e.g., SZ) as described herein. The list is stored, e.g., on a flat file or computer-readable medium. The databases can further include information regarding one or more subjects, e.g., whether a subject is affected or unaffected, clinical information such as endophenotype, age of onset of symptoms, any treatments administered and outcomes (e.g., data relevant to pharmacogenomics, diagnostics or theranostics), and other details, e.g., about the disorder in the subject, or environmental or other genetic factors. The databases can be used to detect correlations between a particular allele or genotype and the information regarding the subject.

The methods described herein can also include the generation of reports, e.g., for use by a patient, care giver, payor, or researcher, that include information regarding a subject's response allele(s), and optionally further information such as treatments administered, treatment history, medical history, predicted response, and actual response. The reports can be recorded in a tangible medium, e.g., a computer-readable disk, a solid state memory device, or an optical storage device.

### VII. ENGINEERED CELLS

Also provided herein are engineered cells that harbor one or more polymorphisms described herein, e.g., one or more response alleles. Such cells are useful for studying the effect of a polymorphism on physiological function, and for identifying and/or evaluating potential therapeutic agents such as anti-psychotics for the treatment of an SSD (e.g., SZ).

As one example, included herein are cells in which one or more of the various alleles of the genes described herein has be re-created that is associated with a response to a specific treatment. Methods are known in the art for generating cells, e.g., by homologous recombination between the endogenous gene and an exogenous DNA molecule introduced into a cell, e.g., a cell of an animal. In some cases, the cells can be used to generate transgenic animals using methods known in the art.

The cells are preferably mammalian cells (e.g., neuronal type cells) in which an endogenous gene has been altered to include a polymorphism as described herein. Techniques such as targeted homologous recombinations, can be used to insert the heterologous DNA as described in, e.g., U.S. Pat. No. 5,272,071; WO 91/06667.

### VIII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Markers Associated with Antipsychotic Response

The Clinical Antipsychotic Trials of Intervention Effectiveness (CATIE), a large federally funded clinical trial designed to assess the efficacy of antipsychotics in a real world setting, is a valuable resource for determining the role of genes in drug response (Lieberman et al., 2005; Stroup et al., 2003).

The design of the CATIE study has been described in detail by others (Lieberman et al., 2005; Stroup et al., 2003). Briefly, 1460 subjects were randomly assigned one of several antipsychotics and those who did not respond or chose to quit their current medication were re-randomized to another drug.

As part of the CATIE trial, SNP genotyping was performed for roughly half of the trial participants (Sullivan et al., 2008). Treatment response and baseline phenotype data for the CATIE trial were made available to the inventors through the NIMH Center for Collaborative Genetic Studies on Mental Disorders (CCGMSD). Prior analysis of a sample comprising all 417 patients with schizophrenia and 419 unaffected controls self-reported as having exclusively European ancestry confirmed that this patient population contained no population stratification (Sullivan et al., 2008).

As described in detail below, the inventors used the genotyping results from CCGMSD combined with disease status, PANSS scores, and clinical drug response data, to design a custom genotyping platform that evaluated novel SNPs for possible utility in predicting responses to antipsychotic medications (Liu et al., 2012).

For the CATIE study, individual cases were diagnosed as having SZ based on DSM-III/IV criteria. Treatment response for all patients was assessed using the Positive and Negative Syndrome Scale (PANSS) (Kay et al., 1987; Kay et al., 1989; Leucht et al., 2005). PANSS rating was performed at baseline (after a minimum 7 day drug free period) and at various time points throughout the study. To avoid possible bias in post hoc selection of a treatment response variable, the inventors used the mixed model repeated measures (MMRM) approach developed by van den Oord and coworkers (van den Oord et al., 2009). Briefly, this model assumes 30-day delay for treatment effects, which was adjusted by baseline PANSS; it also models random effect by introducing random intercept allowing the intercepts to be different across subjects (Liu et al., 2012).

Selection of genes for novel analysis. An initial list of candidate loci was generated based on genetic association analyses using genotypes and phenotypes provided by the CCGSMD. Phenotypes for the CATIE study included baseline psychopathology and drug response variables described in detail by others (Lieberman et al., 2005; Stroup et al., 2003; Sullivan et al., 2008). Case control status and genotypes for the GAIN schizophrenia (version phs000021.v2.p1) and bipolar disorder (version phs000017.v3.p1) sample sets were obtained from the Database of Genotypes and Phenotypes (dbGaP), Bethesda (Md.): National Center for Biotechnology Information, National Library of Medicine (Manolio et al., 2007).

Initial screens using the CATIE sample involved genetic association of quantitative traits by linear regression using PLINK (version 1.04) (Purcell et al., 2007). The pharmacogenomic (PGx) phenotypes used for the screen were change in PANSS (percent relative to baseline) at last observation carried forward (Hamer et al., 2009) and time to “all cause” discontinuation, the primary endpoint of the CATIE Trial (Stroup et al., 2003), for each of the five antipsychotics included in the trial. Genes having one or more SNPs within the transcribed region with P values ≦10⁻² or associated intergenic SNPs with P values ≦10⁻³ were included in the initial PGx list. Similarly, screening was performed using association with baseline PANSS values. Genes having SNPs within the transcribed region associating with PANSS Total Score, or PANSS Positive, Negative or General subscale scores (P≦10⁻²) or with any of the 30 individual PANSS items (P≦10⁻³) were included.

Additional candidate loci were selected by case/control comparisons employing the GAIN consortium schizophrenia and bipolar disorder samples using an additive genetic model in PLINK. Genes with one or more SNPs within the transcribed region with P values ≦10⁻³ were included on the initial list.

To further focus the analysis, the final candidate gene list included only those loci with two or more SNPs meeting the above criteria. A total of about 2,700 genes passed this triage. Of these, approximately 700 contained blocks of linkage disequilibrium (in Caucasians) poorly covered by the original CCGSMD genotypes.

Selection of novel haplotype-tagging SNPs for the custom chip. To maximize coverage of the transcribed regions of the selected genes, the inventors identified blocks of linkage disequilibrium (LD) that were poorly represented by the genotypes provided by the CATIE consortium. Databases were constructed for SNPs mapping in the transcribed regions and within 5 kb in either direction using both CCGSMD-provided genotypes and genotypes downloaded from the international HapMap Project (on the world wide web at hapmap.ncbi.nlm.nih.gov/).

The Haploview program (Barrett et al., 2005; Barrett et al., 2009) (version 4.1) was used separately on each data set to define haplotype blocks and tagging SNPs (using an r² threshold of 0.8 to define tagging SNPs, and considering only haplotypes of frequency ≧0.01) and a database was generated that compared the resulting LD blocks for both samples in a contiguous manner based on the position of each SNP in the genome. Tagging SNPs from the HapMap Project, having minor allele frequencies of ≧0.01 and falling between the LD blocks in the CATIE sample, were selected for further analysis.

In addition, 2,060 SNPs with possible functional significance were included. Data on the functional class of SNPs (synonymous, non-synonymous, 3′ or 5′ UTRs) were downloaded from the NCBI database. The list of SNPs potentially affecting miRNA binding sites was obtained from PolymiRTS Database 2.0 (Bao et al., 2007; Ziebarth et al., 2012). SNPs with non-intronic functional annotations and with a minor allele frequency ≧0.01, based on NCBI resources, were selected. The list of putative functional SNPs was compared with the list of SNPs used to tag LD blocks, and redundancies were removed.

So as to ensure that newly analyzed SNPs would provide the richest possible source of genetic information, as a final triage the inventors excluded most SNPs that could be imputed with high probability using the genotype data provided by the CATIE consortium. Briefly, the approximately 450,000 genotypes already available were used to impute SNP genotypes on a genome-wide basis with the BEAGLE program (version 3.0.4; Browning et al., 2009) using the HapMap Caucasian (CEU) trios as reference. This produced an output of imputed genotypes along with an assigned probability for each imputed genotype. The inventors next created a database that contained only SNPs with a mean imputed probability ≧0.8 across all of the CATIE samples and used this as an exclusion list for SNP selection.

Design of Infinium HD iSelect Custom BeadChip.

The above process identified approximately 10,000 SNPs. In addition, for quality control (QC) and confirmatory purposes, the inventors included 281 SNPs previously genotyped by the CATIE group and approximately 500 SNPs previously evaluated by the inventors in non-CATIE schizophrenia or bipolar patients. Finally, to test the feasibility of using Illumina's iSelect BeadChip platform to detect for copy number variant (CNV) regions, the inventors included 200 SNPs that they had identified as CNV in the GAIN sample using the Affymetrix Genome-Wide Human SNP Array 6.0 platform.

The inventors designed a 10,000 bead, iSelect BeadChip obtained from Illumina Inc. (San Diego, Calif.). The assay design requirements (approximately 30% of SNPs require 2 beads rather than 1) required a further reduction in the number of SNPs. To accommodate this, approximately 3,500 SNPs were eliminated due to the fact that they were included solely to capture LD blocks in large genes that displayed genetic association with only two of the many analyzed phenotypes. Of the ˜8,500 SNPs remaining, about 9% could not be accommodated by the iSelect platform as determined by Illumina's bioinformatics analysis.

In total, 7,584 SNPs located in or near 1,711 genes were included on the BeadChip. Of this total, 7,303 SNPS have not been previously analyzed for the CATIE sample. The majority of these (4,719 in or near 638 genes) covered gaps between LD blocks in candidate loci. The remaining 2,584 SNPs (in 1,445 genes) have putative functional significance or prior evidence in other sample sets suggesting a role in schizophrenia or bipolar disorder.

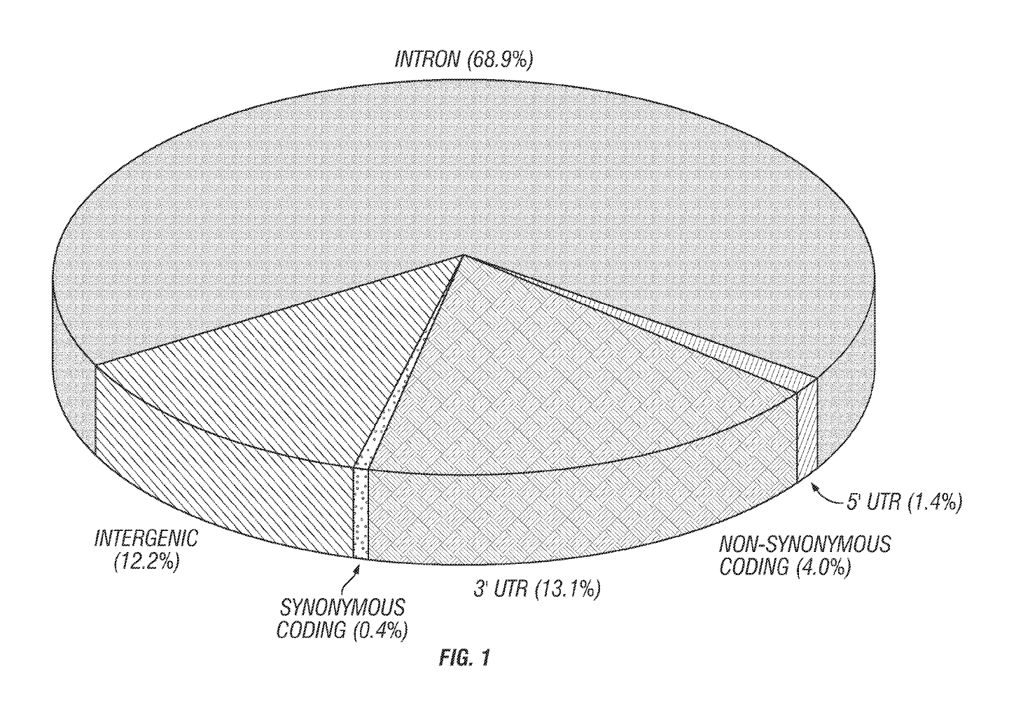

FIG. 1 summarizes the functional classification for the SNPs included on the custom iSelect BeadChip. Most of the SNPs are intronic (68.9%) and were included to cover LD blocks not well represented in the CATIE-provided genotypes. Additionally, the BeadChip had relatively high representations of SNPs categorized as intergenic (12.2%), 3′ UTR (13.1%), and non-synonymous coding (4.0%). The smallest functional categories were 5′ UTR (1.4%) and synonymous coding (0.4%) variants.

Methods for Identification of Novel Haplotype-Tagging SNPs Prediction Response to Antipsychotic Medications.

The design of the CATIE study, including details of consent for genetic analyses, has been described in detail by others (Lieberman et al., 2005; Stroup et al., 2003; Sullivan et al., 2008). Only retrospective genetic analyses, judged to be exempt from human studies requirements by an IRB, were conducted in the current study. Consented DNA samples were obtained from the Rutgers University Cell and DNA Repository in collaboration with CCGSMD. The inventors genotyped a total of 407 DNA samples from Caucasian patients who participated in the CATIE study, distributed as follows in Phase I of the trial: olanzapine, 93; perphenazine, 76; quetiapine, 94; risperidone, 97; ziprasidone, 47. All of these patients self-reported as having exclusively European ancestry. This same patient population was described in detail in a previous study that confirmed that there is no hidden population stratification in the sample (Sullivan et al., 2008). The inventors genotyped an additional 429 samples (215 schizophrenia and 214 bipolar patients) from the GAIN consortium for QC purposes to allow comparisons to the previous genotypes obtained from dbGaP for these samples using the Affymetrix Genome-Wide Human SNP Array 6.0 (on the world wide web at ncbi.nlm.nih.gov/sites/entrez?db=gap).

Genotyping was performed on a fee for service basis according to Illumina's standard operating procedures. Raw intensity files were processed using Illumina® BeadStudio version 1.7.4 software. At the suggested general call threshold of 0.4, a total of 267 SNPs failed initial QC and were not analyzed further. Only two of these had been previously genotyped in the CATIE sample. For the 7,317 SNPs that passed this initial QC, the genotyping success rate across all samples was 98.9% (median 99.4%). Eighteen of these SNPs had success rates <80% and were not used for subsequent genetic analyses. On an individual sample basis, median genotyping success rates for the remaining SNPs across all SNPs averaged 96.1% (median 96.2%).

To allow comparison to previously published PGx findings for CATIE and to avoid possible bias in post-hoc selection of a treatment response variable, the inventors used the mixed model repeated measures (MMRM) approach developed by Van den Oord and coworkers (van den Oord et al., 2009; McClay et al., 2011). Briefly, this method models random effects by introducing random slopes for treatment effects, allowing treatment effects to be different across subjects. The MMRM approach serves to increase the statistical power to detect genetic associations by increasing the precision in measuring change in PANSS Total Score (PANSS-T) by accounting for variance due to baseline PANSS-T, and treatment, as well as smoothing out the random fluctuations in PANSS-T between visits due to various uncontrolled variables.

Change in PANSS-T was modeled for Phases 1, 1b, and 2 of the CATIE Study using a model that assumed a 30 day lag period with a constant drug effect after that point (van den Oord et al., 2009). Sample sizes for each of the drugs were as follows: olanzapine, 134; perphenazine, 75; quetiapine, 124; risperidone, 134; ziprasidone, 74. With a type 1 error rate of 0.05, a sample size of 124 gives 80% power for a SNP that explains 6% of the variance in the regression model, and a sample size of 71 gives 80% power for a SNP that explains 10% of the variance in the regression model. Though genotyping results were obtained for 7,303 SNPs not previously evaluated for CATIE, genetic association analysis was limited to 6,789 of these SNPs passing QC and having minor allele frequencies ≧3% in the combined sample of 836 CATIE and GAIN Caucasian patients. For these, the inventors tested the null hypothesis that there was no difference in mean PANSS-T change for patients carrying the minor allele of the SNP for the particular antipsychotic drug (additive model). The change in PANSS-T score was used as a continuous dependent variable using the SVS version 7.3.1 software package (Golden Helix Inc. Bozeman, Mont.). Quantile-Quantile (QQ) plots were prepared using the R statistical package version 2.14.1. For comparison purposes, original CATIE-provided SNP genotypes in specific genes were evaluated using the same genetic analysis. Haplotype association integrating newly generated and original genotypes for specific regions was carried out in SVS using haplotype blocks predefined by Haploview software.

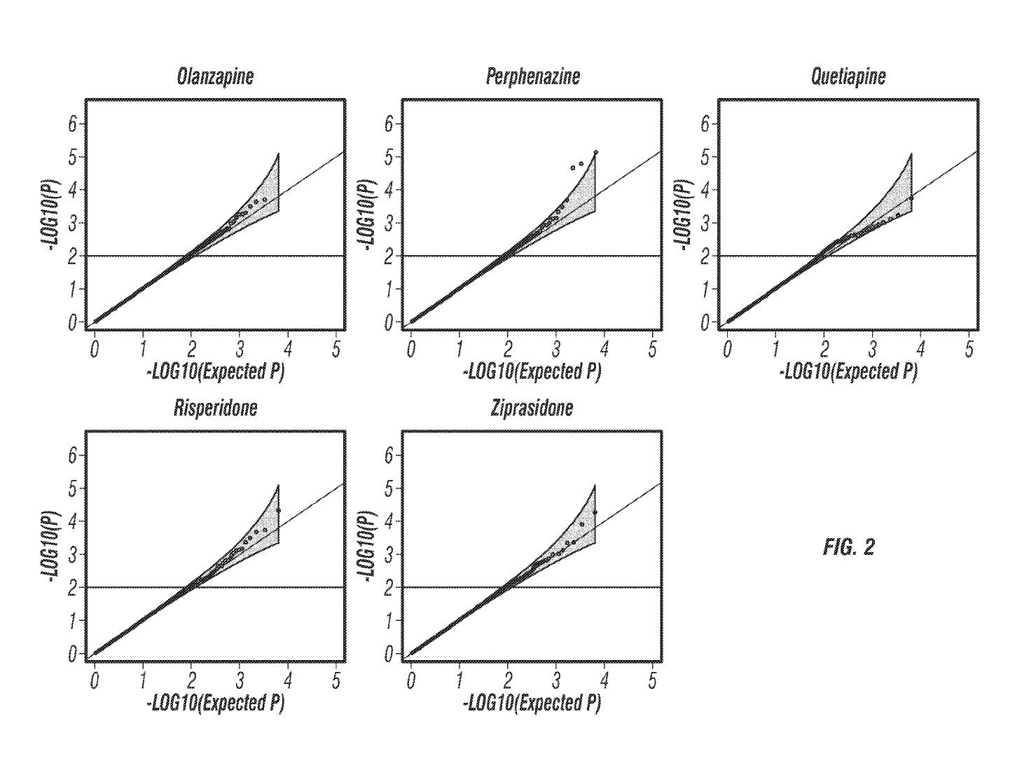

FIG. 2 shows the QQ plots for each of the five drugs for the 6,789 SNPs not previously evaluated in CATIE (MAF≧3% within the individual drug arm). These results indicate that the custom BeadChip design resulted in a modest enrichment for SNPs influencing response to four of the five antipsychotics, with quetiapine being the sole exception.

The newly generated genotypes were integrated with those provided by the CATIE consortium followed by association analysis with the identical patient samples. This analysis confirmed that most of the SNPs tag novel haplotypes or genetically isolated regions that could not have been detected or imputed using the original CATIE genotypes. For example, twelve of the 20 most significant SNPs define novel haplotypes, and 11 of these 12 are sufficient to tag the particular haplotype.

### Example 2

### Novel Haplotype-Tagging SNPs Impacting Response for Olanzapine

Table 1A provides numerous examples of SNP alleles that predict good response to olanzapine, and table 1B provides numerous examples of SNP alleles that predict poor response to olanzapine. Tables 1A and 1B report the SNPs, SNP-alleles, P values, and Beta weights (in PANSS-Total units) from the linear regression for SNPs that affect response to olanzapine. A negative beta weight indicates that the allele is associated with a decrease in PANSS-T score, corresponding to greater improvement (or lowering) of symptom burden. A positive beta weight indicates that the allele is associated with an increase in PANSS-T score, corresponding to a worsening (or increase) of symptom burden. For each of the SNPs listed the reference SNP (rs) number is provided, which provides the known sequence context for the given SNP (see, e.g., National Center for Biotechnology Information (NCBI) SNP database available on the world wide web at ncbi.nlm.nih.gov/snp).

TABLE 1A
Alleles Influencing Good Response to Olanzapine

| Gene | NCBI RS# | Allele | Beta (PANSS) | P |
|---|---|---|---|---|
| CSMD1 | 17070785 | A | −9.11 | 1.66E−04 |
| PTPRN2 | 221253 | C | −13.90 | 3.33E−04 |
| KIAA0182 | 12149202 | A | −5.74 | 5.40E−04 |
| FREM1 | 16932300 | C | −7.73 | 5.83E−04 |
| CNTNAP2 | 7792198 | A | −5.80 | 5.87E−04 |
| PCDH15 | 10825137 | A | −4.89 | 9.66E−04 |
| PDGFD; DDI1 | 2279789 | C | −4.42 | 1.51E−03 |
| ROBO2 | 4343667 | C | −4.96 | 1.64E−03 |
| CNTN4 | 4685501 | T | −4.63 | 1.89E−03 |
| NAB1 | 2293765 | A | −4.60 | 1.99E−03 |
| CNTN4 | 1554561 | T | −4.50 | 1.99E−03 |
| GPC6 | 9301876 | A | −4.68 | 2.44E−03 |
| SCN3A | 10048748 | C | −5.44 | 2.89E−03 |
| RBFOX1 | 7194775 | C | −4.37 | 3.95E−03 |
| CA10 | 741682 | A | −5.58 | 4.08E−03 |
| CSMD1 | 2724972 | G | −4.69 | 4.38E−03 |
| SLC16A9 | 16913940 | A | −6.05 | 4.80E−03 |
| CSMD1 | 7815861 | C | −5.06 | 5.09E−03 |
| PKNOX2 | 1044314 | G | −4.14 | 5.48E−03 |
| PSD3 | 7016207 | C | −6.20 | 5.65E−03 |
| CLIC5 | 35822882 | G | −14.90 | 5.87E−03 |
| NELL1 | 4244549 | C | −4.96 | 5.96E−03 |
| KITLG | 995029 | C | −7.22 | 6.13E−03 |
| FBN3 | 2287937 | C | −5.34 | 6.29E−03 |
| LIMCH1 | 7659262 | A | −5.30 | 6.97E−03 |
| DENND5B | 11615961 | A | −3.73 | 7.70E−03 |
| HPS4 | 16982178 | A | −5.47 | 8.26E−03 |
| ERBB4 | 7558615 | C | −7.96 | 8.29E−03 |
| WNT5B | 2240513 | C | −3.80 | 8.68E−03 |
| ANO2 | 7959306 | A | −4.98 | 8.81E−03 |
| ADTRP | 6929400 | C | −3.98 | 8.91E−03 |
| PPARG | 9809905 | G | −3.74 | 9.59E−03 |
| LOC100129345 | 12435621 | G | −6.10 | 9.63E−03 |
| JPH2 | 1055689 | C | −4.34 | 9.80E−03 |
| CHN2 | 10486608 | C | −5.68 | 1.01E−02 |
| CSMD1 | 2724973 | A | −5.11 | 1.01E−02 |
| CDH23 | 10823827 | G | −4.02 | 1.03E−02 |
| ANK3 | 4611159 | A | −6.48 | 1.06E−02 |
| NOS1AP | 1504424 | C | −9.99 | 1.07E−02 |
| GALNTL4 | 923259 | A | −4.61 | 1.14E−02 |
| NAB1 | 4853724 | C | −4.61 | 1.16E−02 |
| VDAC1 | 6880980 | G | −7.14 | 1.17E−02 |
| ELOVL7 | 13358053 | C | −5.16 | 1.24E−02 |
| CNTNAP2 | 12670106 | A | −3.60 | 1.24E−02 |
| DLG2 | 2512676 | G | −4.08 | 1.25E−02 |
| CACNA2D3 | 1568982 | C | −5.10 | 1.27E−02 |
| LYN | 7840325 | A | −3.83 | 1.27E−02 |
| CDH20 | 1539996 | C | −4.47 | 1.28E−02 |
| CGNL1 | 12913924 | A | −4.44 | 1.29E−02 |
| CNTNAP2 | 9640235 | A | −3.70 | 1.30E−02 |
| CPNE4 | 1381078 | C | −5.23 | 1.33E−02 |
| NTNG2 | 1810887 | A | −3.65 | 1.33E−02 |
| SCD5 | 10516679 | C | −4.54 | 1.40E−02 |
| ZNF804A | 2369593 | A | −6.03 | 1.41E−02 |
| OPCML | 12417211 | C | −4.66 | 1.45E−02 |
| GRIN3A | 2485528 | C | −4.11 | 1.48E−02 |
| WDR48 | 1053516 | A | −3.88 | 1.53E−02 |
| HPCAL1 | 887981 | C | −4.41 | 1.56E−02 |
| IFT74 | 10511795 | C | −6.93 | 1.66E−02 |
| IFT74 | 10511797 | C | −6.93 | 1.66E−02 |
| FMN2 | 7543271 | A | −3.63 | 1.67E−02 |
| FBN3 | 35999680 | A | −11.00 | 1.70E−02 |
| MAGI2 | 6952164 | A | −3.96 | 1.78E−02 |
| BRE; | 13031756 | A | −3.42 | 1.81E−02 |
| LOC100505716 | | | | |
| GRIA1 | 514381 | C | −4.02 | 1.84E−02 |
| KIAA1797 | 7030093 | C | −3.95 | 1.95E−02 |
| FAM46C | 866111 | A | −5.43 | 2.02E−02 |
| TRPM3 | 3010423 | A | −3.88 | 2.06E−02 |
| GRB10 | 2329486 | A | −4.28 | 2.07E−02 |
| SLIT1 | 4917756 | A | −4.75 | 2.13E−02 |
| CACNA2D3 | 4642090 | C | −5.21 | 2.16E−02 |
| PHACTR3 | 6026976 | A | −6.55 | 2.17E−02 |
| NCAM2 | 2826730 | C | −4.63 | 2.18E−02 |
| FKTN | 34787999 | A | −3.75 | 2.18E−02 |
| CTBP2 | 11599580 | C | −12.50 | 2.19E−02 |
| XPR1 | 1061015 | C | −3.61 | 2.22E−02 |
| SRRM4 | 4767785 | C | −3.51 | 2.30E−02 |
| LOC100130887 | 10887024 | A | −3.49 | 2.30E−02 |
| RGS6 | 11624306 | A | −4.26 | 2.31E−02 |
| SEPT9 | 34587622 | C | −5.29 | 2.37E−02 |
| CNTNAP2 | 2249958 | C | −4.95 | 2.37E−02 |
| ANK3 | 12354956 | A | −4.59 | 2.51E−02 |
| CYP4V2 | 1053094 | T | −3.41 | 2.56E−02 |
| ERBB4 | 6712295 | A | −3.95 | 2.61E−02 |
| SAMD12 | 2514591 | A | −4.58 | 2.65E−02 |
| CSMD1 | 2616996 | G | −3.79 | 2.67E−02 |
| ZNF169 | 9409513 | C | −3.28 | 2.69E−02 |
| SGCZ | 1454583 | C | −4.20 | 2.71E−02 |
| XPR1 | 1061016 | C | −3.44 | 2.72E−02 |
| SGCZ | 1454580 | C | −3.57 | 2.73E−02 |
| DENND5B | 708200 | G | −3.11 | 2.81E−02 |
| FMNL2 | 12612608 | A | −3.44 | 2.82E−02 |
| CGNL1 | 1908202 | C | −3.79 | 2.83E−02 |
| GPC6 | 1538195 | G | −3.40 | 2.86E−02 |
| CAMK2D | 13113625 | C | −3.68 | 2.87E−02 |
| APCDD1 | 1045584 | G | −3.41 | 2.88E−02 |
| RORA | 12910376 | C | −3.38 | 2.90E−02 |
| MACROD2 | 461651 | A | −3.59 | 2.92E−02 |
| ITPR1 | 11717244 | C | −3.53 | 2.94E−02 |
| GAS7 | 9904524 | G | −4.96 | 2.95E−02 |
| MEPE | 3749575 | A | −8.95 | 2.97E−02 |
| PARD3B | 17283257 | A | −3.82 | 2.97E−02 |
| RIMS1 | 9446639 | C | −3.56 | 2.97E−02 |
| BIK | 5996274 | A | −4.94 | 3.09E−02 |
| PCP4L1 | 6671288 | C | −4.15 | 3.17E−02 |
| PI4KA; | 78656 | C | −7.22 | 3.22E−02 |
| SERPIND1 | | | | |
| MTIF3 | 12585587 | A | −4.37 | 3.26E−02 |
| SLC35F3 | 12759054 | C | −3.34 | 3.27E−02 |
| FMNL2 | 4664113 | C | −4.25 | 3.30E−02 |
| DLGAP1 | 561434 | A | −4.43 | 3.32E−02 |
| FHIT | 971866 | A | −5.07 | 3.33E−02 |
| CDH13 | 3743621 | C | −5.54 | 3.35E−02 |
| FAM173B | 12652786 | C | −3.39 | 3.35E−02 |
| RASGEF1C | 2278661 | C | −3.04 | 3.37E−02 |
| MAGI2 | 848813 | C | −4.34 | 3.39E−02 |
| GRM8 | 17865066 | A | −7.97 | 3.40E−02 |
| GRM8 | 17865434 | C | −7.97 | 3.40E−02 |
| CACNA1B | 12352971 | G | −4.21 | 3.40E−02 |
| SEC16B | 16852158 | C | −6.45 | 3.47E−02 |
| PCDH17 | 7319102 | A | −3.47 | 3.49E−02 |
| IL15 | 3806798 | A | −4.33 | 3.50E−02 |
| KCND2 | 10953911 | G | −5.26 | 3.53E−02 |
| KCND2 | 718805 | A | −5.26 | 3.53E−02 |
| MCPH1 | 930557 | C | −3.78 | 3.57E−02 |
| HTR1B | 11568817 | G | −3.16 | 3.59E−02 |
| SLC6A5 | 1443547 | A | −3.31 | 3.68E−02 |
| ARHGAP31 | 751607 | A | −4.30 | 3.69E−02 |
| LRP1B | 4591293 | A | −3.04 | 3.74E−02 |
| EML1 | 34198557 | C | −4.01 | 3.77E−02 |
| SGCZ | 10503525 | C | −3.05 | 3.79E−02 |
| PTPRT | 6030084 | A | −2.96 | 3.79E−02 |
| SPOCK1 | 1051854 | G | −6.02 | 3.80E−02 |
| KCNH1 | 4282878 | C | −3.08 | 3.81E−02 |
| FSTL5 | 13127909 | C | −4.60 | 3.83E−02 |
| NTRK2 | 4142909 | C | −3.81 | 3.83E−02 |
| NRXN1 | 11885824 | A | −5.21 | 3.89E−02 |
| KLHL29 | 17045819 | C | −4.81 | 3.93E−02 |
| KDM4C | 35389625 | C | −11.20 | 3.97E−02 |
| ERBB4 | 7565257 | A | −3.44 | 4.03E−02 |
| GAN | 1345895 | A | −3.53 | 4.07E−02 |
| RNF144A | 7605141 | C | −4.00 | 4.13E−02 |
| ATP10A | 2291351 | A | −4.99 | 4.15E−02 |
| ANK3 | 2893823 | A | −4.47 | 4.15E−02 |
| SLC16A4 | 12126959 | A | −4.16 | 4.17E−02 |
| NTSR2 | 7602721 | C | −4.08 | 4.20E−02 |
| WDR90 | 1057835 | A | −3.10 | 4.24E−02 |
| FAM170A | 2162856 | A | −3.06 | 4.25E−02 |
| AGAP1 | 4663220 | T | −3.07 | 4.27E−02 |
| PKP4 | 11680160 | A | −4.33 | 4.28E−02 |
| JPH2 | 2064381 | A | −3.06 | 4.28E−02 |
| MACROD2 | 6079429 | A | −4.72 | 4.29E−02 |
| ARVCF | 2238794 | C | −3.11 | 4.32E−02 |
| KAZN | 12130605 | C | −3.66 | 4.33E−02 |
| EXOC2 | 9405889 | C | −3.21 | 4.34E−02 |
| ANK3 | 10821660 | G | −4.45 | 4.41E−02 |
| CACNG4 | 7219571 | A | −5.84 | 4.42E−02 |
| IL1RAP | 2361835 | A | −3.93 | 4.42E−02 |
| IL1RAP | 7642607 | A | −3.93 | 4.42E−02 |
| IL1RAP | 7642797 | A | −3.93 | 4.42E−02 |
| NTRK2 | 1490403 | A | −3.10 | 4.51E−02 |
| LDB2 | 872478 | C | −3.53 | 4.62E−02 |
| FGF14 | 2390674 | A | −4.22 | 4.67E−02 |
| PRDM2 | 6429793 | A | −3.13 | 4.73E−02 |
| ZNF169 | 9409514 | C | −3.04 | 4.77E−02 |
| MAGI2 | 12705833 | G | −2.87 | 4.79E−02 |
| ERG | 9981408 | G | −3.15 | 4.80E−02 |
| SGCZ | 13278000 | A | −2.91 | 4.83E−02 |
| EHD4 | 1048166 | C | −3.04 | 4.85E−02 |
| EHD4 | 1048175 | C | −3.04 | 4.85E−02 |
| CPNE5 | 12194367 | T | −3.14 | 4.92E−02 |
| PEBP4 | 13271643 | C | −5.41 | 4.95E−02 |
| KCND2 | 11765060 | C | −5.30 | 4.95E−02 |
| NTRK2 | 1490407 | G | −3.00 | 4.96E−02 |
| IFT74 | 4463511 | C | −3.77 | 4.99E−02 |
| DLGAP1 | 498419 | A | −3.07 | 4.99E−02 |
| ADAMTS9-AS2 | 17071651 | C | −3.03 | 4.99E−02 |

TABLE 1B
Alleles Influencing Poor Response to Olanzapine

| | | | Beta | |
|---|---|---|---|---|
| Gene | NCBI RS# | Allele | (PANSS) | P |
| PLAGL1; LOC100652728 | 2247408 | C | 5.63 | 2.07E−04 |
| PLAGL1; LOC100652728 | 3819811 | A | 5.72 | 2.43E−04 |
| ZNF71 | 11881987 | A | 5.24 | 7.21E−04 |
| DGKD | 838718 | A | 4.46 | 1.03E−03 |
| SEMA5A | 707637 | A | 5.81 | 1.54E−03 |
| GRIN3A | 4324970 | C | 4.89 | 2.15E−03 |
| DLG2 | 11234192 | G | 7.12 | 2.54E−03 |
| DLG2 | 555867 | A | 5.41 | 2.64E−03 |
| PARK2 | 7769196 | A | 4.70 | 2.82E−03 |
| DLG2 | 1943687 | G | 5.36 | 2.84E−03 |
| SEMA5A | 1457768 | A | 6.95 | 3.05E−03 |
| IL17RD | 9311641 | C | 7.27 | 3.26E−03 |
| TPH2 | 1872824 | C | 4.41 | 3.43E−03 |
| RAP1GAP2 | 10805 | G | 3.98 | 3.51E−03 |
| GRIK3 | 12059346 | C | 8.94 | 3.70E−03 |
| SEMA3F | 1046956 | A | 4.65 | 3.90E−03 |
| PCDH15 | 2384470 | C | 4.71 | 4.00E−03 |
| NPAS3 | 12887270 | A | 4.39 | 4.21E−03 |
| MAGI2 | 798338 | A | 4.21 | 4.42E−03 |
| AK5 | 12565526 | A | 4.31 | 4.47E−03 |
| FAM19A1 | 13094092 | C | 4.61 | 4.96E−03 |
| PPA2 | 923587 | C | 3.86 | 5.78E−03 |
| PTGER3 | 5682 | C | 3.94 | 5.87E−03 |
| CNTNAP2 | 802022 | C | 4.37 | 5.89E−03 |
| DLG2 | 7122815 | A | 5.32 | 6.00E−03 |
| CNTNAP2 | 2710157 | C | 4.14 | 7.13E−03 |
| DLGAP2 | 6992443 | A | 7.39 | 7.82E−03 |
| UNC13C | 10152907 | A | 4.17 | 7.95E−03 |
| COL22A1 | 7819082 | C | 6.88 | 8.17E−03 |
| SYN3 | 17772478 | A | 14.30 | 8.29E−03 |
| PCLO | 12666717 | A | 4.00 | 9.02E−03 |
| PCDH15 | 16937849 | C | 5.08 | 9.43E−03 |
| CNTNAP2 | 1608958 | C | 4.24 | 9.50E−03 |
| CNTNAP2 | 10488348 | C | 4.34 | 9.70E−03 |
| CNTNAP2 | 12670862 | C | 4.44 | 9.95E−03 |
| PLD1 | 416158 | A | 5.42 | 1.02E−02 |
| ANK3 | 10740023 | C | 4.34 | 1.12E−02 |
| ANK3 | 2061486 | A | 4.34 | 1.12E−02 |
| NALCN | 8000980 | C | 6.10 | 1.16E−02 |
| INS-IGF2; IGF2 | 3213221 | C | 3.82 | 1.27E−02 |
| MTIF3 | 17085633 | C | 3.95 | 1.27E−02 |
| TSNAX-DISC1 | 1411776 | A | 4.78 | 1.30E−02 |
| DGKD | 838717 | A | 3.57 | 1.32E−02 |
| TMC8 | 454138 | C | 3.66 | 1.42E−02 |
| CSMD1 | 595834 | C | 4.55 | 1.42E−02 |
| KCNJ2 | 9914095 | A | 5.26 | 1.44E−02 |
| DLG2 | 4505088 | G | 5.13 | 1.45E−02 |
| SLC1A3 | 891189 | A | 3.70 | 1.50E−02 |
| KLHL32 | 13203153 | A | 4.22 | 1.57E−02 |
| WWC1 | 10042345 | C | 3.55 | 1.59E−02 |
| KCNN2 | 4466150 | C | 4.33 | 1.61E−02 |
| ARVCF | 1990276 | C | 3.93 | 1.68E−02 |
| KCNQ1 | 800336 | A | 4.07 | 1.71E−02 |
| CNTN4 | 1178491 | A | 3.53 | 1.83E−02 |
| CSMD1 | 4875769 | A | 3.68 | 1.85E−02 |
| NALCN | 17622020 | A | 4.15 | 1.86E−02 |
| CSMD3 | 1895013 | A | 6.99 | 1.86E−02 |
| CNTN4 | 2727902 | G | 3.43 | 1.88E−02 |
| GPSM1 | 7853207 | C | 3.92 | 1.88E−02 |
| SLC25A18 | 1044497 | C | 4.56 | 1.90E−02 |
| CTNNA3 | 1941996 | A | 4.19 | 1.97E−02 |
| ITPR1 | 4685786 | A | 4.76 | 1.98E−02 |
| CDH13 | 16958456 | A | 4.39 | 2.02E−02 |
| SYNPR | 11920956 | G | 3.33 | 2.05E−02 |
| IGF2R | 8191754 | C | 5.13 | 2.06E−02 |
| PRICKLE2 | 704378 | C | 3.56 | 2.08E−02 |
| SLC35F3 | 4564211 | G | 5.81 | 2.12E−02 |
| C9orf84 | 7869279 | C | 3.61 | 2.14E−02 |
| PLCB1 | 6086410 | C | 3.80 | 2.14E−02 |
| DOK6 | 2034022 | A | 3.18 | 2.19E−02 |
| CDH13 | 11150543 | A | 3.84 | 2.20E−02 |
| GFRA1 | 2694766 | A | 3.53 | 2.23E−02 |
| ATXN3 | 2896196 | C | 4.67 | 2.30E−02 |
| ERC2 | 2047566 | C | 3.62 | 2.32E−02 |
| FAM186A | 6580742 | C | 4.84 | 2.34E−02 |
| PACRG | 12526892 | C | 3.72 | 2.35E−02 |
| KCNIP1 | 10050842 | A | 3.26 | 2.36E−02 |
| CNTNAP2 | 10263151 | C | 3.55 | 2.36E−02 |
| PARD3B | 10209145 | C | 3.22 | 2.37E−02 |
| IGF1R | 867431 | A | 3.54 | 2.38E−02 |
| ITGA1 | 1047483 | A | 3.59 | 2.39E−02 |
| ITGA1 | 6895049 | G | 3.59 | 2.39E−02 |
| ASAP1 | 7387355 | A | 4.03 | 2.39E−02 |
| ARVCF | 917479 | G | 3.43 | 2.42E−02 |
| F13A1 | 5987 | A | 4.35 | 2.44E−02 |
| PCDH15 | 12772330 | A | 4.54 | 2.53E−02 |
| DLG2 | 1943691 | A | 3.34 | 2.54E−02 |
| ALS2 | 7572898 | C | 4.17 | 2.58E−02 |
| LOC100505973 | 1537080 | A | 3.73 | 2.64E−02 |
| ASPHD2 | 34902186 | A | 6.30 | 2.81E−02 |
| CNTNAP2 | 6957194 | A | 3.62 | 2.82E−02 |
| SLC25A21 | 848097 | C | 3.49 | 2.83E−02 |
| ARVCF | 2240717 | C | 3.44 | 2.88E−02 |
| MICAL2 | 10765924 | A | 3.75 | 2.88E−02 |
| PRUNE2 | 1425286 | C | 5.79 | 2.88E−02 |
| SAMD4A | 1211170 | A | 3.19 | 2.90E−02 |
| MBP | 12456475 | C | 3.74 | 2.90E−02 |
| NALCN | 17622124 | C | 4.06 | 2.92E−02 |
| CSMD1 | 6558759 | C | 4.48 | 2.92E−02 |
| TRPC4 | 1570608 | G | 3.19 | 2.96E−02 |
| RARB | 4393871 | C | 3.36 | 2.99E−02 |
| SORCS3 | 2496017 | A | 4.21 | 3.01E−02 |
| LRP1B | 736602 | A | 3.73 | 3.02E−02 |
| TMC5 | 16972065 | C | 6.03 | 3.05E−02 |
| RBFOX2 | 6000085 | C | 7.29 | 3.07E−02 |
| LOC100505501 | 7837298 | C | 3.52 | 3.08E−02 |
| CDH13 | 12596958 | C | 3.52 | 3.10E−02 |
| BLZF1 | 2275300 | G | 7.78 | 3.12E−02 |
| NEDD4L | 474743 | C | 3.02 | 3.13E−02 |
| CSMD1 | 6988561 | G | 3.42 | 3.14E−02 |
| DAOA-AS1 | 12584489 | A | 6.56 | 3.15E−02 |
| ATP2B2 | 34903 | A | 3.12 | 3.22E−02 |
| DLG2 | 10792782 | A | 3.19 | 3.33E−02 |
| PPP1R9A | 10485996 | A | 4.41 | 3.34E−02 |
| KCNIP1 | 50057 | A | 3.80 | 3.36E−02 |
| SEMA5A | 1505067 | C | 3.12 | 3.40E−02 |
| DLG2 | 7103862 | C | 3.15 | 3.40E−02 |
| KLHL29 | 4665614 | C | 3.41 | 3.47E−02 |
| DLG2 | 553071 | A | 3.44 | 3.59E−02 |
| CTNNA2 | 408144 | A | 3.02 | 3.60E−02 |
| ERCC6 | 2228527 | A | 3.86 | 3.64E−02 |
| PARK2 | 10945755 | C | 3.71 | 3.68E−02 |
| ZNF638 | 2257136 | G | 5.82 | 3.69E−02 |
| ANGPT1 | 1954727 | C | 3.32 | 3.72E−02 |
| CERK | 801720 | G | 3.33 | 3.77E−02 |
| PTPRN2 | 4716835 | A | 4.80 | 3.78E−02 |
| GPM6A; LOC100506176 | 13136033 | A | 3.37 | 3.81E−02 |
| QRFPR | 11737010 | A | 3.32 | 3.82E−02 |
| SDK1 | 659182 | C | 3.60 | 3.83E−02 |
| ASAP1 | 11992957 | C | 3.15 | 3.84E−02 |
| CNTNAP2 | 11984392 | C | 3.29 | 3.84E−02 |
| GPSM1 | 3812550 | A | 3.35 | 3.86E−02 |
| CSMD1 | 667595 | C | 4.33 | 3.86E−02 |
| C8orf34 | 2591003 | A | 3.76 | 3.87E−02 |
| CNTNAP2 | 10240221 | A | 3.17 | 3.89E−02 |
| RNF144A | 309304 | A | 3.06 | 3.93E−02 |
| NAV3 | 770108 | C | 3.40 | 3.93E−02 |
| PCLO | 2057899 | C | 3.10 | 3.96E−02 |
| ERBB4 | 10200506 | A | 3.80 | 3.97E−02 |
| CNTNAP2 | 17585288 | C | 4.39 | 3.97E−02 |
| FHIT | 1350636 | A | 3.20 | 4.04E−02 |
| SULT4A1 | 4823149 | C | 3.22 | 4.04E−02 |
| PSD3 | 7001013 | C | 4.49 | 4.06E−02 |
| PPP1R9A | 7776891 | C | 5.52 | 4.07E−02 |
| QRFPR | 11098616 | A | 3.26 | 4.13E−02 |
| SLC35F3 | 4418678 | G | 5.94 | 4.16E−02 |
| CTNNA2 | 6711371 | G | 2.90 | 4.17E−02 |
| CREB5 | 714500 | A | 3.64 | 4.17E−02 |
| KIAA0182 | 11640338 | A | 2.80 | 4.22E−02 |
| GPC6 | 1924384 | A | 2.90 | 4.22E−02 |
| LINC00114 | 2836659 | A | 3.24 | 4.29E−02 |
| CSMD1 | 2449210 | C | 2.89 | 4.30E−02 |
| PDE10A | 515579 | A | 3.03 | 4.39E−02 |
| ARNT2 | 1020398 | C | 3.32 | 4.41E−02 |
| ATP2B2 | 42445 | A | 3.53 | 4.42E−02 |
| FMN2 | 10926139 | C | 3.60 | 4.47E−02 |
| ITPR1 | 9831960 | A | 3.15 | 4.48E−02 |
| NRXN3 | 17108086 | G | 3.25 | 4.53E−02 |
| WBSCR17 | 2158735 | C | 2.97 | 4.54E−02 |
| CTNNA2 | 450658 | C | 2.88 | 4.58E−02 |
| GNG2 | 1890699 | A | 3.22 | 4.58E−02 |
| PID1 | 3755302 | A | 3.70 | 4.66E−02 |
| ATP2B2 | 3774155 | A | 3.48 | 4.71E−02 |
| MAGI1 | 17432146 | C | 4.14 | 4.81E−02 |
| FBXO4 | 9292832 | A | 6.56 | 4.84E−02 |
| GOT2 | 11861897 | A | 10.70 | 4.88E−02 |
| TMEM132B | 16919368 | A | 6.87 | 4.90E−02 |
| CACNA2D1 | 258657 | C | 3.30 | 4.91E−02 |
| CREB5 | 1468447 | A | 3.38 | 4.91E−02 |
| LRP1B | 16847247 | A | 3.42 | 4.96E−02 |
| ARNTL | 10766074 | C | 4.48 | 4.97E−02 |
| CSMD1 | 688579 | A | 3.89 | 4.98E−02 |

### Example 3

### Novel Haplotype-Tagging SNPs Impacting Response for Perphenazine

Table 2A provides numerous examples of SNP alleles that predict good response to perphenazine, and table 2B provides numerous examples of SNP alleles that predict poor response to perphenazine. Tables 2A and 2B report the SNPs, SNP-alleles, P values, and Beta weights (in PANSS-Total units) from the linear regression for SNPs that affect response to perphenazine. A negative beta weight indicates that the allele is associated with a decrease in PANSS-T score, corresponding to greater improvement (or lowering) of symptom burden. A positive beta weight indicates that the allele is associated with an increase in PANSS-T score, corresponding to a worsening (or increase) of symptom burden.

TABLE 2A
Alleles Influencing Good Response to Perphenazine

| | | | Beta | |
|---|---|---|---|---|
| Gene | NCBI RS# | Allele | (PANSS) | P |
| MCPH1 | 11774231 | C | −17.25 | 7.72E−06 |
| ARNTL | 7126225 | C | −48.45 | 1.68E−05 |
| PRKCE | 2278773 | C | −24.74 | 1.69E−05 |
| MCPH1 | 17570753 | A | −14.24 | 2.25E−05 |
| CDH13 | 2116971 | C | −7.81 | 2.09E−04 |
| SKOR2 | 9952628 | G | −6.54 | 3.41E−04 |
| SKOR2 | 2247784 | T | −6.70 | 7.77E−04 |
| CACNA2D3 | 2139683 | A | −6.99 | 9.18E−04 |
| MACROD2 | 8117909 | A | −6.85 | 1.17E−03 |
| NAALADL2 | 9826737 | C | −12.68 | 1.21E−03 |
| NALCN | 8000980 | C | −9.07 | 1.39E−03 |
| ETV1 | 10236596 | G | −26.20 | 1.51E−03 |
| KATNAL2 | 9304340 | A | −6.42 | 1.77E−03 |
| CDH13 | 11647270 | A | −6.77 | 1.95E−03 |
| AMPH | 35024632 | G | −18.32 | 2.02E−03 |
| SAMD12 | 12550842 | A | −6.03 | 2.32E−03 |
| FERD3L | 10254337 | C | −7.16 | 2.78E−03 |
| CLSTN2 | 17397077 | A | −11.68 | 3.03E−03 |
| PLCXD2 | 4491869 | G | −8.82 | 3.28E−03 |
| SDK1 | 7812170 | A | −5.18 | 3.36E−03 |
| MACROD2 | 4813204 | G | −5.86 | 3.40E−03 |
| CACNA2D1 | 2299158 | C | −7.30 | 3.58E−03 |
| DYNC1I1 | 1488519 | C | −9.02 | 3.73E−03 |
| ITGAD | 4889654 | A | −6.12 | 3.79E−03 |
| STK31 | 6963309 | A | −6.41 | 3.82E−03 |
| SAMD12 | 13256262 | G | −5.69 | 3.96E−03 |
| NPAS3 | 10145961 | A | −5.50 | 4.10E−03 |
| PLCXD2 | 4325897 | A | −7.83 | 4.13E−03 |
| CCDC165 | 566890 | G | −8.56 | 4.38E−03 |
| CACNB2 | 2148184 | A | −8.11 | 4.54E−03 |
| HSPA12A | 17095095 | A | −10.43 | 4.72E−03 |
| CLSTN2 | 9852679 | C | −10.67 | 5.00E−03 |
| CDH10 | 12653077 | G | −7.40 | 5.03E−03 |
| FER1L6; FER1L6-AS1 | 7840702 | A | −9.95 | 5.14E−03 |
| DNAH17 | 4969188 | C | −6.96 | 5.15E−03 |
| FSTL5 | 2314105 | G | −14.83 | 5.87E−03 |
| RIBC2 | 738227 | A | −5.30 | 6.36E−03 |
| OPCML | 4937724 | A | −5.20 | 6.37E−03 |
| ERBB4 | 6435681 | A | −6.98 | 6.42E−03 |
| OPCML | 7110211 | G | −5.28 | 6.49E−03 |
| TRIO | 6554852 | A | −5.92 | 6.65E−03 |
| EPHB2 | 2165331 | A | −5.81 | 6.66E−03 |
| CDH13 | 8059696 | C | −6.18 | 6.77E−03 |
| NPY | 1859291 | A | −11.20 | 6.85E−03 |
| NPY | 3857723 | A | −11.20 | 6.85E−03 |
| KCNK10 | 10483994 | G | −11.75 | 7.02E−03 |
| PSMD14 | 9713 | A | −5.59 | 7.13E−03 |
| OPCML | 12796925 | A | −8.66 | 7.30E−03 |
| KCNA10 | 34970857 | C | −8.98 | 7.52E−03 |
| PLCB1 | 3761168 | G | −11.05 | 7.69E−03 |
| CCDC93 | 11556267 | A | −7.87 | 7.75E−03 |
| SCLT1 | 3113489 | A | −6.25 | 8.86E−03 |
| CSMD3 | 7018166 | G | −6.22 | 9.00E−03 |
| GFRA2 | 11990425 | C | −4.91 | 9.37E−03 |
| FHIT | 2736780 | C | −5.66 | 9.39E−03 |
| PTDSS1 | 2319815 | A | −5.10 | 9.51E−03 |
| RBFOX3 | 8074560 | A | −6.31 | 9.58E−03 |
| ATXN1 | 3116712 | A | −13.38 | 1.01E−02 |
| CA10 | 1503055 | G | −4.68 | 1.02E−02 |
| LYPD6 | 1196666 | C | −7.49 | 1.05E−02 |
| NAB1 | 1023568 | T | −4.61 | 1.05E−02 |
| NPAS3 | 8007455 | A | −5.13 | 1.07E−02 |
| GRB10 | 2329486 | A | −6.15 | 1.13E−02 |
| NEBL | 313788 | A | −7.16 | 1.14E−02 |
| NEDD4L | 292450 | C | −6.26 | 1.18E−02 |
| BAALC; LOC100499183 | 10099640 | C | −11.68 | 1.19E−02 |
| HTR1B | 2226183 | A | −8.13 | 1.20E−02 |
| NKAIN3 | 4379439 | C | −7.05 | 1.20E−02 |
| SLC18A2 | 363343 | A | −6.50 | 1.20E−02 |
| SLC18A2 | 363420 | C | −6.50 | 1.20E−02 |
| RIBC2 | 1022478 | C | −5.40 | 1.25E−02 |
| FSTL5 | 6829185 | G | −6.00 | 1.27E−02 |
| BLZF1 | 2275299 | C | −4.54 | 1.36E−02 |
| SYNRG | 7207076 | G | −4.62 | 1.37E−02 |
| MMP27 | 12099177 | A | −7.40 | 1.41E−02 |
| DLGAP2 | 7830545 | C | −6.27 | 1.42E−02 |
| MCPH1; ANGPT2 | 2242005 | A | −10.70 | 1.46E−02 |
| ACCN1 | 28936 | A | −4.88 | 1.50E−02 |
| INADL | 1286823 | A | −8.68 | 1.53E−02 |
| CNTN4 | 1153512 | A | −5.05 | 1.53E−02 |
| DYNC1I1 | 17638044 | C | −7.44 | 1.55E−02 |
| SHROOM3 | 4380545 | A | −5.77 | 1.63E−02 |
| GNG2 | 1253669 | G | −4.63 | 1.63E−02 |
| NBEA | 7325781 | A | −4.95 | 1.69E−02 |
| PACRG | 1001491 | C | −5.13 | 1.70E−02 |
| LINC00299 | 11904044 | C | −9.49 | 1.72E−02 |
| TSNAX-DISC1 | 1411776 | A | −6.54 | 1.76E−02 |
| PPARGC1A | 2970870 | C | −5.07 | 1.85E−02 |
| RASGRP1 | 4567661 | A | −4.50 | 1.88E−02 |
| CAST | 10515244 | C | −7.45 | 1.94E−02 |
| CAST | 11750400 | A | −7.45 | 1.94E−02 |
| WWOX | 16948667 | A | −7.20 | 1.94E−02 |
| PCSK6 | 735163 | C | −7.93 | 1.96E−02 |
| SYNE1 | 17082273 | G | −7.89 | 2.02E−02 |
| IKZF2 | 7607184 | C | −4.92 | 2.03E−02 |
| SDK1 | 17133426 | C | −5.37 | 2.05E−02 |
| FAM186A | 12809349 | C | −15.98 | 2.06E−02 |
| NRXN3 | 2199796 | A | −7.51 | 2.08E−02 |
| ATF6 | 1135983 | C | −8.60 | 2.09E−02 |
| LOC100505806 | 1614229 | A | −6.21 | 2.12E−02 |
| LOC100505806 | 1651285 | A | −6.21 | 2.12E−02 |
| CSMD1 | 3802296 | C | −6.00 | 2.20E−02 |
| CDH13 | 3844414 | A | −7.93 | 2.29E−02 |
| CCDC165 | 651219 | A | −5.12 | 2.30E−02 |
| RORA | 341400 | A | −8.45 | 2.33E−02 |
| CLSTN2 | 16850488 | C | −5.52 | 2.36E−02 |
| ATRN | 1064833 | C | −5.12 | 2.40E−02 |
| PTPRN2 | 896773 | A | −11.19 | 2.51E−02 |
| ST8SIA1 | 2160631 | C | −4.96 | 2.54E−02 |
| KCNB2 | 17762432 | A | −4.29 | 2.56E−02 |
| SKOR2 | 2137287 | A | −4.91 | 2.57E−02 |
| CERS5 | 2242507 | A | −4.58 | 2.57E−02 |
| RBFOX3 | 1077693 | C | −5.53 | 2.70E−02 |
| AKAP9 | 6960867 | A | −4.40 | 2.71E−02 |
| GNG2 | 1253671 | G | −4.31 | 2.79E−02 |
| GABRR2 | 3798256 | T | −3.73 | 2.82E−02 |
| FBLN7 | 3811643 | C | −5.64 | 2.92E−02 |
| FBLN7 | 4849050 | C | −5.64 | 2.92E−02 |
| PDE1C | 1115731 | A | −4.83 | 2.95E−02 |
| CSMD1 | 12549644 | A | −6.45 | 2.96E−02 |
| EPHB2 | 4655120 | A | −5.20 | 2.99E−02 |
| DLG2 | 1226063 | A | −5.35 | 3.00E−02 |
| RYR2 | 2065985 | T | −4.65 | 3.04E−02 |
| DGKI | 2113578 | C | −4.49 | 3.05E−02 |
| CD247 | 1052231 | A | −6.34 | 3.07E−02 |
| GRB10 | 11772525 | A | −5.26 | 3.12E−02 |
| SVEP1 | 7865430 | C | −7.14 | 3.18E−02 |
| COMMD1 | 7583942 | C | −4.97 | 3.24E−02 |
| MGAT2 | 1011373 | C | −8.70 | 3.26E−02 |
| NRCAM | 2284290 | C | −4.61 | 3.26E−02 |
| MAGI2 | 319863 | C | −4.63 | 3.30E−02 |
| NKAIN3 | 2353366 | G | −5.75 | 3.33E−02 |
| NCS1 | 11552451 | C | −6.39 | 3.34E−02 |
| ROBO1 | 17313732 | A | −4.38 | 3.37E−02 |
| PREX1 | 6095246 | A | −4.55 | 3.38E−02 |
| ARHGAP21 | 7092130 | C | −7.47 | 3.40E−02 |
| LOC100506689 | 611419 | A | −5.59 | 3.43E−02 |
| ZNF536 | 12972537 | G | −4.55 | 3.45E−02 |
| ZNF536 | 4805590 | C | −4.55 | 3.45E−02 |
| FHIT | 9831415 | C | −6.80 | 3.47E−02 |
| NALCN | 2390621 | C | −4.18 | 3.54E−02 |
| RYR2 | 12133002 | G | −4.46 | 3.55E−02 |
| FERD3L | 10268160 | C | −6.16 | 3.58E−02 |
| GPC6 | 8002366 | C | −7.79 | 3.60E−02 |
| CCDC50 | 6774730 | G | −8.06 | 3.62E−02 |
| ITPR1 | 4685810 | C | −4.72 | 3.63E−02 |
| ATRN | 2250106 | A | −4.24 | 3.67E−02 |
| ATRN | 2250338 | C | −4.24 | 3.67E−02 |
| MTUS2 | 9805210 | A | −6.73 | 3.69E−02 |
| ELOVL7 | 2219333 | A | −11.36 | 3.70E−02 |
| CHMP6 | 1128687 | C | −4.13 | 3.70E−02 |
| GABRR2 | 10944441 | A | −6.04 | 3.71E−02 |
| DCAF11 | 3825583 | A | −9.18 | 3.72E−02 |
| C19orf45 | 475923 | A | −4.18 | 3.80E−02 |
| CDH13 | 17701213 | C | −6.29 | 3.82E−02 |
| LEPREL1 | 9290920 | A | −4.86 | 3.92E−02 |
| NBEA | 12871646 | A | −8.23 | 3.98E−02 |
| MACROD2 | 6080110 | G | −3.92 | 3.99E−02 |
| PTPN5 | 7946254 | C | −14.23 | 4.00E−02 |
| CELF2 | 7091228 | A | −4.40 | 4.06E−02 |
| MUC7 | 6826961 | C | −5.13 | 4.10E−02 |
| TRPC4 | 9603241 | A | −5.08 | 4.13E−02 |
| SGCZ | 6530806 | G | −5.20 | 4.20E−02 |
| LINC00308 | 2827687 | C | −6.71 | 4.23E−02 |
| CSMD1 | 2623630 | A | −4.35 | 4.27E−02 |
| SGCZ | 1480696 | C | −4.91 | 4.29E−02 |
| SLC17A8 | 11568537 | C | −4.11 | 4.30E−02 |
| ACYP2 | 843719 | A | −4.02 | 4.30E−02 |
| CNTNAP2 | 17171006 | C | −17.03 | 4.34E−02 |
| DLC1 | 6981968 | A | −4.32 | 4.49E−02 |
| PTGER3 | 5675 | C | −5.23 | 4.50E−02 |
| MYO5B | 12457962 | A | −8.39 | 4.54E−02 |
| PTPRG | 3817458 | C | −5.54 | 4.56E−02 |
| MAGI2 | 38108 | C | −4.37 | 4.61E−02 |
| DNAH9 | 12453566 | G | −5.40 | 4.62E−02 |
| DNAH9 | 2058039 | C | −5.40 | 4.62E−02 |
| ATRNL1 | 4751924 | C | −3.70 | 4.68E−02 |
| RYR3 | 2676023 | C | −5.04 | 4.70E−02 |
| ROBO2 | 1470571 | C | −4.26 | 4.73E−02 |
| NRXN3 | 2219848 | A | −4.11 | 4.80E−02 |
| FHIT | 17396765 | C | −6.22 | 4.81E−02 |
| CHMP6 | 1128705 | C | −3.91 | 4.81E−02 |
| SGCZ | 10095307 | A | −5.03 | 4.84E−02 |
| TBC1D2B | 4886989 | C | −4.78 | 4.84E−02 |
| WWOX | 2062894 | C | −3.63 | 4.85E−02 |
| ELFN2 | 1076934 | A | −3.35 | 4.87E−02 |
| PCSK6 | 900414 | A | −4.77 | 4.88E−02 |
| PLCG2 | 4243215 | A | −4.99 | 4.93E−02 |
| OPCML | 7944972 | A | −3.86 | 4.94E−02 |
| PARK2 | 6455765 | A | −4.75 | 4.98E−02 |

TABLE 2B
Alleles Influencing Poor Response to Perphenazine

| Gene | NCBI RS# | Allele | Beta (PANSS) | P |
|---|---|---|---|---|
| MAML3 | 11100483 | A | 7.28 | 4.83E−04 |
| GABBR2 | 2808534 | A | 7.07 | 8.05E−04 |
| CSMD1 | 7813376 | C | 6.19 | 1.24E−03 |
| PIP5K1B | 953390 | C | 6.34 | 2.02E−03 |
| CSMD1 | 6558885 | A | 8.38 | 2.28E−03 |
| GABBR2 | 1177531 | A | 7.09 | 2.71E−03 |
| SPINK1 | 4705203 | A | 7.81 | 2.75E−03 |
| UNC13C | 574138 | A | 6.27 | 3.03E−03 |
| ROBO1 | 444598 | C | 5.84 | 3.36E−03 |
| NBAS | 10929356 | G | 5.46 | 4.91E−03 |
| COL4A3; LOC654841 | 12464886 | C | 6.69 | 4.95E−03 |
| CADPS2 | 718764 | A | 6.75 | 5.16E−03 |
| NTSR2 | 7602721 | C | 7.29 | 5.82E−03 |
| ROBO1 | 6762005 | T | 5.47 | 6.09E−03 |
| CLSTN2 | 2042705 | C | 6.23 | 6.24E−03 |
| NKAIN3 | 10448028 | C | 5.85 | 6.87E−03 |
| NRXN3 | 2216901 | C | 6.60 | 6.93E−03 |
| KLHL32 | 850587 | T | 5.44 | 7.58E−03 |
| RNF144B | 6927583 | G | 8.72 | 8.25E−03 |
| PLCB1 | 6086680 | A | 5.81 | 8.35E−03 |
| CA10 | 17607202 | C | 5.47 | 8.38E−03 |
| PARK2 | 9458561 | C | 5.53 | 9.33E−03 |
| NBAS | 12692258 | A | 6.21 | 9.35E−03 |
| CDH13 | 6565109 | A | 5.12 | 9.39E−03 |
| FGF14 | 3007763 | A | 5.71 | 9.50E−03 |
| TSPAN13 | 3807509 | C | 4.75 | 9.53E−03 |
| MTSS1 | 6999708 | C | 5.72 | 9.71E−03 |
| PHIP | 9350797 | A | 5.51 | 1.01E−02 |
| KCNQ1; KCNQ1OT1 | 231358 | C | 4.98 | 1.10E−02 |
| CTNND2 | 6875838 | A | 5.02 | 1.14E−02 |
| FLJ35024 | 875587 | A | 6.14 | 1.14E−02 |
| GNAS | 6064716 | C | 8.71 | 1.21E−02 |
| MAGI1 | 17073748 | A | 7.64 | 1.29E−02 |
| RAB6B | 7644124 | C | 5.16 | 1.33E−02 |
| NPAS3 | 10135876 | C | 5.11 | 1.38E−02 |
| ZFPM2 | 2920048 | C | 7.44 | 1.48E−02 |
| DLG2 | 2512676 | G | 5.77 | 1.49E−02 |
| KCNQ3 | 10097662 | A | 6.38 | 1.55E−02 |
| PCDH10 | 11731618 | G | 5.70 | 1.56E−02 |
| PCDH10 | 13130047 | A | 5.70 | 1.56E−02 |
| KCNB2 | 17828687 | A | 4.68 | 1.59E−02 |
| PCLO | 2877 | C | 5.03 | 1.61E−02 |
| MYT1L | 17338616 | A | 4.98 | 1.62E−02 |
| NECAB1 | 1055146 | C | 5.00 | 1.63E−02 |
| DOK5 | 2840 | C | 6.51 | 1.63E−02 |
| CA10 | 1909923 | T | 4.41 | 1.68E−02 |
| SMARCA2 | 10081778 | C | 5.32 | 1.71E−02 |
| NBEA | 10507424 | A | 5.28 | 1.72E−02 |
| SLC7A14 | 4955729 | C | 4.45 | 1.73E−02 |
| AGL | 2307129 | A | 6.78 | 1.73E−02 |
| NPAS3 | 1952595 | A | 4.95 | 1.80E−02 |
| GABBR2 | 12552470 | A | 6.50 | 1.84E−02 |
| KCNMA1 | 11002064 | C | 4.58 | 1.89E−02 |
| COL4A3; LOC654841 | 4603754 | C | 5.01 | 1.91E−02 |
| KCNQ1 | 2237869 | A | 5.73 | 1.92E−02 |
| MACROD2 | 12480304 | G | 6.03 | 1.97E−02 |
| FERMT1 | 2295434 | A | 4.89 | 1.98E−02 |
| FLJ35024 | 693934 | A | 4.79 | 1.99E−02 |
| MAP1B | 10062773 | A | 4.46 | 2.12E−02 |
| CNOT2 | 7969013 | C | 5.27 | 2.17E−02 |
| PDE4D | 16889901 | A | 4.77 | 2.20E−02 |
| LOC100129434 | 2216327 | C | 9.55 | 2.20E−02 |
| CA10 | 7210687 | C | 4.35 | 2.24E−02 |
| KCNB1 | 3331 | C | 11.40 | 2.24E−02 |
| SGCZ | 12114757 | C | 6.02 | 2.27E−02 |
| ELMOD1 | 10890742 | A | 4.79 | 2.28E−02 |
| NEDD9 | 6933985 | C | 5.45 | 2.28E−02 |
| NKAIN3 | 7388305 | C | 4.89 | 2.31E−02 |
| CA10 | 1503056 | G | 4.35 | 2.38E−02 |
| ACCS | 178506 | A | 4.48 | 2.38E−02 |
| MEPE | 3749575 | A | 7.44 | 2.42E−02 |
| KCNIP4 | 16870818 | G | 4.73 | 2.50E−02 |
| NRXN3 | 9671249 | C | 6.47 | 2.53E−02 |
| FMN2 | 10926208 | A | 5.97 | 2.54E−02 |
| DGKB | 10240641 | A | 4.26 | 2.56E−02 |
| MAGI2 | 12668963 | A | 4.68 | 2.61E−02 |
| NRXN3 | 221516 | A | 8.25 | 2.61E−02 |
| ATP2B2 | 11708983 | A | 5.93 | 2.62E−02 |
| PACRG | 6922278 | C | 4.15 | 2.83E−02 |
| MACROD2 | 6043223 | C | 5.97 | 2.88E−02 |
| KCNMA1 | 7907070 | A | 4.22 | 2.92E−02 |
| EMID2 | 10447812 | A | 11.83 | 2.96E−02 |
| CSMD1 | 2948644 | T | 4.04 | 2.99E−02 |
| LOC100505985 | 9349534 | T | 4.60 | 3.02E−02 |
| KCNQ1 | 11023485 | A | 4.50 | 3.10E−02 |
| SPINK1 | 10035432 | A | 5.44 | 3.11E−02 |
| SLC22A16 | 3778650 | A | 5.11 | 3.19E−02 |
| EPAS1 | 13428739 | C | 7.26 | 3.22E−02 |
| TSPAN9 | 7980107 | C | 4.46 | 3.29E−02 |
| PARK2 | 2022996 | A | 5.01 | 3.31E−02 |
| PRKG1 | 10998789 | A | 4.58 | 3.33E−02 |
| TRPM3 | 1336380 | G | 4.43 | 3.34E−02 |
| PIK3C2G | 10770363 | A | 3.86 | 3.35E−02 |
| TSPAN9 | 7308849 | C | 4.43 | 3.36E−02 |
| VCAN | 3797782 | A | 4.40 | 3.37E−02 |
| NRG3 | 10509433 | C | 8.87 | 3.41E−02 |
| ATP2B2 | 11712897 | C | 5.68 | 3.44E−02 |
| RGS7 | 261827 | C | 4.13 | 3.50E−02 |
| CA10 | 203032 | C | 4.12 | 3.51E−02 |
| NAV2 | 2625323 | G | 5.73 | 3.54E−02 |
| EXOC2 | 17134263 | C | 7.56 | 3.57E−02 |
| NBEA | 9530787 | G | 4.50 | 3.60E−02 |
| GABBR2 | 2808532 | A | 6.15 | 3.62E−02 |
| PTPRT | 208243 | A | 3.99 | 3.63E−02 |
| NBAS | 10179251 | A | 5.22 | 3.71E−02 |
| PON1 | 705382 | C | 4.00 | 3.76E−02 |
| MACROD2 | 6043211 | C | 5.78 | 3.76E−02 |
| KCNB2 | 4738266 | C | 4.46 | 3.87E−02 |
| SLC1A1 | 17755777 | C | 4.61 | 3.88E−02 |
| FAM104A | 4969023 | C | 8.27 | 3.90E−02 |
| RYR2 | 1362840 | G | 4.46 | 3.98E−02 |
| ASTN2 | 1321922 | C | 5.15 | 3.99E−02 |
| KCNQ3 | 2597351 | C | 5.95 | 4.00E−02 |
| ITPR1 | 11705928 | A | 4.79 | 4.05E−02 |
| ITPR1 | 9809124 | A | 4.79 | 4.05E−02 |
| DPP10 | 36044503 | A | 5.73 | 4.06E−02 |
| CCDC165 | 663178 | T | 3.95 | 4.14E−02 |
| IGSF22 | 4237729 | C | 4.39 | 4.19E−02 |
| CA10 | 9900811 | T | 3.82 | 4.24E−02 |
| NCAM2 | 2826890 | G | 4.00 | 4.24E−02 |
| GRHL2 | 11787301 | A | 4.85 | 4.33E−02 |
| SHC3 | 4877046 | A | 5.02 | 4.37E−02 |
| PDE1C | 30602 | A | 3.96 | 4.38E−02 |
| ESRRG | 9441544 | C | 4.86 | 4.38E−02 |
| CTNNA2 | 13019601 | A | 3.95 | 4.40E−02 |
| GRID2 | 17330509 | C | 3.84 | 4.43E−02 |
| SLC4A1AP | 9678851 | A | 4.04 | 4.51E−02 |
| ERC2 | 4974115 | C | 3.89 | 4.53E−02 |
| STXBP5L | 7618583 | C | 7.00 | 4.56E−02 |
| PCDH7 | 13150083 | A | 4.48 | 4.59E−02 |
| ITPR1 | 1873850 | C | 4.66 | 4.77E−02 |
| PACRG | 6455871 | C | 3.83 | 4.83E−02 |
| NPAS3 | 10134571 | A | 5.63 | 4.95E−02 |
| PIKFYVE | 12622556 | A | 5.03 | 4.97E−02 |

### Example 4

### Novel Haplotype-Tagging SNPs Impacting Response for Quetiapine

Table 3A provides numerous examples of SNP alleles that predict good response to quetiapine, and table 3B provides numerous examples of SNP alleles that predict poor response to quetiapine. Tables 3A and 3B report the SNPs, SNP-alleles, P values, and Beta weights (in PANSS-Total units) from the linear regression for SNPs that affect response to quetiapine. A negative beta weight indicates that the allele is associated with a decrease in PANSS-T score, corresponding to greater improvement (or lowering) of symptom burden. A positive beta weight indicates that the allele is associated with an increase in PANSS-T score, corresponding to a worsening (or increase) of symptom burden.

TABLE 3A
Alleles Influencing Good Response to
Quetiapine

| Gene | NCBI RS# | Allele | Beta (PANSS) | P |
|---|---|---|---|---|
| HTR1B | 2226183 | A | −7.38 | 7.98E−04 |
| NLGN1 | 6772978 | A | −7.25 | 9.98E−04 |
| CNTN4 | 4684366 | C | −5.69 | 1.32E−03 |
| GREM2 | 9697 | C | −6.83 | 1.46E−03 |
| FHIT | 12636662 | A | −4.62 | 2.18E−03 |
| PCDH15 | 2384470 | C | −5.21 | 2.33E−03 |
| ARSB | 25414 | C | −9.37 | 2.37E−03 |
| COL22A1 | 4311658 | G | −5.04 | 2.37E−03 |
| COL22A1 | 4442151 | C | −5.04 | 2.37E−03 |
| DYNC1I1 | 17638044 | C | −8.72 | 2.39E−03 |
| RGS7 | 2678780 | A | −4.56 | 2.47E−03 |
| ETV1 | 41505 | C | −4.08 | 2.62E−03 |
| GRID2 | 1433667 | C | −4.87 | 2.84E−03 |
| ETV1 | 17167676 | G | −5.27 | 2.86E−03 |
| PTGER3 | 5675 | C | −6.55 | 2.95E−03 |
| NALCN | 12877625 | C | −4.40 | 2.97E−03 |
| CNTNAP2 | 7804277 | A | −4.78 | 3.01E−03 |
| LDB2 | 872478 | C | −5.04 | 3.22E−03 |
| NPFFR2 | 11940196 | A | −4.66 | 3.56E−03 |
| HTR1B | 11568817 | G | −4.41 | 3.57E−03 |
| ETV1 | 2073532 | C | −4.97 | 3.58E−03 |
| ETV1 | 2073533 | A | −4.97 | 3.58E−03 |
| ETV1 | 41506 | A | −4.97 | 3.58E−03 |
| LRP1B | 4954861 | C | −4.43 | 3.63E−03 |
| TMEFF2 | 3768703 | C | −4.56 | 3.70E−03 |
| ARMC3 | 7098947 | A | −7.36 | 3.91E−03 |
| L3MBTL4 | 3811 | A | −4.75 | 3.99E−03 |
| PLCG2 | 12925104 | C | −5.09 | 4.24E−03 |
| PSD3 | 17595804 | A | −9.06 | 4.33E−03 |
| PPP2R2B | 17524553 | A | −6.53 | 4.36E−03 |
| DCC | 11875845 | C | −5.85 | 4.66E−03 |
| CSMD1 | 17066296 | A | −7.67 | 4.71E−03 |
| TMEFF2 | 4853493 | G | −4.70 | 4.75E−03 |
| DOK6 | 12960929 | A | −4.43 | 5.25E−03 |
| SLC6A1 | 1170694 | A | −5.04 | 5.46E−03 |
| NLGN1 | 1980298 | C | −4.13 | 5.83E−03 |
| FHIT | 4145506 | A | −4.76 | 6.05E−03 |
| EPHA6 | 13080770 | C | −5.82 | 6.11E−03 |
| FHIT | 398105 | A | −3.75 | 6.22E−03 |
| LRP1B | 10496858 | C | −4.35 | 6.35E−03 |
| ERC2 | 4974115 | C | −4.27 | 6.62E−03 |
| DCTN4 | 11954652 | C | −8.63 | 6.67E−03 |
| MACROD2 | 6514537 | T | −3.87 | 6.83E−03 |
| RGS7 | 1878729 | C | −4.01 | 7.24E−03 |
| SVEP1 | 1410045 | A | −5.03 | 7.49E−03 |
| RORA | 8037420 | C | −4.03 | 8.07E−03 |
| TRAPPC10 | 2838476 | C | −5.10 | 8.67E−03 |
| CA10 | 203032 | C | −3.83 | 8.90E−03 |
| DNAH5 | 4549527 | A | −6.52 | 9.33E−03 |
| BNIP2 | 1057059 | G | −4.03 | 9.40E−03 |
| NRG3 | 1937972 | A | −4.41 | 9.75E−03 |
| SEPT9 | 2627222 | C | −14.60 | 9.78E−03 |
| KYNU | 351678 | G | −4.09 | 9.95E−03 |
| MSR1 | 12675467 | C | −8.89 | 1.00E−02 |
| MSR1 | 918 | A | −8.89 | 1.00E−02 |
| CDH7 | 2291343 | A | −4.21 | 1.01E−02 |
| TLN2 | 16945912 | C | −13.51 | 1.04E−02 |
| ZNF532 | 3737506 | G | −10.25 | 1.04E−02 |
| PHIP | 9350797 | A | −4.32 | 1.04E−02 |
| GFRA1 | 2694766 | A | −3.82 | 1.07E−02 |
| CNTNAP2 | 6957194 | A | −4.24 | 1.10E−02 |
| SEPT9 | 448767 | A | −14.90 | 1.11E−02 |
| CCDC93 | 17569222 | C | −8.74 | 1.13E−02 |
| CNTN4 | 767460 | A | −4.52 | 1.15E−02 |
| CSMD1 | 10503264 | A | −4.17 | 1.15E−02 |
| NCS1 | 887534 | G | −5.89 | 1.17E−02 |
| LOC286094 | 7007485 | A | −4.14 | 1.17E−02 |
| CACNA2D1 | 7811209 | A | −4.74 | 1.18E−02 |
| KCNIP4 | 2011495 | C | −4.09 | 1.20E−02 |
| MYO10 | 27878 | A | −3.71 | 1.25E−02 |
| OPCML | 7940198 | A | −5.30 | 1.28E−02 |
| GALNTL4 | 4910339 | A | −6.50 | 1.37E−02 |
| DGKB | 2024038 | C | −4.71 | 1.37E−02 |
| DLG2 | 10898304 | C | −3.65 | 1.41E−02 |
| DYNC1I1 | 1488519 | C | −7.20 | 1.48E−02 |
| NRG3 | 11194491 | C | −3.99 | 1.52E−02 |
| MSI2 | 9912674 | A | −4.89 | 1.55E−02 |
| ATF6 | 1135983 | C | −7.15 | 1.56E−02 |
| RYR3 | 12906601 | C | −5.99 | 1.56E−02 |
| GPR97 | 7193154 | C | −5.36 | 1.57E−02 |
| THADA | 6754589 | A | −4.13 | 1.57E−02 |
| SLCO3A1 | 12708589 | C | −3.72 | 1.57E−02 |
| PARK2 | 9365365 | C | −4.64 | 1.61E−02 |
| CNTNAP2 | 17170957 | G | −4.57 | 1.63E−02 |
| PIK3CG | 3173908 | C | −4.53 | 1.67E−02 |
| GRM5 | 3824927 | G | −3.45 | 1.70E−02 |
| KCNQ1 | 11022855 | C | −6.87 | 1.75E−02 |
| PARD3B | 2197691 | A | −4.14 | 1.80E−02 |
| NALCN | 12874108 | A | −3.89 | 1.87E−02 |
| NALCN | 9513862 | C | −3.89 | 1.87E−02 |
| ARHGAP15 | 9287353 | C | −3.91 | 1.94E−02 |
| CDH7 | 12607785 | A | −3.69 | 1.94E−02 |
| CDH7 | 4580293 | C | −3.69 | 1.94E−02 |
| DOK6 | 7228021 | G | −3.57 | 1.95E−02 |
| LRP1B | 2380943 | C | −3.47 | 1.96E−02 |
| TRIM9 | 882413 | C | −4.35 | 2.00E−02 |
| HAAO | 3755540 | C | −3.79 | 2.01E−02 |
| CDH13 | 8048616 | C | −3.49 | 2.03E−02 |
| PTPRT | 876523 | C | −3.45 | 2.06E−02 |
| CACNA2D3 | 9861155 | A | −4.28 | 2.07E−02 |
| INMT- | 10230286 | A | −3.51 | 2.09E−02 |
| FAM188B; | | | | |
| FAM188B | | | | |
| CTNNA2 | 10520244 | C | −6.07 | 2.12E−02 |
| RIMBP2 | 12305517 | C | −5.62 | 2.16E−02 |
| C13orf35 | 4907727 | C | −3.58 | 2.16E−02 |
| MSR1 | 6981231 | C | −7.67 | 2.17E−02 |
| FMNL2 | 1155779 | C | −3.41 | 2.20E−02 |
| SDK2 | 4969114 | A | −3.33 | 2.22E−02 |
| WWOX | 7203676 | C | −3.87 | 2.23E−02 |
| CPLX2 | 2243404 | C | −4.30 | 2.25E−02 |
| MYO10 | 26317 | A | −3.80 | 2.25E−02 |
| CNTN6 | 7646412 | A | −4.64 | 2.28E−02 |
| CTNNA2 | 10201249 | A | −4.77 | 2.33E−02 |
| OPCML | 3920986 | G | −3.72 | 2.33E−02 |
| KCNB1 | 34467662 | C | −9.06 | 2.39E−02 |
| ESRRG | 9441544 | C | −4.04 | 2.40E−02 |
| TMC1 | 10869183 | T | −3.43 | 2.41E−02 |
| MCPH1 | 2515509 | C | −5.16 | 2.44E−02 |
| SHC3 | 10122011 | C | −3.43 | 2.47E−02 |
| GALNTL4 | 4909979 | C | −5.30 | 2.49E−02 |
| CA10 | 7210687 | C | −3.24 | 2.52E−02 |
| OPCML | 3018407 | A | −3.48 | 2.60E−02 |
| GRM8 | 17865066 | A | −10.79 | 2.61E−02 |
| GRM8 | 17865434 | C | −10.79 | 2.61E−02 |
| KCNQ3 | 17659416 | A | −4.39 | 2.67E−02 |
| NLGN1 | 2861338 | A | −3.51 | 2.69E−02 |
| INMT- | 11972565 | A | −3.53 | 2.70E−02 |
| FAM188B; | | | | |
| FAM188B | | | | |
| DENND4C | 2666797 | C | −3.79 | 2.72E−02 |
| DGKD | 838717 | A | −3.38 | 2.74E−02 |
| ATP2B2 | 11712897 | C | −3.54 | 2.77E−02 |
| LRP1B | 34488772 | A | −8.41 | 2.80E−02 |
| PRKG1 | 1904018 | C | −3.09 | 2.81E−02 |
| CERK | 78424 | A | −3.60 | 2.86E−02 |
| C15orf41 | 8027206 | A | −3.40 | 2.90E−02 |
| NRG3 | 12767532 | G | −3.66 | 2.93E−02 |
| CTNNA2 | 17018593 | A | −4.25 | 2.95E−02 |
| GRID2 | 1836115 | A | −3.53 | 3.01E−02 |
| LRFN2 | 2078460 | A | −3.67 | 3.06E−02 |
| OPCML | 3019855 | C | −3.40 | 3.16E−02 |
| KCNIP4 | 10516363 | A | −3.43 | 3.20E−02 |
| STX11 | 3734228 | C | −9.67 | 3.21E−02 |
| STX11 | 6912580 | C | −9.67 | 3.21E−02 |
| SORBS1 | 4572071 | A | −3.66 | 3.25E−02 |
| NPAS3 | 927763 | G | −4.45 | 3.26E−02 |
| ST8SIA1 | 4606528 | C | −3.84 | 3.26E−02 |
| DLC1 | 12677920 | A | −3.35 | 3.27E−02 |
| SLC35F3 | 12087906 | G | −9.04 | 3.30E−02 |
| FOXP1 | 831440 | C | −3.67 | 3.37E−02 |
| CCBE1 | 12606241 | G | −4.12 | 3.38E−02 |
| PLCG2 | 4889447 | C | −5.77 | 3.48E−02 |
| TMTC2 | 7974520 | A | −3.64 | 3.49E−02 |
| ROBO2 | 1470571 | C | −3.69 | 3.50E−02 |
| CHRM3 | 4659552 | A | −3.02 | 3.50E−02 |
| RGS7 | 10802943 | C | −2.99 | 3.52E−02 |
| JAG1 | 6074164 | A | −4.59 | 3.54E−02 |
| EXOC2 | 17135234 | A | −4.93 | 3.55E−02 |
| SAMD4A | 8011374 | C | −3.17 | 3.60E−02 |
| CNTNAP2 | 10240221 | A | −3.22 | 3.63E−02 |
| ITPR2 | 11048710 | A | −3.75 | 3.74E−02 |
| ERG | 10854385 | A | −4.00 | 3.76E−02 |
| GPR116 | 9395218 | C | −4.73 | 3.77E−02 |
| FMN2 | 1020709 | C | −3.38 | 3.77E−02 |
| FLJ38109 | 11167681 | C | −3.35 | 3.82E−02 |
| KCNQ1 | 422314 | C | −3.18 | 3.83E−02 |
| USP10 | 2012708 | C | −3.05 | 3.86E−02 |
| CA10 | 17607202 | C | −3.10 | 3.87E−02 |
| SGCZ | 12155874 | A | −3.08 | 3.89E−02 |
| LDB2 | 3805320 | A | −3.16 | 3.90E−02 |
| TGIF1 | 12606927 | A | −3.49 | 4.02E−02 |
| FBXL2 | 6772365 | C | −3.45 | 4.07E−02 |
| PARD3B | 7589114 | C | −3.18 | 4.08E−02 |
| TSC1 | 1050700 | A | −3.29 | 4.13E−02 |
| GRID2 | 13103135 | C | −3.66 | 4.20E−02 |
| IYD | 2076286 | A | −6.69 | 4.24E−02 |
| CLSTN2 | 10513102 | C | −4.05 | 4.28E−02 |
| RPRD1A | 4799835 | A | −3.76 | 4.31E−02 |
| SORBS1 | 4918911 | C | −3.34 | 4.32E−02 |
| GNG2 | 1343870 | G | −3.12 | 4.37E−02 |
| NCAM2 | 12483671 | A | −3.47 | 4.38E−02 |
| EXOC2 | 1747599 | G | −3.71 | 4.44E−02 |
| AKAP9 | 35759833 | C | −4.62 | 4.46E−02 |
| DLG2 | 7101982 | C | −2.93 | 4.47E−02 |
| CDH13 | 3935908 | G | −2.89 | 4.47E−02 |
| PLXDC2 | 989767 | A | −3.31 | 4.48E−02 |
| CSMD1 | 2616996 | G | −3.44 | 4.49E−02 |
| PIKFYVE | 17699982 | A | −4.12 | 4.51E−02 |
| CSMD1 | 2724971 | A | −3.06 | 4.51E−02 |
| OSBPL1A | 275861 | C | −3.06 | 4.55E−02 |
| RBFOX1 | 7194775 | C | −2.88 | 4.58E−02 |
| MYT1L | 17338616 | A | −2.88 | 4.58E−02 |
| CSMD1 | 2740865 | C | −3.52 | 4.60E−02 |
| IL1RAP | 2361835 | A | −4.37 | 4.63E−02 |
| IL1RAP | 7642607 | A | −4.37 | 4.63E−02 |
| IL1RAP | 7642797 | A | −4.37 | 4.63E−02 |
| FAM186A | 12809349 | C | −10.53 | 4.68E−02 |
| CCDC85A | 6704684 | A | −3.94 | 4.68E−02 |
| RYR2 | 17626494 | A | −3.72 | 4.68E−02 |
| NAV3 | 1731723 | C | −4.81 | 4.69E−02 |
| HYDIN | 1774423 | C | −4.48 | 4.70E−02 |
| CSMD1 | 2617002 | A | −3.54 | 4.71E−02 |
| MACROD2 | 1233774 | A | −3.02 | 4.77E−02 |
| FMN2 | 9729725 | C | −3.65 | 4.81E−02 |
| ITGA1 | 1047483 | A | −3.26 | 4.82E−02 |
| ITGA1 | 6895049 | G | −3.26 | 4.82E−02 |
| MSI2 | 9905296 | C | −4.56 | 4.85E−02 |
| CSMD1 | 2724973 | A | −4.25 | 4.86E−02 |
| WBSCR17 | 10238468 | C | −3.15 | 4.88E−02 |
| CTNND2 | 13362481 | C | −3.24 | 4.90E−02 |
| NRG3 | 585597 | C | −3.13 | 4.91E−02 |
| SORBS1 | 955759 | A | −3.24 | 4.95E−02 |
| SNCA | 356198 | A | −3.59 | 4.96E−02 |
| ARHGAP21 | 7090524 | A | −3.39 | 4.98E−02 |

TABLE 3B
Alleles Influencing Poor Response to Quetiapine

| Gene | NCBI RS# | Allele | Beta (PANSS) | P |
|---|---|---|---|---|
| KCNMA1 | 35793 | C | 13.23 | 1.81E−04 |
| SORBS1 | 11188339 | C | 6.46 | 6.12E−04 |
| NPAS3 | 10148780 | C | 5.96 | 1.11E−03 |
| NRG3 | 1739780 | A | 6.18 | 1.28E−03 |
| NPAS3 | 8013252 | C | 5.97 | 1.69E−03 |
| SORBS1 | 10882612 | C | 5.96 | 1.73E−03 |
| GRIN3A | 4324970 | C | 4.65 | 2.08E−03 |
| ROBO2 | 13323053 | A | 4.24 | 2.78E−03 |
| NAV2 | 2625319 | A | 4.76 | 2.80E−03 |
| COL4A4 | 1320407 | G | 4.69 | 2.90E−03 |
| NAV2 | 2585757 | G | 5.09 | 3.46E−03 |
| MYO3B | 33962844 | A | 5.07 | 3.65E−03 |
| CSMD1 | 4875309 | C | 5.79 | 3.69E−03 |
| LOC100289230 | 4703054 | A | 4.73 | 4.63E−03 |
| DOK6 | 12456201 | C | 4.39 | 4.71E−03 |
| DGKB | 13242030 | C | 7.37 | 5.07E−03 |
| GRIN3A | 2485528 | T | 4.23 | 5.60E−03 |
| ADTRP | 6929400 | C | 4.67 | 5.63E−03 |
| ADAMTS9 | 7615657 | C | 4.25 | 5.72E−03 |
| CA10 | 1503055 | G | 3.85 | 6.35E−03 |
| PTPRT | 6102904 | C | 6.55 | 6.49E−03 |
| CA10 | 1909923 | C | 3.84 | 6.67E−03 |
| CSMD1 | 2948644 | G | 4.15 | 6.79E−03 |
| ANK3 | 3897459 | C | 4.50 | 6.92E−03 |
| CA10 | 9900811 | G | 3.96 | 6.94E−03 |
| KAZN | 4661549 | C | 3.91 | 7.84E−03 |
| GPM6A | 2333250 | A | 5.13 | 7.98E−03 |
| PTPRG | 17066238 | G | 8.98 | 7.98E−03 |
| NRP2 | 11678877 | A | 4.07 | 8.69E−03 |
| MGAT2 | 1011373 | C | 12.58 | 9.21E−03 |
| NLGN1 | 4894648 | A | 4.76 | 9.80E−03 |
| BTN3A1 | 10447391 | G | 5.08 | 9.94E−03 |
| BTN3A1 | 3208734 | C | 5.08 | 9.94E−03 |
| BTN3A1 | 4711109 | A | 5.08 | 9.94E−03 |
| PLA2G2D | 578459 | A | 3.79 | 1.01E−02 |
| NLGN1 | 1421422 | A | 4.73 | 1.03E−02 |
| PRICKLE2 | 695938 | C | 6.98 | 1.04E−02 |
| MAGI2 | 740967 | C | 4.68 | 1.21E−02 |
| PCSK6 | 12910197 | A | 4.96 | 1.24E−02 |
| CLSTN2 | 1346134 | C | 4.60 | 1.29E−02 |
| MACROD2 | 6034267 | C | 5.13 | 1.36E−02 |
| RIBC2 | 1022478 | C | 3.91 | 1.41E−02 |
| CRISPLD1 | 17295835 | C | 4.29 | 1.42E−02 |
| FHIT | 602197 | C | 4.42 | 1.43E−02 |
| TFB1M | 428447 | C | 4.05 | 1.47E−02 |
| PDE4D | 1995166 | C | 3.62 | 1.48E−02 |
| EPHB1 | 2400398 | A | 4.93 | 1.48E−02 |
| DUOX2 | 2554452 | A | 3.94 | 1.58E−02 |
| ATXN1 | 2237186 | C | 5.75 | 1.60E−02 |
| SMEK2 | 3748945 | A | 4.30 | 1.62E−02 |
| NELL1 | 1793004 | C | 4.10 | 1.64E−02 |
| ERG | 2226375 | C | 4.18 | 1.64E−02 |
| CACNA2D1 | 10954673 | C | 4.83 | 1.64E−02 |
| SMARCA2 | 16937852 | C | 3.77 | 1.65E−02 |
| PRKCE | 7601378 | A | 4.08 | 1.70E−02 |
| PJA2 | 33730 | A | 3.50 | 1.72E−02 |
| ZFPM2 | 16873402 | C | 4.01 | 1.72E−02 |
| EMID2 | 10228469 | C | 3.62 | 1.73E−02 |
| SFRP1 | 17574424 | C | 4.16 | 1.78E−02 |
| ACCN1 | 28936 | A | 3.93 | 1.87E−02 |
| CSMD1 | 2720811 | A | 3.85 | 1.91E−02 |
| MAGI2 | 2885559 | C | 3.66 | 1.94E−02 |
| GALNT9 | 11246993 | A | 3.98 | 2.00E−02 |
| SOBP | 10554 | A | 5.23 | 2.00E−02 |
| CDH13 | 3743621 | C | 5.41 | 2.11E−02 |
| PLCG2 | 11863650 | A | 3.64 | 2.15E−02 |
| MACROD2 | 204093 | C | 3.65 | 2.15E−02 |
| UNC13C | 2456976 | C | 3.77 | 2.18E−02 |
| RNF144B | 17626032 | A | 4.32 | 2.18E−02 |
| CREB3L2 | 10954592 | C | 6.16 | 2.18E−02 |
| MACROD2 | 4813204 | G | 3.45 | 2.19E−02 |
| LOC100128590; | 11690292 | A | 3.43 | 2.22E−02 |
| SLC8A1 | | | | |
| KCNK2 | 4573492 | A | 3.30 | 2.35E−02 |
| NAV3 | 1479024 | C | 3.35 | 2.37E−02 |
| NLGN1 | 990634 | C | 4.20 | 2.37E−02 |
| CA10 | 1503056 | A | 3.20 | 2.39E−02 |
| OPCML | 7944972 | A | 3.49 | 2.45E−02 |
| MACROD2 | 16994969 | A | 3.94 | 2.46E−02 |
| MAGI2 | 2364341 | A | 5.37 | 2.48E−02 |
| PSD3 | 930023 | A | 5.05 | 2.49E−02 |
| MAGI2 | 6466510 | G | 3.84 | 2.50E−02 |
| NBEA | 1041304 | G | 4.11 | 2.51E−02 |
| CDH13 | 4783304 | C | 3.46 | 2.57E−02 |
| ABCA4 | 1801466 | A | 6.64 | 2.58E−02 |
| CADPS | 13067730 | C | 3.18 | 2.60E−02 |
| RYR2 | 7532996 | A | 3.78 | 2.60E−02 |
| MUSK | 16915435 | A | 4.31 | 2.64E−02 |
| NCEH1 | 4490388 | A | 3.62 | 2.65E−02 |
| KCNJ3 | 3111008 | G | 3.36 | 2.80E−02 |
| ATXN1 | 1399220 | A | 3.65 | 2.84E−02 |
| DGKB | 38273 | C | 4.16 | 2.84E−02 |
| SNTG1 | 2449960 | C | 7.09 | 2.86E−02 |
| RARB | 4393871 | C | 3.40 | 2.90E−02 |
| MUSK | 3001124 | C | 3.24 | 2.92E−02 |
| OPCML | 4937724 | A | 3.27 | 2.93E−02 |
| OPCML | 7110211 | G | 3.27 | 2.93E−02 |
| EPHA7 | 345741 | G | 5.39 | 2.93E−02 |
| NAV2 | 10766588 | A | 3.47 | 3.00E−02 |
| ATP2B2 | 4684689 | C | 2.89 | 3.07E−02 |
| NALCN | 1333761 | A | 3.14 | 3.13E−02 |
| DOK6 | 9965360 | A | 3.49 | 3.16E−02 |
| MYO3A | 3006260 | C | 8.20 | 3.17E−02 |
| IRF8 | 11647876 | A | 3.90 | 3.23E−02 |
| IRF8 | 9308366 | A | 3.90 | 3.23E−02 |
| RGS7 | 3912106 | C | 3.22 | 3.25E−02 |
| NFIL3 | 12683158 | C | 6.47 | 3.30E−02 |
| CSMD1 | 2720851 | G | 3.26 | 3.36E−02 |
| LOC100128590; | 404226 | A | 3.12 | 3.47E−02 |
| SLC8A1 | | | | |
| CNTN4 | 6787604 | A | 3.45 | 3.51E−02 |
| EPHB1 | 2138213 | C | 4.38 | 3.51E−02 |
| MAGI2 | 6949412 | A | 3.72 | 3.52E−02 |
| LOC100128590; | 11894296 | C | 3.84 | 3.52E−02 |
| SLC8A1 | | | | |
| EMID2 | 10953346 | C | 4.83 | 3.55E−02 |
| SEMA5A | 3026309 | A | 7.70 | 3.59E−02 |
| CLSTN2 | 4683773 | A | 3.61 | 3.60E−02 |
| OPCML | 12796925 | A | 5.44 | 3.62E−02 |
| NLGN1 | 10936778 | A | 3.67 | 3.63E−02 |
| MUSK | 10980573 | A | 4.19 | 3.65E−02 |
| NCAM2 | 2826692 | A | 3.47 | 3.68E−02 |
| NEDD4L | 12961713 | A | 3.84 | 3.68E−02 |
| FOXP1 | 831078 | A | 3.23 | 3.72E−02 |
| KIAA0182 | 11640338 | A | 3.21 | 3.75E−02 |
| TMEM132E | 998637 | C | 3.16 | 3.79E−02 |
| TMEM132E | 998638 | C | 3.16 | 3.79E−02 |
| CADPS | 6785029 | C | 3.22 | 3.80E−02 |
| KCNMA1 | 7921994 | C | 3.56 | 3.80E−02 |
| CA10 | 4794301 | A | 3.15 | 3.81E−02 |
| SELT | 6777911 | A | 3.24 | 3.83E−02 |
| NRCAM | 11561991 | A | 3.43 | 3.86E−02 |
| LRRK1 | 2924835 | A | 3.15 | 3.88E−02 |
| DLG2 | 1943687 | G | 3.55 | 3.94E−02 |
| PACRG | 9295202 | A | 3.20 | 4.01E−02 |
| MIR3974 | 1513050 | C | 3.68 | 4.03E−02 |
| FREM1 | 7041710 | C | 4.50 | 4.04E−02 |
| MDGA2 | 7160311 | C | 4.24 | 4.05E−02 |
| ADAMTS9; | 36115950 | A | 7.23 | 4.05E−02 |
| ADAMTS9-AS2 | | | | |
| MAP1B | 10062773 | A | 2.93 | 4.06E−02 |
| NRCAM | 10241406 | A | 3.78 | 4.07E−02 |
| CTNND2 | 2023916 | A | 2.87 | 4.09E−02 |
| PTPRT | 6030291 | C | 4.15 | 4.10E−02 |
| SEC23B | 6075350 | C | 3.51 | 4.11E−02 |
| PKP4 | 2193707 | C | 3.32 | 4.13E−02 |
| PDE4D | 6886495 | C | 4.69 | 4.14E−02 |
| C19orf45 | 475923 | A | 3.30 | 4.24E−02 |
| CNTN6 | 12490675 | C | 3.56 | 4.24E−02 |
| DOK6 | 8095385 | C | 3.18 | 4.27E−02 |
| MYO10 | 153708 | C | 3.19 | 4.28E−02 |
| RAB6B | 1104916 | A | 3.31 | 4.29E−02 |
| RAB6B | 940900 | A | 3.31 | 4.29E−02 |
| CTNND2 | 1494694 | A | 5.54 | 4.29E−02 |
| RYR3 | 1514033 | C | 2.91 | 4.30E−02 |
| RAB6B | 2692677 | C | 3.31 | 4.30E−02 |
| RIBC2 | 738227 | A | 3.08 | 4.31E−02 |
| ARNTL | 4757142 | A | 3.21 | 4.37E−02 |
| KIAA0182 | 754710 | C | 3.16 | 4.38E−02 |
| SH2D4B | 7097169 | A | 3.37 | 4.39E−02 |
| DOK6 | 4353548 | G | 3.41 | 4.46E−02 |
| RGS7 | 984402 | C | 3.27 | 4.47E−02 |
| INPP4A | 4851142 | A | 3.61 | 4.47E−02 |
| ROBO2 | 10514734 | C | 5.22 | 4.47E−02 |
| MAGI1 | 17073748 | A | 3.71 | 4.54E−02 |
| CNTNAP2 | 6464862 | A | 3.30 | 4.55E−02 |
| NPY | 16141 | C | 3.03 | 4.59E−02 |
| CCDC85A | 888279 | C | 3.27 | 4.68E−02 |
| NPY | 10951003 | A | 4.91 | 4.72E−02 |
| RAB6B | 940898 | A | 3.33 | 4.80E−02 |
| PSD3 | 13278489 | A | 5.41 | 4.81E−02 |
| TOX | 448650 | C | 3.40 | 4.90E−02 |
| DPP6 | 6961280 | A | 4.05 | 4.93E−02 |
| CACNG2 | 2283997 | C | 3.34 | 4.96E−02 |
| PLCB1 | 6118218 | A | 4.06 | 4.96E−02 |
| HAAO | 2241850 | A | 2.99 | 4.98E−02 |
| KCNB2 | 1107217 | A | 3.17 | 4.98E−02 |
| PREX2 | 4336596 | C | 3.19 | 4.99E−02 |

### Example 5

### Novel Haplotype-Tagging SNPs Impacting Response for Risperidone

Table 4A provides numerous examples of SNP alleles that predict good response to risperidone, and table 4B provides numerous examples of SNP alleles that predict poor response to risperidone. Tables 4A and 4B report the SNPs, SNP-alleles, P values, and Beta weights (in PANSS-Total units) from the linear regression for SNPs that affect response to risperidone. A negative beta weight indicates that the allele is associated with a decrease in PANSS-T score, corresponding to greater improvement (or lowering) of symptom burden. A positive beta weight indicates that the allele is associated with an increase in PANSS-T score, corresponding to a worsening (or increase) of symptom burden.

TABLE 4A
Alleles Influencing Good Response to Risperidone

| Gene | NCBI RS# | Allele | Beta(PANSS) | P |
|---|---|---|---|---|
| PSMD14 | 9713 | A | −6.27 | 4.99E−05 |
| LRP1B | 874295 | C | −6.60 | 1.85E−04 |
| TMEFF2 | 3738883 | C | −5.02 | 4.11E−04 |
| C7orf58 | 35793694 | A | −9.50 | 6.74E−04 |
| CAMK2D | 17620390 | A | −6.17 | 7.46E−04 |
| SLC35F3 | 12759054 | C | −5.63 | 7.83E−04 |
| CREB3L2 | 10954592 | C | −10.21 | 9.00E−04 |
| CACNB4 | 7597215 | C | −6.13 | 1.36E−03 |
| DNAH17 | 595711 | C | −5.79 | 1.45E−03 |
| SLC35F3 | 4641353 | A | −5.60 | 1.45E−03 |
| EXOC2 | 4409224 | A | −5.45 | 1.60E−03 |
| NRCAM | 1269658 | A | −4.63 | 2.09E−03 |
| MAMDC2; | 2148858 | C | −6.08 | 2.14E−03 |
| LOC100507299 | | | | |
| POLR2M | 11858659 | C | −9.29 | 2.50E−03 |
| KCNB2 | 349331 | C | −6.87 | 3.18E−03 |
| EPHA4 | 9758 | A | −4.65 | 3.55E−03 |
| NRXN3 | 17595443 | C | −9.47 | 3.58E−03 |
| DLEU2 | 2812200 | G | −4.76 | 3.59E−03 |
| PJA2 | 33730 | A | −4.41 | 4.80E−03 |
| KCNB2 | 4738266 | C | −4.60 | 4.91E−03 |
| ROBO1 | 35456279 | A | −8.86 | 5.08E−03 |
| CCDC50 | 35380043 | A | −12.06 | 5.10E−03 |
| LOC100129345 | 12435621 | G | −5.86 | 5.29E−03 |
| PLA2G2D | 578459 | A | −4.31 | 5.36E−03 |
| RIMBP2 | 12305517 | C | −6.36 | 5.46E−03 |
| PLCG2 | 11639517 | C | −5.59 | 5.63E−03 |
| CHRM3 | 7551001 | A | −4.04 | 5.65E−03 |
| ALK | 35093491 | A | −14.31 | 6.06E−03 |
| TMTC2 | 7974520 | A | −4.91 | 6.39E−03 |
| NALCN | 614728 | C | −4.30 | 7.04E−03 |
| CHRM3 | 12093821 | A | −3.83 | 8.04E−03 |
| PTPRN2 | 3952723 | A | −4.63 | 8.62E−03 |
| NBN | 1063054 | A | −4.49 | 9.00E−03 |
| EXOC2 | 4960043 | C | −4.17 | 9.12E−03 |
| IQGAP2 | 34950321 | C | −11.04 | 1.05E−02 |
| DTNBP1 | 3213207 | A | −6.69 | 1.05E−02 |
| DTNBP1 | 760761 | C | −5.39 | 1.05E−02 |
| DTNBP1 | 2619522 | G | −5.41 | 1.06E−02 |
| SERPINI1 | 2420034 | A | −3.96 | 1.06E−02 |
| CDH13 | 12600161 | C | −6.05 | 1.08E−02 |
| C14orf182 | 11626611 | C | −4.24 | 1.09E−02 |
| ATRNL1 | 1272383 | C | −7.28 | 1.12E−02 |
| PCSK5 | 10781342 | C | −4.83 | 1.16E−02 |
| NPAS3 | 7159875 | A | −4.19 | 1.18E−02 |
| PJA2 | 958976 | A | −3.84 | 1.20E−02 |
| ARPP21 | 2280096 | A | −3.72 | 1.20E−02 |
| NPAS3 | 10133174 | C | −4.15 | 1.22E−02 |
| CGNL1 | 6493933 | C | −3.82 | 1.26E−02 |
| ODZ3 | 2726789 | C | −4.79 | 1.31E−02 |
| PLA2G1B | 1179387 | A | −5.27 | 1.33E−02 |
| ITPR1 | 3805029 | C | −4.22 | 1.40E−02 |
| DGKB | 2116312 | C | −3.80 | 1.45E−02 |
| AKAP13 | 1808338 | C | −6.54 | 1.55E−02 |
| VPS41 | 2240555 | A | −3.96 | 1.55E−02 |
| CSMD1 | 688579 | A | −5.07 | 1.60E−02 |
| MYL12B | 894733 | A | −4.36 | 1.72E−02 |
| CSMD1 | 10503279 | C | −3.82 | 1.74E−02 |
| LOXL2 | 2280936 | C | −3.71 | 1.74E−02 |
| KAZN | 2004702 | A | −4.20 | 1.75E−02 |
| VTI1A | 3740144 | C | −7.41 | 1.77E−02 |
| SULT4A1 | 2071886 | A | −4.91 | 1.78E−02 |
| SULT4A1 | 4149442 | A | −4.91 | 1.78E−02 |
| CDH13 | 16958456 | A | −5.35 | 1.83E−02 |
| KCNB2 | 13264816 | C | −5.05 | 1.84E−02 |
| UNC5C | 10856915 | C | −3.57 | 1.84E−02 |
| ARPP21 | 2063648 | A | −3.55 | 1.84E−02 |
| NPY | 16141 | C | −3.83 | 1.88E−02 |
| PARD3B | 698911 | G | −4.84 | 1.90E−02 |
| EVC | 3774876 | C | −5.51 | 1.95E−02 |
| CSMD1 | 667595 | C | −4.83 | 1.98E−02 |
| EPHB2 | 11800828 | G | −3.48 | 2.01E−02 |
| BAALC | 17799604 | C | −4.95 | 2.02E−02 |
| ABT1 | 12204145 | A | −6.55 | 2.05E−02 |
| GDA | 1123 | C | −4.37 | 2.13E−02 |
| BAALC | 4734693 | C | −3.63 | 2.13E−02 |
| TMEFF2 | 13008804 | C | −3.56 | 2.13E−02 |
| LRRK1 | 2924835 | A | −3.86 | 2.17E−02 |
| LRP1B | 16847247 | A | −3.88 | 2.23E−02 |
| TMX2- | 12362406 | A | −4.74 | 2.24E−02 |
| CTNND1; | | | | |
| CTNND1 | | | | |
| COL22A1 | 10088210 | C | −3.66 | 2.30E−02 |
| NALCN | 583880 | A | −3.99 | 2.33E−02 |
| NALCN | 658213 | C | −3.99 | 2.33E−02 |
| PRKCE | 11125051 | G | −4.42 | 2.41E−02 |
| ST8SIA2 | 11853992 | A | −3.98 | 2.42E−02 |
| QRFP | 7034278 | C | −6.85 | 2.45E−02 |
| PARD3B | 1397482 | A | −3.65 | 2.46E−02 |
| CGNL1 | 766103 | G | −3.76 | 2.48E−02 |
| IP6K1 | 7634902 | T | −3.33 | 2.48E−02 |
| TBC1D22A | 15411 | C | −4.38 | 2.51E−02 |
| PSD3 | 13277215 | C | −3.20 | 2.58E−02 |
| SLC2A9 | 6817564 | A | −5.51 | 2.59E−02 |
| CHN2 | 3793259 | G | −3.76 | 2.62E−02 |
| SLC41A1 | 1772159 | A | −3.52 | 2.64E−02 |
| DOK6 | 10163684 | C | −4.80 | 2.66E−02 |
| PDE1C | 10228662 | A | −3.50 | 2.70E−02 |
| ANO2 | 7308729 | C | −3.68 | 2.71E−02 |
| DEAF1 | 4073590 | G | −3.31 | 2.72E−02 |
| TRIP 12 | 4973228 | G | −3.89 | 2.74E−02 |
| HS1BP3 | 35579164 | C | −9.52 | 2.79E−02 |
| PRKCE | 12619351 | G | −4.24 | 2.82E−02 |
| EXOC2 | 2473480 | A | −4.99 | 2.99E−02 |
| SLC2A13 | 4312128 | A | −4.47 | 3.07E−02 |
| DEAF1 | 17758 | A | −9.35 | 3.09E−02 |
| PPM1H | 3825305 | G | −6.45 | 3.09E−02 |
| SYNE1 | 2813539 | A | −3.29 | 3.15E−02 |
| ANO2 | 11063846 | C | −4.97 | 3.16E−02 |
| CNTN4 | 6787604 | A | −3.86 | 3.24E−02 |
| CAMKV | 6797500 | A | −7.59 | 3.25E−02 |
| ARPP21 | 4678793 | G | −3.21 | 3.26E−02 |
| CSMD1 | 2194358 | C | −3.77 | 3.28E−02 |
| FLJ45139 | 2836722 | A | −3.24 | 3.30E−02 |
| NPAS3 | 11156806 | A | −3.12 | 3.30E−02 |
| SORBS1 | 3740511 | C | −3.52 | 3.31E−02 |
| SLC2A13 | 10735885 | A | −4.39 | 3.37E−02 |
| SLC2A13 | 10784051 | C | −4.39 | 3.37E−02 |
| PLD5 | 4658813 | A | −3.14 | 3.45E−02 |
| LPHN3 | 1510924 | G | −3.81 | 3.46E−02 |
| TOX | 375878 | C | −4.48 | 3.52E−02 |
| ST8SIA2 | 2290492 | C | −3.95 | 3.56E−02 |
| FSTL5 | 1542071 | A | −5.10 | 3.57E−02 |
| NPY | 10951003 | A | −4.53 | 3.59E−02 |
| DOK6 | 12960929 | A | −3.31 | 3.63E−02 |
| NRG3 | 12415064 | A | −4.72 | 3.65E−02 |
| GALNTL4 | 11021757 | C | −4.11 | 3.68E−02 |
| IL17RD | 4299455 | A | −3.30 | 3.73E−02 |
| CDH8 | 11075447 | A | −3.50 | 3.76E−02 |
| ATP10A | 3816800 | C | −3.17 | 3.77E−02 |
| CSMD1 | 11136778 | C | −3.39 | 3.78E−02 |
| PLCB1 | 227131 | A | −3.24 | 3.83E−02 |
| RYR2 | 12135982 | C | −5.44 | 3.85E−02 |
| PCLO | 2715156 | A | −3.36 | 3.88E−02 |
| FBXL17 | 999063 | A | −4.69 | 3.90E−02 |
| UNC5C | 7681109 | A | −3.18 | 3.91E−02 |
| NPAS3 | 8014355 | A | −3.81 | 3.95E−02 |
| PTCHD4 | 9395327 | C | −3.26 | 3.95E−02 |
| OTOG | 7130190 | A | −5.31 | 4.04E−02 |
| NKAIN3 | 7388305 | C | −3.14 | 4.04E−02 |
| EPAS1 | 12614710 | G | −3.50 | 4.06E−02 |
| SVEP1 | 10980345 | C | −3.68 | 4.09E−02 |
| PCSK5 | 10869713 | C | −3.14 | 4.10E−02 |
| PTPRM | 565784 | A | −3.89 | 4.16E−02 |
| CACNA2D3 | 7617999 | A | −3.33 | 4.17E−02 |
| KCNIP1 | 870109 | A | −3.08 | 4.17E−02 |
| RGS7 | 6689169 | A | −4.69 | 4.21E−02 |
| RGS7 | 6700378 | A | −4.69 | 4.21E−02 |
| WWOX | 1125670 | C | −3.28 | 4.21E−02 |
| CSMD1 | 6995892 | A | −3.02 | 4.22E−02 |
| GPC6 | 10508001 | C | −3.78 | 4.23E−02 |
| GPC6 | 16948803 | C | −3.78 | 4.23E−02 |
| SLC35F3 | 6695594 | A | −3.54 | 4.25E−02 |
| CACNA2D3 | 10510770 | A | −3.73 | 4.26E−02 |
| UNC5C | 7679033 | C | −3.21 | 4.26E−02 |
| KCNN3 | 1218583 | A | −7.19 | 4.28E−02 |
| ANK3 | 10994154 | A | −4.88 | 4.28E−02 |
| CERKL | 2290517 | A | −4.66 | 4.33E−02 |
| DOK6 | 7228021 | G | −3.12 | 4.42E−02 |
| PDE10A | 220805 | A | −3.60 | 4.45E−02 |
| CAMKMT | 698826 | A | −4.03 | 4.48E−02 |
| RABGEF1 | 1060527 | C | −5.59 | 4.52E−02 |
| CLSTN2 | 1346134 | C | −3.97 | 4.53E−02 |
| DOK6 | 2886018 | C | −3.55 | 4.56E−02 |
| CSMD1 | 10108973 | A | −4.10 | 4.62E−02 |
| DGKB | 12666221 | G | −3.96 | 4.66E−02 |
| NKAIN2 | 1382648 | C | −5.84 | 4.67E−02 |
| GRIN3A | 10989597 | A | −3.73 | 4.69E−02 |
| LOC100505806 | 17329324 | C | −6.19 | 4.70E−02 |
| SORBS 1 | 7899506 | C | −3.30 | 4.75E−02 |
| MTSS1 | 10956192 | A | −4.22 | 4.77E−02 |
| MAML3 | 7690044 | G | −3.18 | 4.78E−02 |
| SGCZ | 17230584 | C | −4.87 | 4.80E−02 |
| MAGI2 | 12706063 | C | −3.24 | 4.80E−02 |
| KLF12 | 11841507 | C | −6.52 | 4.82E−02 |
| SVEP1 | 10816995 | C | −3.55 | 4.86E−02 |
| GPC5 | 9523734 | C | −2.98 | 4.89E−02 |
| EXOC2 | 17757367 | G | −4.59 | 4.92E−02 |
| PLCB1 | 1047383 | C | −3.05 | 4.93E−02 |
| PLCB1 | 6056229 | C | −3.05 | 4.93E−02 |
| RYR2 | 1521746 | C | −3.47 | 4.95E−02 |
| PRODH | 4819756 | A | −3.03 | 4.95E−02 |
| CCDC165 | 566890 | G | −4.34 | 4.99E−02 |
| GRID2 | 2904482 | A | −2.82 | 4.99E−02 |

TABLE 4B
Alleles Influencing Poor Response to Risperidone

| Gene | NCBI RS# | Allele | Beta(PANSS) | P |
|---|---|---|---|---|
| AGAP1 | 1869295 | C | 5.79 | 2.05E−04 |
| NPAS3 | 1315115 | C | 8.80 | 3.04E−04 |
| FER1L6; | 7840702 | A | 7.83 | 1.00E−03 |
| FER1L6-AS1 | | | | |
| FHIT | 6446117 | C | 5.37 | 1.71E−03 |
| DENND4C | 10122709 | C | 6.41 | 2.04E−03 |
| LRP1B | 355597 | C | 7.77 | 2.10E−03 |
| CTBP2 | 11245455 | C | 5.27 | 2.54E−03 |
| MAML3 | 2139926 | A | 4.69 | 3.23E−03 |
| CHRM3 | 4659552 | A | 4.43 | 3.99E−03 |
| LRRK1 | 1048327 | A | 6.21 | 4.45E−03 |
| NRG3 | 1039076 | C | 5.03 | 4.88E−03 |
| TBC1D22A | 9616150 | C | 6.35 | 5.11E−03 |
| LRP1B | 2290140 | C | 4.79 | 5.14E−03 |
| CBLB | 9832882 | A | 4.06 | 5.22E−03 |
| APBB2 | 4861314 | C | 4.33 | 5.69E−03 |
| DHODH | 2288000 | C | 4.31 | 5.92E−03 |
| RORA | 1965886 | C | 4.92 | 6.07E−03 |
| GBE1 | 2680277 | C | 4.22 | 6.17E−03 |
| GLDN | 2459395 | A | 4.98 | 6.26E−03 |
| SNX21; ACOT8 | 1057276 | A | 15.54 | 6.29E−03 |
| MSI2 | 8079426 | A | 4.37 | 6.75E−03 |
| BNC2 | 1999032 | C | 3.88 | 7.69E−03 |
| DENND4C | 2666797 | C | 4.86 | 7.90E−03 |
| NRXN3 | 8012563 | A | 5.86 | 8.52E−03 |
| CSMD1 | 17071342 | C | 6.03 | 8.86E−03 |
| CHN2 | 6965446 | A | 6.11 | 8.96E−03 |
| GAS7 | 17759453 | C | 4.07 | 9.28E−03 |
| SAG | 7565275 | A | 8.22 | 9.56E−03 |
| DLGAP1 | 498419 | A | 4.03 | 9.65E−03 |
| SORCS2 | 13442 | G | 3.99 | 9.71E−03 |
| TMX2-CTNND1 | 501738 | C | 4.62 | 1.01E−02 |
| GRB10 | 6967612 | C | 3.98 | 1.02E−02 |
| GRB10 | 7793570 | C | 3.98 | 1.02E−02 |
| GLP1R | 10305516 | C | 8.09 | 1.08E−02 |
| ATXN3 | 1047795 | C | 4.28 | 1.14E−02 |
| NCAM2 | 2826672 | C | 4.18 | 1.21E−02 |
| KIAA0947 | 1864117 | A | 3.87 | 1.23E−02 |
| WWOX | 2062894 | C | 3.97 | 1.23E−02 |
| SLC6A5 | 1443547 | A | 4.03 | 1.24E−02 |
| CGNL1 | 11071315 | A | 4.48 | 1.24E−02 |
| NXPH2 | 3732351 | A | 4.18 | 1.25E−02 |
| NPAS3 | 4982070 | C | 3.76 | 1.30E−02 |
| PDE4D | 16889901 | A | 4.11 | 1.32E−02 |
| SKAP1 | 2278868 | C | 3.90 | 1.34E−02 |
| WWOX | 9921980 | C | 4.43 | 1.39E−02 |
| ATF3 | 1126700 | A | 7.72 | 1.50E−02 |
| PCDH17 | 7319102 | A | 4.57 | 1.54E−02 |
| ABI2 | 11682759 | C | 5.06 | 1.55E−02 |
| ROBO1 | 17313129 | C | 5.96 | 1.58E−02 |
| PTPRG | 9844687 | A | 5.84 | 1.59E−02 |
| FLJ22447 | 698028 | C | 4.43 | 1.61E−02 |
| RYR2 | 12121792 | A | 3.96 | 1.63E−02 |
| PKIA | 2368508 | A | 3.76 | 1.65E−02 |
| ARPP21 | 9311104 | G | 3.61 | 1.67E−02 |
| PDE1C | 2041517 | C | 3.75 | 1.69E−02 |
| CBLB | 9288815 | G | 4.73 | 1.70E−02 |
| INMT-FAM188B; | 11972565 | A | 3.84 | 1.71E−02 |
| FAM188B | | | | |
| ITGA1 | 1047483 | A | 4.07 | 1.73E−02 |
| LRP1B | 13400449 | A | 4.02 | 1.75E−02 |
| KLHL29 | 17045819 | C | 5.51 | 1.75E−02 |
| ITGA1 | 6895049 | G | 4.07 | 1.78E−02 |
| LOC100506731 | 11159707 | C | 4.06 | 1.85E−02 |
| CBLB | 13073784 | C | 5.22 | 1.89E−02 |
| TBC1D1 | 2995920 | A | 3.62 | 1.91E−02 |
| MR1 | 16856699 | C | 5.08 | 1.95E−02 |
| CLASP2 | 4679034 | A | 5.85 | 2.06E−02 |
| GLDN | 2124874 | A | 4.45 | 2.11E−02 |
| PIKFYVE | 12622556 | A | 4.45 | 2.12E−02 |
| CCBE1 | 1791322 | C | 3.30 | 2.18E−02 |
| ARFGAP3 | 9607952 | C | 3.41 | 2.21E−02 |
| NPAS3 | 11622789 | T | 3.53 | 2.21E−02 |
| NOS1AP | 1504424 | C | 10.44 | 2.23E−02 |
| PLA2G4D | 776721 | C | 3.74 | 2.24E−02 |
| TMEM163 | 626501 | A | 4.47 | 2.26E−02 |
| NPAS3 | 17525387 | G | 6.05 | 2.28E−02 |
| RAB11FIP4 | 757375 | C | 3.52 | 2.30E−02 |
| PLEKHH2 | 919690 | C | 3.66 | 2.36E−02 |
| CNTNAP2 | 12667619 | A | 5.36 | 2.37E−02 |
| SAMD12 | 11562744 | A | 12.92 | 2.37E−02 |
| SAMD12 | 12540990 | A | 12.92 | 2.37E−02 |
| SAMD12 | 4302874 | A | 12.92 | 2.37E−02 |
| RORA | 4775308 | A | 4.81 | 2.38E−02 |
| ZNF365 | 2138564 | A | 3.62 | 2.39E−02 |
| MICAL2 | 11022209 | C | 7.35 | 2.40E−02 |
| CARD 11 | 6967255 | A | 2.73 | 2.42E−02 |
| GBE1 | 846 | C | 3.80 | 2.43E−02 |
| GRID2 | 6532374 | C | 4.01 | 2.53E−02 |
| PAPPA | 16933356 | C | 3.93 | 2.56E−02 |
| CGNL1 | 8027154 | C | 3.59 | 2.57E−02 |
| LOC286190; | 1979452 | C | 5.18 | 2.57E−02 |
| LACTB2 | | | | |
| NCAM2 | 2826671 | A | 3.81 | 2.62E−02 |
| SAAL1 | 951624 | A | 8.43 | 2.63E−02 |
| LOC728755 | 7149088 | A | 3.64 | 2.68E−02 |
| CSMD1 | 1442401 | G | 3.39 | 2.70E−02 |
| PTPRT | 6072649 | A | 4.82 | 2.75E−02 |
| MTSS1 | 8180920 | C | 3.50 | 2.78E−02 |
| LOC100289230 | 4703054 | A | 3.72 | 2.81E−02 |
| NTRK2 | 630426 | A | 4.11 | 2.84E−02 |
| CLSTN2 | 4450768 | A | 3.74 | 2.86E−02 |
| FAM69A | 2244496 | G | 6.41 | 2.87E−02 |
| BIRC6 | 2710625 | A | 3.25 | 2.93E−02 |
| EXOC4 | 17167240 | A | 3.69 | 2.95E−02 |
| ADAMTS19 | 10069990 | C | 6.48 | 3.00E−02 |
| ADAMTS19 | 1422472 | A | 6.48 | 3.00E−02 |
| ADAMTS19 | 1465686 | G | 6.48 | 3.00E−02 |
| CCDC165 | 7240959 | A | 3.32 | 3.02E−02 |
| DLC1 | 10888175 | C | 5.25 | 3.08E−02 |
| FSTL5 | 6826831 | C | 4.34 | 3.13E−02 |
| LRP1B | 10179688 | A | 4.51 | 3.13E−02 |
| KCNN3 | 4845677 | C | 3.49 | 3.18E−02 |
| ERBB4 | 1971801 | A | 3.49 | 3.18E−02 |
| NCAM2 | 2826668 | A | 3.68 | 3.24E−02 |
| CDH13 | 10871271 | A | 3.76 | 3.28E−02 |
| C15orf41 | 12708546 | A | 4.61 | 3.35E−02 |
| HHAT | 926581 | A | 3.37 | 3.37E−02 |
| ARL13B; STX19 | 13071953 | A | 5.76 | 3.37E−02 |
| FAM69A | 4240962 | A | 5.73 | 3.38E−02 |
| QPCT | 6708310 | A | 6.00 | 3.41E−02 |
| SYNE1 | 35591210 | C | 6.21 | 3.44E−02 |
| RORA | 8041466 | C | 3.38 | 3.46E−02 |
| NAV2 | 11819786 | A | 3.70 | 3.46E−02 |
| PRKG1 | 1937710 | C | 4.62 | 3.46E−02 |
| DFNB31 | 10982201 | A | 3.87 | 3.49E−02 |
| DFNB31 | 4979379 | C | 3.87 | 3.49E−02 |
| PDE1C | 11769133 | A | 4.10 | 3.50E−02 |
| RORA | 4775304 | A | 3.51 | 3.51E−02 |
| GLDN | 2168624 | C | 4.20 | 3.54E−02 |
| SNCA | 2301134 | T | 3.24 | 3.59E−02 |
| DGKB | 17168013 | A | 4.79 | 3.59E−02 |
| MACROD2 | 4813205 | A | 3.94 | 3.60E−02 |
| CAMKMT | 343954 | C | 4.19 | 3.65E−02 |
| KDM4C | 12379798 | C | 3.04 | 3.69E−02 |
| SGCZ | 12114757 | C | 3.82 | 3.79E−02 |
| NRG3 | 342368 | A | 3.62 | 3.82E−02 |
| DAB2IP | 12000723 | C | 3.79 | 3.82E−02 |
| LRP1B | 7558715 | A | 3.94 | 3.82E−02 |
| SLIT2 | 7663557 | A | 5.44 | 3.82E−02 |
| DOK6 | 12957360 | G | 3.11 | 3.95E−02 |
| CSMD1 | 2616996 | G | 3.49 | 3.97E−02 |
| HTR5A | 980442 | G | 9.32 | 3.97E−02 |
| OSBPL1A | 275861 | C | 3.06 | 4.01E−02 |
| SLIT1 | 4917756 | A | 4.36 | 4.01E−02 |
| CELF2 | 7068732 | G | 3.09 | 4.06E−02 |
| ABCA1 | 1800978 | C | 5.34 | 4.07E−02 |
| FRMD1 | 3823460 | C | 3.34 | 4.08E−02 |
| DGKB | 38292 | C | 4.10 | 4.14E−02 |
| CDH23 | 12260631 | A | 3.31 | 4.18E−02 |
| NCAM2 | 2826674 | A | 3.43 | 4.21E−02 |
| ATRNL1 | 12358411 | A | 3.54 | 4.23E−02 |
| SULT4A1 | 17570873 | C | 4.04 | 4.25E−02 |
| TMEM106B | 1042949 | C | 3.12 | 4.28E−02 |
| DGKB | 1859730 | C | 3.32 | 4.36E−02 |
| HS6ST3 | 2282135 | C | 3.72 | 4.37E−02 |
| PSD3 | 334749 | C | 6.95 | 4.38E−02 |
| MAGI2 | 12540925 | C | 3.39 | 4.39E−02 |
| DOK6 | 10871643 | A | 3.41 | 4.43E−02 |
| NAV3 | 2030897 | C | 3.62 | 4.45E−02 |
| ATP2B2 | 4327369 | C | 3.36 | 4.49E−02 |
| INMT-FAM188B; | 10230286 | A | 3.19 | 4.63E−02 |
| FAM188B | | | | |
| CLASP2 | 12492003 | A | 4.90 | 4.66E−02 |
| LRP1B | 970600 | A | 3.95 | 4.75E−02 |
| THBS4 | 3813667 | C | 2.89 | 4.77E−02 |
| NALCN | 9557591 | C | 3.90 | 4.81E−02 |
| LOC100506128 | 10913414 | A | 4.60 | 4.82E−02 |
| KCNK10 | 10151231 | A | 5.04 | 4.82E−02 |
| SORCS3 | 7068684 | C | 4.15 | 4.84E−02 |
| CDH13 | 3935908 | G | 3.22 | 4.91E−02 |
| NTM | 487518 | C | 3.19 | 4.92E−02 |
| VPS41 | 1061303 | C | 5.74 | 4.93E−02 |
| PPM1H | 17732506 | G | 6.16 | 4.94E−02 |
| NRG3 | 2820111 | A | 3.44 | 4.95E−02 |
| DPP6 | 10952464 | A | 4.13 | 4.95E−02 |
| GRB10 | 1019000 | C | 3.16 | 4.97E−02 |

### Example 6

### Novel Haplotype-Tagging SNPs Impacting Response for Ziprasidone

Table 5A provides numerous examples of SNP alleles that predict good response to ziprasidone, and table 5B provides numerous examples of SNP alleles that predict poor response to ziprasidone. Tables 5A and 5B report the SNPs, SNP-alleles, P values, and Beta weights (in PANSS-Total units) from the linear regression for SNPs that affect response to ziprasidone. A negative beta weight indicates that the allele is associated with a decrease in PANSS-T score, corresponding to greater improvement (or lowering) of symptom burden. A positive beta weight indicates that the allele is associated with an increase in PANSS-T score, corresponding to a worsening (or increase) of symptom burden.

TABLE 5A
Alleles Influencing Good Response to Ziprasidone

| Gene | NCBI RS# | Allele | Beta(PANSS) | P |
|---|---|---|---|---|
| CDH4 | 4925300 | A | −7.60 | 6.29E−05 |
| LYN | 1546519 | C | −6.40 | 1.31E−04 |
| CNTN4 | 17194378 | A | −5.87 | 4.41E−04 |
| CDH4 | 4925199 | G | −6.52 | 1.06E−03 |
| KITLG | 995029 | C | −7.41 | 1.38E−03 |
| ARHGAP31 | 12495539 | C | −6.37 | 1.76E−03 |
| HIATL1 | 9409550 | C | −4.98 | 1.92E−03 |
| LOC100616530 | 2319150 | G | −4.97 | 2.27E−03 |
| BAG3 | 35434411 | C | −11.89 | 2.37E−03 |
| NALCN | 7993937 | A | −6.10 | 2.63E−03 |
| FHIT | 17670088 | A | −5.84 | 3.47E−03 |
| ODZ2 | 17526010 | C | −5.89 | 3.90E−03 |
| SGCZ | 1454583 | C | −5.86 | 4.05E−03 |
| ODZ2 | 11134473 | A | −5.98 | 4.36E−03 |
| SGCZ | 2199910 | A | −5.30 | 4.87E−03 |
| NEDD9 | 17495074 | C | −6.31 | 4.92E−03 |
| ZNF169 | 7042481 | A | −4.62 | 5.08E−03 |
| ANK2 | 34270799 | A | −12.77 | 5.33E−03 |
| C15orf41 | 2111015 | G | −5.95 | 6.03E−03 |
| SGCZ | 2168123 | C | −5.61 | 6.35E−03 |
| CAPZB, | 10799809 | C | −4.47 | 6.74E−03 |
| LOC644083 | | | | |
| CSMD1 | 1531532 | C | −4.55 | 6.77E−03 |
| KCNB2 | 7465440 | A | −6.79 | 6.97E−03 |
| ADAMTSL1 | 10811035 | A | −4.68 | 7.11E−03 |
| TMEM181 | 9364984 | A | −4.91 | 7.12E−03 |
| METTL21A | 4234080 | A | −6.10 | 7.23E−03 |
| PTPRG | 12635719 | A | −6.64 | 7.71E−03 |
| ATP10A | 4609818 | A | −5.44 | 7.80E−03 |
| SORCS3 | 2496017 | A | −5.64 | 7.82E−03 |
| NPAS3 | 12100765 | C | −4.85 | 7.84E−03 |
| ANK3 | 12354956 | A | −4.96 | 8.73E−03 |
| LRP1B | 13400449 | A | −5.21 | 1.05E−02 |
| VSNL1 | 2680832 | A | −4.17 | 1.05E−02 |
| LOXL2 | 2280936 | C | −4.02 | 1.05E−02 |
| RGS7 | 2815849 | C | −6.56 | 1.08E−02 |
| SYCP2 | 13039338 | G | −14.84 | 1.12E−02 |
| MAGI1 | 13086093 | C | −4.83 | 1.14E−02 |
| NALCN | 1452113 | G | −5.41 | 1.16E−02 |
| TSPAN9 | 537938 | A | −4.43 | 1.18E−02 |
| RAB36 | 5751596 | A | −4.38 | 1.19E−02 |
| SCUBE1 | 5759274 | A | −3.96 | 1.19E−02 |
| DYNC1I1 | 916758 | C | −5.76 | 1.21E−02 |
| RC3H1 | 2103640 | A | −4.75 | 1.21E−02 |
| NXPH1 | 17150874 | C | −7.08 | 1.22E−02 |
| SLC1A3 | 891189 | G | −4.27 | 1.23E−02 |
| GLP1R | 1820 | A | −9.85 | 1.28E−02 |
| DFNB31 | 4979379 | C | −5.48 | 1.28E−02 |
| CNTN4 | 908490 | A | −4.24 | 1.29E−02 |
| CSMD1 | 12549644 | A | −5.91 | 1.30E−02 |
| CDH7 | 974080 | C | −5.07 | 1.34E−02 |
| CDH13 | 17701213 | C | −7.08 | 1.38E−02 |
| HYDIN | 1774416 | C | −6.82 | 1.38E−02 |
| EYA4 | 6569879 | A | −4.60 | 1.38E−02 |
| SHC3 | 10122011 | C | −4.18 | 1.45E−02 |
| PES1 | 42942 | A | −7.23 | 1.46E−02 |
| PRKG1 | 10997954 | C | −4.51 | 1.50E−02 |
| ADAMTS19 | 4836459 | A | −5.20 | 1.55E−02 |
| PDE1C | 11769133 | A | −6.03 | 1.58E−02 |
| HAAO | 3755540 | C | −4.79 | 1.61E−02 |
| CTNNA3, | 7902006 | C | −6.58 | 1.62E−02 |
| LRRTM3 | | | | |
| CAPZB, | 6698682 | G | −4.02 | 1.67E−02 |
| LOC644083 | | | | |
| ODZ2 | 1421991 | A | −8.64 | 1.70E−02 |
| ADAMTSL1 | 4977432 | C | −6.16 | 1.70E−02 |
| MTSS1 | 891541 | C | −4.72 | 1.71E−02 |
| LIMCH1 | 1377349 | G | −4.89 | 1.72E−02 |
| SRRM4 | 1568923 | C | −4.43 | 1.74E−02 |
| NRXN3 | 2199796 | A | −7.27 | 1.75E−02 |
| NALCN | 1289556 | G | −4.33 | 1.75E−02 |
| QRFPR | 11098616 | A | −4.20 | 1.80E−02 |
| SDK1 | 17133636 | A | −7.23 | 1.81E−02 |
| MAGI2 | 7789112 | A | −3.86 | 1.81E−02 |
| SGCZ | 17574120 | G | −3.93 | 1.83E−02 |
| SDK1 | 10807838 | C | −3.75 | 1.86E−02 |
| ODZ2 | 4868807 | C | −6.30 | 1.87E−02 |
| PLCG2 | 7197832 | A | −4.46 | 1.91E−02 |
| NCKAP5 | 281580 | A | −4.17 | 1.91E−02 |
| FGF5 | 3733336 | A | −4.10 | 1.92E−02 |
| ADAMTSL1 | 10811036 | C | −5.69 | 1.95E−02 |
| INPP4A | 12988976 | A | −8.49 | 1.96E−02 |
| TSPAN11 | 3741868 | A | −9.20 | 2.04E−02 |
| GABRP | 11745599 | A | −4.76 | 2.06E−02 |
| FBXL17 | 288173 | C | −5.30 | 2.10E−02 |
| GRM8 | 17865092 | C | −8.62 | 2.12E−02 |
| F5 | 6018 | G | −9.77 | 2.16E−02 |
| MICAL2 | 11022209 | C | −6.38 | 2.16E−02 |
| MAML3 | 7690044 | G | −3.98 | 2.17E−02 |
| CDH7 | 2291343 | A | −4.73 | 2.19E−02 |
| FBXL17 | 288180 | C | −5.29 | 2.22E−02 |
| GABBR2 | 2304391 | A | −6.08 | 2.23E−02 |
| SHC3 | 944478 | T | −3.75 | 2.25E−02 |
| PLD5 | 2809995 | A | −3.66 | 2.25E−02 |
| KLHL29 | 934373 | A | −4.12 | 2.26E−02 |
| DOK6 | 1790602 | C | −7.75 | 2.28E−02 |
| MSI2 | 17834337 | A | −7.74 | 2.30E−02 |
| ODZ2 | 1421988 | G | −3.89 | 2.31E−02 |
| ODZ2 | 1421978 | C | −3.77 | 2.34E−02 |
| QRFPR | 11737010 | A | −4.04 | 2.38E−02 |
| LIMCH1 | 1377348 | C | −4.63 | 2.44E−02 |
| KLHL29 | 1709304 | A | −3.63 | 2.51E−02 |
| ODZ2 | 6879227 | A | −4.60 | 2.56E−02 |
| ODZ2 | 2973664 | A | −4.60 | 2.56E−02 |
| RYR3 | 16970951 | A | −4.41 | 2.56E−02 |
| CDH13 | 17768659 | C | −5.59 | 2.58E−02 |
| LOC100289130, | 1800668 | C | −4.24 | 2.61E−02 |
| GPX1 | | | | |
| LIMCH1 | 1453043 | C | −4.24 | 2.62E−02 |
| DGKD | 838718 | A | −3.93 | 2.62E−02 |
| PARK2 | 9458300 | A | −4.78 | 2.66E−02 |
| SCD5 | 7657237 | C | −5.76 | 2.69E−02 |
| CDS1 | 1372971 | A | −3.82 | 2.72E−02 |
| ATF6 | 6427628 | A | −6.77 | 2.73E−02 |
| RORA | 340005 | A | −3.68 | 2.73E−02 |
| TMEM106B | 1042949 | C | −3.36 | 2.81E−02 |
| KCNJ3 | 2591172 | G | −3.86 | 2.86E−02 |
| CDH13 | 8053315 | G | −4.37 | 2.93E−02 |
| CDH7 | 12607785 | A | −4.48 | 2.95E−02 |
| CDH7 | 4580293 | C | −4.48 | 2.95E−02 |
| NCAM2 | 6518020 | G | −3.47 | 2.96E−02 |
| PRUNE2 | 620552 | C | −4.74 | 2.97E−02 |
| PRUNE2 | 512110 | C | −4.74 | 2.97E−02 |
| DGKD | 838717 | A | −3.87 | 2.97E−02 |
| CPLX2 | 3749801 | C | −8.65 | 3.00E−02 |
| C15orf41 | 2381887 | A | −4.31 | 3.01E−02 |
| MAGI1 | 2306379 | C | −4.50 | 3.02E−02 |
| SGCZ | 1454580 | C | −3.75 | 3.02E−02 |
| FHIT | 2736792 | A | −3.87 | 3.03E−02 |
| TRPC4 | 3812841 | A | −3.71 | 3.05E−02 |
| OPCML | 11223232 | A | −3.85 | 3.06E−02 |
| GIGYF2 | 2289915 | A | −4.58 | 3.10E−02 |
| PTPRG | 11721138 | C | −5.79 | 3.15E−02 |
| PTPRG | 9832251 | C | −5.79 | 3.15E−02 |
| SPIB | 1137895 | C | −4.27 | 3.17E−02 |
| CDH23 | 7902757 | C | −9.95 | 3.18E−02 |
| NELL1 | 12223203 | A | −4.29 | 3.18E−02 |
| VSNL1 | 1996610 | C | −3.56 | 3.19E−02 |
| NCKAP5 | 12999715 | A | −4.14 | 3.24E−02 |
| NRXN3 | 10146690 | A | −4.73 | 3.28E−02 |
| NRXN3 | 12147298 | A | −5.05 | 3.30E−02 |
| DENND5B | 3741876 | A | −5.69 | 3.34E−02 |
| USH2A | 11117573 | A | −4.59 | 3.35E−02 |
| NLGN1 | 3980098 | G | −3.61 | 3.38E−02 |
| MAGI2 | 6949538 | A | −5.38 | 3.44E−02 |
| BMP7 | 17480735 | A | −6.71 | 3.51E−02 |
| PCLO | 17210284 | C | −3.87 | 3.56E−02 |
| CDH4 | 6142887 | C | −12.39 | 3.57E−02 |
| GALNTL4 | 10741551 | C | −4.13 | 3.58E−02 |
| CACNB2 | 6482385 | C | −3.79 | 3.58E−02 |
| SH2D4B | 7097169 | A | −4.18 | 3.62E−02 |
| KIAA0182 | 11640338 | A | −3.84 | 3.70E−02 |
| SDK1 | 4722654 | A | −3.38 | 3.71E−02 |
| CDH13 | 7185276 | A | −4.77 | 3.90E−02 |
| LRRC4C | 10768581 | C | −3.50 | 3.98E−02 |
| NRXN3 | 2192426 | G | −3.26 | 3.98E−02 |
| WWOX | 9921059 | G | −3.54 | 4.04E−02 |
| SH3GL3 | 2730082 | A | −3.92 | 4.08E−02 |
| ABCA13 | 11983883 | G | −5.63 | 4.10E−02 |
| TBXAS1 | 193948 | G | −3.59 | 4.14E−02 |
| RYR2 | 4659797 | A | −6.26 | 4.16E−02 |
| ARHGAP31 | 751607 | A | −5.36 | 4.23E−02 |
| SLC22A23 | 9392478 | C | −3.36 | 4.23E−02 |
| DOCK1 | 2229603 | A | −20.43 | 4.26E−02 |
| ADCY8 | 913818 | A | −6.73 | 4.28E−02 |
| CTBP2 | 2938009 | G | −3.60 | 4.37E−02 |
| PARK2 | 9295174 | C | −4.36 | 4.42E−02 |
| DLC1 | 2027 | A | −10.36 | 4.43E−02 |
| MAGI1 | 2372067 | A | −3.56 | 4.44E−02 |
| CACNA1E | 638132 | A | −3.71 | 4.45E−02 |
| COL6A3 | 4663722 | C | −6.40 | 4.49E−02 |
| SNRNP27 | 1048139 | A | −4.89 | 4.49E−02 |
| PTPRT | 1569549 | A | −5.49 | 4.50E−02 |
| FBXL2 | 12330707 | A | −3.98 | 4.50E−02 |
| COL6A3 | 36117715 | A | −8.54 | 4.58E−02 |
| LIMCH1 | 11735207 | C | −3.72 | 4.63E−02 |
| RORA | 8037420 | C | −3.55 | 4.64E−02 |
| PDE1C | 10951313 | C | −3.25 | 4.68E−02 |
| MACROD2 | 1013112 | A | −3.35 | 4.73E−02 |
| RGS7 | 2678780 | A | −4.17 | 4.78E−02 |
| GAS7 | 7216101 | A | −5.42 | 4.79E−02 |
| PCDH17 | 9537776 | A | −3.46 | 4.80E−02 |
| STK10 | 15963 | C | −4.48 | 4.84E−02 |
| GRK5 | 4623810 | G | −3.45 | 4.85E−02 |
| KCNJ3 | 3106660 | C | −10.17 | 4.86E−02 |
| CELSR3 | 6773261 | A | −5.90 | 4.87E−02 |
| DFNB31 | 10982201 | A | −4.35 | 4.88E−02 |
| TSPAN5 | 10020677 | A | −3.50 | 4.88E−02 |
| TMEM132B | 2240497 | A | −3.32 | 4.89E−02 |
| LRRC4C | 4375425 | A | −3.39 | 4.90E−02 |
| SHROOM3 | 12710873 | A | −4.25 | 4.95E−02 |
| CELSR3 | 3821875 | C | −6.04 | 4.97E−02 |

TABLE 5B
Alleles Influencing a Poor Response to Ziprasidone

| Gene | NCBI RS# | Allele | Beta(PANSS) | P |
|---|---|---|---|---|
| BMPR1B | 17023107 | C | 23.68 | 2.51E−05 |
| UNC5C | 17023119 | C | 23.68 | 2.51E−05 |
| NALCN | 9585618 | C | 5.77 | 4.79E−04 |
| SLIT2 | 12233652 | A | 6.24 | 7.97E−04 |
| BLZF1 | 2275299 | C | 5.37 | 1.00E−03 |
| NBAS | 12692258 | A | 6.91 | 1.05E−03 |
| NALCN | 651737 | C | 5.83 | 1.30E−03 |
| NALCN | 614728 | C | 5.38 | 1.59E−03 |
| GRIA1 | 10036589 | C | 7.42 | 1.76E−03 |
| NALCN | 583880 | A | 5.65 | 1.77E−03 |
| NALCN | 658213 | C | 5.65 | 1.77E−03 |
| PSD3 | 335244 | C | 5.20 | 2.03E−03 |
| MACROD2 | 13043406 | C | 10.71 | 2.24E−03 |
| MUSK | 16915435 | A | 6.63 | 3.26E−03 |
| MUSK | 10980573 | A | 6.94 | 3.58E−03 |
| ITGA1 | 1047483 | T | 5.06 | 4.25E−03 |
| ITGA1 | 6895049 | T | 5.06 | 4.25E−03 |
| TRPM3 | 10780982 | G | 4.73 | 4.53E−03 |
| PARD3B | 10490272 | A | 5.76 | 4.69E−03 |
| FMN2 | 6699880 | A | 5.71 | 5.05E−03 |
| PARD3B | 724605 | A | 5.16 | 5.22E−03 |
| CDH4 | 6061338 | C | 5.64 | 5.68E−03 |
| WWC1 | 10042345 | C | 4.82 | 5.98E−03 |
| S100PBP | 1284365 | C | 5.91 | 6.05E−03 |
| BCL2L11 | 6753785 | G | 4.22 | 6.28E−03 |
| VSNL1 | 7593881 | C | 4.68 | 6.47E−03 |
| PTPRG | 12495140 | T | 4.56 | 6.51E−03 |
| CDS1 | 1120 | C | 5.55 | 6.60E−03 |
| PARD3B | 12613874 | A | 4.30 | 6.81E−03 |
| EPHB1 | 1004551 | A | 4.85 | 7.80E−03 |
| NALCN | 9557581 | C | 4.45 | 7.87E−03 |
| APBB2 | 4861314 | C | 4.65 | 9.10E−03 |
| EPHB1 | 1980139 | G | 6.04 | 9.18E−03 |
| DAPK1 | 1007394 | C | 3.89 | 9.22E−03 |
| SLC25A21 | 17105059 | C | 5.72 | 9.44E−03 |
| DAPK1 | 3118854 | C | 3.93 | 9.71E−03 |
| TMEFF2 | 13008804 | C | 4.18 | 1.01E−02 |
| CNTNAP2 | 2692132 | A | 4.56 | 1.05E−02 |
| KCNIP1 | 50057 | A | 4.83 | 1.12E−02 |
| CTNNA2 | 2862025 | C | 5.49 | 1.13E−02 |
| TRPM3 | 10511991 | A | 4.80 | 1.15E−02 |
| GRID2 | 992995 | A | 4.30 | 1.18E−02 |
| ARNTL | 4757138 | A | 4.61 | 1.19E−02 |
| ARNTL | 2279287 | A | 4.61 | 1.19E−02 |
| ARNTL | 2279286 | C | 4.61 | 1.19E−02 |
| ARNTL | 2279285 | A | 4.61 | 1.19E−02 |
| ARNTL | 2279284 | A | 4.61 | 1.19E−02 |
| ERC2 | 187205 | C | 4.56 | 1.27E−02 |
| SYNE1 | 1408461 | A | 4.28 | 1.29E−02 |
| ARNTL | 10766075 | C | 4.40 | 1.32E−02 |
| RYR2 | 10925398 | A | 4.43 | 1.36E−02 |
| RHOG | 4597058 | C | 4.73 | 1.45E−02 |
| DPYSL5 | 1371614 | C | 4.32 | 1.47E−02 |
| DLGAP1 | 11081059 | A | 4.61 | 1.50E−02 |
| NKAIN2 | 9372762 | A | 4.09 | 1.51E−02 |
| DLGAP1 | 1791398 | A | 4.60 | 1.51E−02 |
| NBEA | 9600401 | C | 7.63 | 1.55E−02 |
| UTRN | 12204734 | A | 5.75 | 1.56E−02 |
| EXOC2 | 17755910 | C | 6.16 | 1.61E−02 |
| COL4A4 | 1320407 | G | 4.07 | 1.62E−02 |
| ZNF169 | 10993153 | C | 6.00 | 1.65E−02 |
| CACNA2D1 | 37090 | A | 3.78 | 1.74E−02 |
| DAPK1 | 3128480 | C | 3.49 | 1.76E−02 |
| PACRG | 1333955 | G | 3.72 | 1.76E−02 |
| UTRN | 9321977 | G | 4.91 | 1.76E−02 |
| PRKCE | 7601378 | A | 4.37 | 1.77E−02 |
| PKP4 | 2193707 | C | 4.55 | 1.78E−02 |
| KCNMA1 | 6480850 | C | 3.76 | 1.79E−02 |
| SEMA3E | 2723017 | A | 5.51 | 1.85E−02 |
| DLG2 | 921452 | A | 6.03 | 1.89E−02 |
| GPC5 | 9523734 | C | 3.59 | 2.03E−02 |
| DGKI | 2278829 | A | 4.10 | 2.03E−02 |
| TBC1D1 | 12643286 | A | 4.58 | 2.04E−02 |
| FGF14 | 2390674 | A | 5.74 | 2.08E−02 |
| PTPRN2 | 3952723 | A | 4.52 | 2.20E−02 |
| ULK1 | 10902469 | C | 10.56 | 2.24E−02 |
| KCNMA1 | 1907727 | C | 4.82 | 2.31E−02 |
| PLA2R1 | 3109389 | C | 5.49 | 2.33E−02 |
| SLC35F3 | 10157061 | T | 3.93 | 2.34E−02 |
| CNTN5 | 10790978 | G | 4.47 | 2.35E−02 |
| UTRN | 10457761 | C | 4.87 | 2.43E−02 |
| CCDC50 | 35380043 | A | 9.56 | 2.47E−02 |
| INSC | 17507577 | A | 6.31 | 2.49E−02 |
| SDK1 | 10233166 | A | 3.63 | 2.52E−02 |
| KCNK9 | 2542425 | A | 3.93 | 2.52E−02 |
| CDH13 | 7186797 | C | 3.56 | 2.54E−02 |
| CTNNA2 | 12713991 | G | 3.89 | 2.61E−02 |
| TMEFF2 | 3738883 | C | 3.72 | 2.62E−02 |
| EXOC2 | 17757367 | G | 5.83 | 2.65E−02 |
| RAB11FIP4 | 1076185 | G | 4.77 | 2.70E−02 |
| PDE4D | 6450528 | G | 4.12 | 2.71E−02 |
| FREM1 | 16932300 | C | 8.78 | 2.71E−02 |
| RARB | 871963 | A | 3.48 | 2.77E−02 |
| TPH2 | 1386493 | C | 4.05 | 2.80E−02 |
| ATP10A | 12050652 | C | 3.36 | 2.81E−02 |
| MICAL2 | 11022220 | A | 4.96 | 2.81E−02 |
| LRP1B | 10193058 | A | 3.88 | 2.83E−02 |
| CDH13 | 17289333 | G | 4.62 | 2.88E−02 |
| C12orf5 | 1046165 | C | 4.84 | 2.88E−02 |
| SYT13 | 3816205 | A | 5.87 | 2.90E−02 |
| ARHGAP19-SLIT1 | 3758587 | C | 4.32 | 2.92E−02 |
| DPYSL5 | 10181727 | C | 3.46 | 2.97E−02 |
| DPYSL5 | 1057115 | C | 3.46 | 2.97E−02 |
| CTNND2 | 6879413 | C | 4.80 | 3.02E−02 |
| CCBE1 | 1791322 | C | 3.49 | 3.03E−02 |
| CDH13 | 8182105 | C | 5.35 | 3.04E−02 |
| FSTL5 | 13141680 | C | 4.27 | 3.07E−02 |
| DLG2 | 7109065 | C | 5.32 | 3.11E−02 |
| SYT13 | 7395419 | A | 4.47 | 3.12E−02 |
| SYT13 | 7395421 | A | 4.47 | 3.12E−02 |
| UBL3 | 9578136 | C | 4.30 | 3.13E−02 |
| UBL3 | 957189 | C | 4.30 | 3.13E−02 |
| CTBP2 | 3781412 | C | 3.59 | 3.22E−02 |
| SAMD4A | 1211170 | A | 3.78 | 3.25E−02 |
| NAV3 | 964639 | A | 3.43 | 3.27E−02 |
| KYNU | 10496935 | C | 3.80 | 3.31E−02 |
| CACNA2D3 | 1851043 | C | 3.78 | 3.35E−02 |
| PSD3 | 335221 | C | 3.76 | 3.36E−02 |
| FMN2 | 7530215 | G | 4.40 | 3.38E−02 |
| CSMD1 | 13271457 | A | 3.73 | 3.40E−02 |
| KIAA1797 | 9886755 | C | 4.95 | 3.51E−02 |
| NRXN3 | 8017544 | C | 3.61 | 3.71E−02 |
| NCKAP5 | 16841277 | A | 4.16 | 3.82E−02 |
| F5 | 2187952 | A | 3.71 | 3.89E−02 |
| CHN2 | 6965446 | A | 5.17 | 3.95E−02 |
| ROBO1 | 9855098 | A | 4.32 | 3.96E−02 |
| MBP | 6565924 | A | 3.70 | 3.99E−02 |
| MICAL2 | 12274943 | C | 3.87 | 4.03E−02 |
| MIER1 | 6681625 | A | 14.71 | 4.04E−02 |
| PTGS2 | 20417 | C | 5.28 | 4.05E−02 |
| MAGI2 | 2885559 | C | 3.54 | 4.07E−02 |
| CSMD1 | 7813376 | C | 3.88 | 4.18E−02 |
| DLG2 | 7937832 | G | 3.88 | 4.20E−02 |
| COL22A1 | 7000416 | C | 3.71 | 4.25E−02 |
| CDH23 | 10999803 | C | 6.66 | 4.27E−02 |
| MIR1270-1 | 7247683 | C | 3.68 | 4.29E−02 |
| AKAP6 | 4647899 | A | 4.17 | 4.32E−02 |
| ARHGEF10 | 14375 | G | 4.92 | 4.33E−02 |
| TPH2 | 1872824 | C | 3.47 | 4.35E−02 |
| SLCO3A1 | 4312282 | A | 4.01 | 4.37E−02 |
| CCDC88C | 7146512 | C | 3.50 | 4.39E−02 |
| MTO1 | 1713862 | C | 6.02 | 4.41E−02 |
| CNTN4 | 13086027 | A | 4.04 | 4.44E−02 |
| DOK6 | 9989625 | C | 3.48 | 4.45E−02 |
| SLC1A3 | 10491374 | G | 3.56 | 4.48E−02 |
| C10orf112 | 10763975 | A | 5.05 | 4.49E−02 |
| GRIN3A | 4324970 | C | 3.28 | 4.50E−02 |
| FHIT | 492836 | T | 3.39 | 4.50E−02 |
| WWOX | 13335579 | C | 3.41 | 4.50E−02 |
| MBP | 4890788 | C | 3.49 | 4.50E−02 |
| PCSK6 | 4965881 | C | 3.99 | 4.51E−02 |
| PCSK6 | 7166590 | A | 3.99 | 4.51E−02 |
| FMN2 | 994277 | A | 3.35 | 4.52E−02 |
| TPH2 | 1386482 | A | 3.37 | 4.57E−02 |
| TRDN | 11759636 | A | 4.15 | 4.64E−02 |
| CNTNAP2 | 6964783 | C | 3.45 | 4.65E−02 |
| CRISPLD2 | 16974880 | G | 3.69 | 4.65E−02 |
| MMP16 | 10103111 | C | 4.39 | 4.66E−02 |
| CTNND2 | 6859430 | C | 6.30 | 4.71E−02 |
| MIOX | 1055271 | C | 3.47 | 4.72E−02 |
| PTPRN2 | 6978278 | A | 3.79 | 4.77E−02 |
| COL4A4 | 12468501 | A | 4.32 | 4.79E−02 |
| TMC8 | 11655790 | C | 8.46 | 4.79E−02 |
| TRPM6 | 9650767 | A | 3.55 | 4.81E−02 |
| ZXDC | 7131 | C | 3.61 | 4.81E−02 |
| GRID2 | 10004009 | A | 3.45 | 4.88E−02 |
| SH3GL3 | 1491578 | C | 4.27 | 4.89E−02 |
| NAALADL2 | 7640306 | A | 3.97 | 4.95E−02 |
| LRP1B | 13398962 | C | 4.86 | 5.00E−02 |

### Example 7

### Identification of SNPs Tagging the Same Haplotypes

The inventors determined which of the various SNPs that impact antipsychotic response, correlate with one another indicating that they, in fact, tag the same haplotype. To determine which of the various SNP genotypes for SNPs in Tables 1-5 correlate with one another, the inventors calculated the pair-wise correlation coefficient using cor function in R (version 2.15.1). This was done for each antipsychotic drug separately so that correlation coefficients were calculated for pairs of SNPs within Table 1, within Table 2, within Table 3, within Table 4, and within Table 5. Two SNPs were considered correlated if their Pearson correlation coefficient r≧0.8 (or r²≧0.64). Note that this approach identifies SNPs that are redundant in terms of tagging haplotypes in that SNPs with correlated genotypes, by definition, tag the same haplotype. The inventors define such SNPs as being members of the same “correlating cluster”.

Tables 6 to 10 show SNPs that both impact the response to the same antipsychotic medication, and that are members of the same correlating cluster, thus tagging the same haplotype. Table 6 shows correlating clusters for olanzapine, Table 7 for perphenazine, Table 8 for quetiapine, Table 9 for risperidone, and Table 10 for ziprasidone. Any SNP from Tables 1-5 that is not listed in the corresponding Table 6-10 uniquely tags a haplotype and is not part of any of the listed correlating clusters.

TABLE 6
Correlating Clusters for Olanzapine

| Cluster | Correlated SNPs |
|---|---|
| 01 | rs6895049; rs1047483 |
| 02 | rs1048175; rs1048166 |
| 03 | rs10511795; rs10511797 |
| 04 | rs1061015; rs1061016 |
| 05 | rs7103862; rs4505088; rs7122815; rs555867; rs10792782; |
| | rs11234192; rs1943687; rs1943691 |
| 06 | rs718805; rs10953911; rs11765060 |
| 07 | rs11737010; rs11098616 |
| 08 | rs2057899; rs12666717 |
| 09 | rs9640235; rs802022; rs10240221; rs12670106; rs1608958; |
| | rs2710157 |
| 10 | rs10503525; rs13278000 |
| 11 | rs1490407; rs1490403 |
| 12 | rs4685501; rs1554561 |
| 13 | rs4642090; rs1568982 |
| 14 | rs17622020; rs17622124 |
| 15 | rs17865434; rs17865066 |
| 16 | rs10740023; rs10821660; rs4611159; rs2061486; rs2893823 |
| 17 | rs7853207; rs3812550 |
| 18 | rs2247408; rs3819811 |
| 19 | rs3774155; rs42445 |
| 20 | rs2485528; rs4324970 |
| 21 | rs408144; rs450658; rs6711371 |
| 22 | rs688579; rs595834; rs667595 |
| 23 | rs11615961; rs708200 |
| 24 | rs2361835; rs7642607; rs7642797 |
| 25 | rs838717; rs838718 |
| 26 | rs2240717; rs917479 |
| 27 | rs9409513; rs9409514 |

TABLE 7
Correlating Clusters for Perphenazine

| Cluster | Correlated SNPs |
|---|---|
| 01 | rs4705203; rs10035432 |
| 02 | rs11750400; rs10515244 |
| 03 | rs1128687; rs1128705 |
| 04 | rs9809124; rs11705928; rs1873850 |
| 05 | rs11712897; rs11708983 |
| 06 | rs13130047; rs11731618 |
| 07 | rs17570753; rs11774231 |
| 08 | rs2058039; rs12453566 |
| 09 | rs6043211; rs6043223; rs12480304 |
| 10 | rs1253669; rs1253671 |
| 11 | rs4805590; rs12972537 |
| 12 | rs12550842; rs13256262 |
| 13 | rs1503056; rs7210687; rs1909923; rs9900811; rs203032; |
| | rs17607202; rs1503055 |
| 14 | rs1614229; rs1651285 |
| 15 | rs9852679; rs17397077 |
| 16 | rs1488519; rs17638044 |
| 17 | rs3857723; rs1859291 |
| 18 | rs9458561; rs2022996 |
| 19 | rs9671249; rs2216901 |
| 20 | rs1064833; rs2250106; rs2250338 |
| 21 | rs11772525; rs2329486 |
| 22 | rs10097662; rs2597351 |
| 23 | rs38108; rs319863 |
| 24 | rs363420; rs363343 |
| 25 | rs4849050; rs3811643 |
| 26 | rs4325897; rs4491869 |
| 27 | rs7110211; rs7944972; rs4937724 |
| 28 | rs444598; rs6762005 |
| 29 | rs6455871; rs6922278 |
| 30 | rs11002064; rs7907070 |
| 31 | rs7308849; rs7980107 |
| 32 | rs10507424; rs9530787 |
| 33 | rs17396765; rs9831415 |
| 34 | rs2247784; rs9952628 |

TABLE 8
Correlating Clusters for Quetiapine

| Cluster | Correlated SNPs |
|---|---|
| 01 | rs4711109; rs3208734; rs10447391 |
| 02 | rs6895049; rs1047483 |
| 03 | rs11188339; rs4918911; rs10882612; rs955759 |
| 04 | rs4894648; rs990634; rs10936778; rs1421422 |
| 05 | rs16915435; rs10980573 |
| 06 | rs2692677; rs1104916; rs940900; rs940898 |
| 07 | rs754710; rs11640338 |
| 08 | rs9308366; rs11647876 |
| 09 | rs404226; rs11690292 |
| 10 | rs10230286; rs11972565 |
| 11 | rs4580293; rs12607785; rs2291343 |
| 12 | rs4145506; rs12636662 |
| 13 | rs6981231; rs918; rs12675467 |
| 14 | rs11194491; rs1937972; rs12767532 |
| 15 | rs9513862; rs12874108; rs12877625 |
| 16 | rs930023; rs13278489 |
| 17 | rs4683773; rs1346134 |
| 18 | rs1836115; rs1433667 |
| 19 | rs1503056; rs9900811; rs7210687; rs1909923; rs203032; |
| | rs17607202; rs1503055 |
| 20 | rs1488519; rs17638044 |
| 21 | rs17865434; rs17865066 |
| 22 | rs10516363; rs2011495 |
| 23 | rs2073533; rs41506; rs2073532; rs41505 |
| 24 | rs10496858; rs2380943; rs4954861 |
| 25 | rs2625319; rs2585757 |
| 26 | rs2616996; rs2720851; rs2617002; rs2720811; rs2724973 |
| 27 | rs448767; rs2627222 |
| 28 | rs1878729; rs984402; rs2678780; rs3912106 |
| 29 | rs3018407; rs7110211; rs3019855; rs4937724 |
| 30 | rs4442151; rs4311658 |
| 31 | rs2485528; rs4324970 |
| 32 | rs740967; rs6466510 |
| 33 | rs3734228; rs6912580 |
| 34 | rs12960929; rs7228021 |
| 35 | rs2361835; rs7642607; rs7642797 |
| 36 | rs6957194; rs7804277 |
| 37 | rs10148780; rs8013252 |
| 38 | rs9905296; rs9912674 |
| 39 | rs998637; rs998638 |

TABLE 9
Correlating Clusters for Risperidone

| Cluster | Correlated SNPs |
|---|---|
| 01 | rs1465686; rs10069990; rs1422472 |
| 02 | rs7793570; rs6967612; rs1019000 |
| 03 | rs342368; rs1039076 |
| 04 | rs6895049; rs1047483 |
| 05 | rs4312128; rs10735885; rs10784051 |
| 06 | rs10980345; rs10816995 |
| 07 | rs4979379; rs10982201 |
| 08 | rs12619351; rs11125051 |
| 09 | rs2290492; rs11853992 |
| 10 | rs10230286; rs11972565 |
| 11 | rs4659552; rs12093821; rs7551001 |
| 12 | rs4679034; rs12492003 |
| 13 | rs11562744; rs12540990; rs4302874 |
| 14 | rs16958456; rs12600161 |
| 15 | rs3738883; rs13008804 |
| 16 | rs10508001; rs16948803 |
| 17 | rs4678793; rs2280096; rs9311104; rs2063648 |
| 18 | rs2124874; rs2168624 |
| 19 | rs4240962; rs2244496 |
| 20 | rs760761; rs2619522 |
| 21 | rs846; rs2680277 |
| 22 | rs2826668; rs2826671; rs2826672; rs2826674 |
| 23 | rs2071886; rs4149442 |
| 24 | rs12759054; rs4641353; rs6695594 |
| 25 | rs1965886; rs4775304; rs8041466 |
| 26 | rs658213; rs614728; rs583880 |
| 27 | rs1047383; rs6056229 |
| 28 | rs688579; rs667595 |
| 29 | rs6689169; rs6700378 |
| 30 | rs12960929; rs7228021 |
| 31 | rs3740511; rs7899506 |
| 32 | rs33730; rs958976 |

TABLE 10
Correlating Clusters for Ziprasidone

| Cluster | Correlated SNPs |
|---|---|
| 01 | rs992995; rs10004009 |
| 02 | rs9321977; rs10457761; rs12204734 |
| 03 | rs6895049; rs1047483 |
| 04 | rs10181727; rs1057115 |
| 05 | rs2279285; rs4757138; rs2279286; rs2279284; rs2279287; |
| | rs10766075 |
| 06 | rs4722654; rs10807838 |
| 07 | rs4977432; rs10811036 |
| 08 | rs16915435; rs10980573 |
| 09 | rs4979379; rs10982201 |
| 10 | rs11737010; rs11098616 |
| 11 | rs1377348; rs11735207; rs1453043; rs1377349 |
| 12 | rs11022220; rs12274943 |
| 13 | rs4580293; rs974080; rs12607785; rs2291343 |
| 14 | rs11721138; rs9832251; rs12635719 |
| 15 | rs281580; rs12999715 |
| 16 | rs2168123; rs1454583 |
| 17 | rs17023107; rs17023119 |
| 18 | rs934373; rs1709304 |
| 19 | rs17755910; rs17757367 |
| 20 | rs7185276; rs17768659 |
| 21 | rs11081059; rs1791398 |
| 22 | rs1386482; rs1872824 |
| 23 | rs2680832; rs1996610 |
| 24 | rs288180; rs288173 |
| 25 | rs6879227; rs2973664 |
| 26 | rs1007394; rs3118854; rs3128480 |
| 27 | rs335221; rs335244 |
| 28 | rs6773261; rs3821875 |
| 29 | rs4925199; rs4925300 |
| 30 | rs620552; rs512110 |
| 31 | rs658213; rs614728; rs9585618; rs651737; rs1452113; |
| | rs9557581; rs583880; rs7993937 |
| 32 | rs10799809; rs6698682 |
| 33 | rs921452; rs7109065 |
| 34 | rs4965881; rs7166590 |
| 35 | rs10490272; rs724605 |
| 36 | rs7395419; rs7395421 |
| 37 | rs6699880; rs7530215 |
| 38 | rs838717; rs838718 |
| 39 | rs7042481; rs9409550 |
| 40 | rs10122011; rs944478 |
| 41 | rs9578136; rs957189 |

### Example 8

### Combing the Results for Various Tagging SNPs in a Combinatorial Algorithm to Predict Response

One skilled in the art will recognize that for uncorrelated predictors (in this case alleles of various SNPs not belonging to the same correlating cluster) for a dependent variable (in this case response to antipsychotic medication, measured by change in PANSS), the influence of the predictors is independent and additive. Therefore, the genotypes for the various SNPs can be combined to develop algorithms for prediction of drug response: those in Table 2 for olanzapine, those in Table 1 for perphenazine, those in Table 3 for quetiapine, those in Table 4 for risperidone, and those Table 5 for ziprasidone.

As determined by the laws of Mendelian inheritance and confirmed by observations in the CATIE data set used for the examples presented in this specification, the inheritance of an allele at a given SNP is binary. Additionally, one skilled in the art will recognize that SNP alleles are unitary. Therefore, a given human subject will carry either 0, 1, or 2 copies of a given allele for any particular SNP listed in Tables 1-5.

Therefore, for all cases where SNPs are not members of a correlating cluster, the basic rules of multiple regression equations and straightforward mathematical principles make it true that:

If, Nᵢ=number of alleles at one SNP (SNP1) impacting response to a given drug (0, 1, 2 only); and
N₂=number of alleles of a second SNP (SNP2) impacting response to a the same drug (0, 1, 2 only); and
β₁=beta weight (slope) attributed SNP 1; and
β₂=beta weight (slope) attributed to SNP2; and
C=y intercept for PANSS-T change (by definition)=baseline change in PANSS-T for individuals carrying zero alleles of either predictor,
then the predicted response to the antipsychotic medication measured in change in PANSS-T is given by:

Expected change in PANSS-T=C+β₁N₁+β₂N₂.

Similarly, this can be generalized by the formula:

Expected change in PANSS-T=C+

where i=the number of SNPs from members of different correlating clusters selected from the one of Tables 6-10 corresponding to the particular drug.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,683,195
U.S. Pat. No. 4,683,202
U.S. Pat. No. 5,143,854
U.S. Pat. No. 5,272,071
U.S. Pat. No. 5,288,514
U.S. Pat. No. 5,288,644
U.S. Pat. No. 5,384,261
U.S. Pat. No. 5,424,186
U.S. Pat. No. 5,451,683
U.S. Pat. No. 5,491,224
U.S. Pat. No. 5,510,270
U.S. Pat. No. 5,527,681
U.S. Pat. No. 5,776,688
U.S. Pat. No. 5,800,998
U.S. Pat. No. 5,837,832
U.S. Pat. No. 5,858,659
U.S. Patent Publication No. 2003/0108938
U.S. Patent Publication No. 2004/0014095
U.S. Patent Publication No. 2006/0177851
U.S. Patent Publication No. 2009/0012371
PCT US/93/04145
WO 91/06667
WO 92/10092
WO 95/11995
WO 98/20019
WO 99/57318
WO 2009/092032
WO 2009/089120
Albertson et al., Breast Cancer Research and Treatment 78:289-298 (2003).
Alderborn et al., Genome Research 10(8):1249-1258 (2000).
Ausubel et al., Current Protocols in Molecular Biology, John Wiley & Sons, N.Y. (2003).
Badner et al., Mol. Psychiatry. 7:405-411 (2002).
Bao et al., PolymiRTS Database: linking polymorphisms in microRNA target sites with complex traits. Nucleic Acids Res. 35(Database issue), D51-D54 (2007).
Barrett et al., Haploview: analysis and visualization of LD and haplotype maps. Bioinformatics. 21(2), 263-265 (2005).
Barrett, Haploview: Visualization and analysis of SNP genotype data. Cold Spring Harb.Protoc. 2009(10), db-(2009).
Chen et al., Genome Research 9(5):492-498 (1999).
Church and Gilbert, Proc. Natl. Acad. Sci. USA 81:1991-1995 (1988).
Cooper-Casey et al., Mol. Psychiatry. 10:651-656 (2005).
Cotton et al., Proc. Natl. Acad. Sci. USA 85:4397-4401 (1985).
Devlin et al., Mol. Psychiatry. 7:689-694 (2002).
Eckert et al., PCR Methods and Applications 1:17 (1991).
Eichelbaum et al., Clin. Exp. Pharmacol. Physiol. 23:983-985 (1996).
Fallin et al., Am. J. Hum. Genet. 77:918-936 (2005).
Fallin et al., Am. J. Hum. Genet. 73:601-611 (2003).
Flavell et al., Cell 15:25 (1978).
Geever et al., Proc. Natl. Acad. Sci. USA 78:5081 (1981).
Guatelli et al., Proc. Nat. Acad. Sci. USA 87:1874 (1990).
Guttmacher and Collins JAMA 294:1399-402 (2005).
Hamer and Simpson, Last observation carried forward versus mixed models in the analysis of psychiatric clinical trials. Am. J. Psychiatry 166(6), 639-641 (2009).
Jablensky, Mol. Psychiatry. 11: 815-836 (2006).
Kane, Pharmacologic treatment of schizophrenia. Biol. Psychiatry 46(10), 1396-1408 (1999).
Kay et al., Schizophr. Bull. 13:261-276 (1987).
Kay et al., Br. J. Psychiatry Suppl: 59-67 (1989).
Kirov et al., J. Clin. Invest. 115:1440-1448 (2005).
Klein et al., Proc. Natl. Acad. Sci. USA 96:4494-4499 (1999).
Kwoh et al., Proc. Natl. Acad. Sci. USA 86:1173 (1989).
Landegren et al., Science 241:1077 (1988).
Leucht et al., Schizophr. Res. 79:231-238 (2005).
Lieberman et al., Effectiveness of antipsychotic drugs in patients with chronic schizophrenia. N.Engl.J.Med. 353(12), 1209-1223 (2005).
Linder et al., Clin. Chem. 43:254-266 (1997).
Liu et al., Pharmacogenomics. 13:1227-1237 (2012).
Maniatis et al., Proc. Natl. Acad. Sci. USA 99:2228-2233 (2002).
Manolio et al., New models of collaboration in genome-wide association studies: the Genetic Association Information Network. Nat.Genet. 39(9), 1045-1051 (2007).
Mattila et al., Nucleic Acids Res. 19:4967 (1991).
McClay et al., Genome-wide pharmacogenomic analysis of response to treatment with antipsychotics. Mol.Psychiatry 16(1), 76-85 (2011).
McPherson et al., PCR Basics: From Background to Bench, Springer-Verlag, (2000).
Meltzer and Huang, In vivo actions of atypical antipsychotic drug on serotonergic and dopaminergic systems. Prog.Brain Res. 172, 177-197 (2008).
Morton et al., Proc. Natl. Acad. Sci. USA 98(9):5217-21 (2001).
Myers et al., Science 230:1242 (1985).
Nath and Johnson, Biotechnic. Histochem. 73(1):6-22 (1998).
Need et al., Pharmacogenetics of antipsychotic response in the CATIE trial: a candidate gene analysis. Eur.J.Hum.Genet. 17(7), 946-957 (2009a).
Need et al., A genome-wide investigation of SNPs and CNVs in schizophrenia. PLoS.Genet. 5(2), e1000373-(2009b).
Nielsen et al., Bioconjugate Chemistry, The American Chemical Society, 5:1 (1994).
Norton et al., Curr. Opin. Psychiatry. 19:158-164 (2006).
Orita et al., Proc. Natl. Acad. Sci. USA 86:2766-2770 (1989).
Owen et al., Mol. Psychiatry. 9:14-27 (2004).
PCR (eds. McPherson et al., IRL Press, Oxford).
PCR Primer: A Laboratory Manual, Dieffenbach and Dveksler, (Eds.).
Practice Guideline for the Treatment of Patients With Schizophrenia American Psychiatric
Association, Second Edition, American Psychiatric Association (2004).
Prince et al., Genome Res. 11:152-162 (2001).
Purcell et al., PLINK: a tool set for whole-genome association and population-based linkage analyses. Am.J.Hum.Genet. 81(3), 559-575 (2007).
Raca et al., Genet Test 8(4):387-94 (2004).
Redon et al., Nature 444(7118):444-54 (2006).
Saiki et al., Nature 324:163-166 (1986).
Sanger et al., Proc. Natl. Acad. Sci. USA 74:5463-5467 (1977).
Schafer et al., Nat. Biotechnol. 15:33-39 (1995).
Sheffield et al., Proc. Natl. Acad. Sci. USA 86:232-236 (1989).
Snijders et al., Nat. Genetics 29:263-264 (2001).
Snijders et al., “BAC microarray based comparative genomic hybridization,” in: Zhao et al. (eds), Bacterial Artificial Chromosomes: Methods and Protocols, Methods in Molecular Biology, Humana Press (2002).
Stone et al., Nature 455(7210):237-41 (2008).
Stoneking et al., Am. J. Hum. Genet. 48:370-382 (1991).
Stroup et al., The National Institute of Mental Health Clinical Antipsychotic Trials of Intervention Effectiveness (CATIE) project: schizophrenia trial design and protocol development. Schizophr.Bull. 29(1), 15-31 (2003).
Sullivan et al., Genomewide association for schizophrenia in the CATIE study: results of stage 1. Mol.Psychiatry 13(6), 570-584 (2008).
Tapper et al., Proc. Natl. Acad. Sci. USA 102(33):11835-11839 (2005).
The International HapMap Consortium, Nature 426:789-796 (2003).
The International HapMap Consortium, Nature 437:1299-1320 (2005).
Underhill et al., Genome Res. 7:996-1005 (1997).
van den Oord et al., A systematic method for estimating individual responses to treatment with antipsychotics in CATIE. Schizophr.Res. 107(1), 13-21 (2009).
Walsh et al., Science 320(5875):539-43 (2008).
Wang et al., Pharmacogenetics 13:555-64 (2003).
Weinshilboum and Wang, Nature Rev. Drug Discovery 3:739-748 (2004).
Weinshilboum and Wang, Annu. Rev. Genomics Hum. Genet. 7:223-45 (2006).
Wheeless et al., Cytometry 17:319-326 (1994).
Williams et al., Hum. Mol. Genet. 8:1729-1739 (1999).
Wu and Wallace, Genomics 4:560 (1989).
Ziebarth et al., PolymiRTS Database 2.0: linking polymorphisms in microRNA target sites with human diseases and complex traits. Nucleic Acids Res. 40(Database issue), D216-D221 (2012).

What is claimed is:

1. A method of detecting the presence of a polymorphism in the PSMD14, LRP1B, or TMEFF2 gene and administering a treatment to a human subject, the method comprising:
(a) obtaining a genomic sample from a human subject having or at risk of developing SZ;
(b) detecting the haplotype tagged by the “A” allele of rs9713, the haplotype tagged by the “C” allele of rs874295, or the haplotype tagged by the “C” allele of rs3738883 in the genomic sample;
(c) identifying the subject having the haplotype tagged by the “A” allele of rs9713, the haplotype tagged by the “C” allele of rs874295, or the haplotype tagged by the “C” allele of rs3738883 in the genomic sample as likely to have an improved response to risperidone as compared to control subject; and
(d) administering a treatment comprising risperidone to the subject with the haplotype tagged by the “A” allele of rs9713, the haplotype tagged by the “C” allele of rs874295, or the haplotype tagged by the “C” allele of rs3738883.

2. A method of treating a human subject having or at risk of developing SZ comprising administering an effective amount of risperidone to a subject determined to have a haplotype tagged by the “A” allele of rs9713, the haplotype tagged by the “C” allele of rs874295, or the haplotype tagged by the “C” allele of rs3738883.

3. A method of detecting the presence of a polymorphism in and administering a treatment to a human subject, the method comprising:
(a) obtaining a genomic sample from a human subject having or at risk of developing SZ;
(b) detecting the haplotype tagged by an allele selected from those provided in Table 1A in the genomic sample;
(c) identifying the subject having the haplotype tagged by the allele provided in Table 1A in the genomic sample as likely to have an improved response to olanzapine as compared to control subject; and
(d) administering a treatment comprising olanzapine to the subject with the haplotype tagged by the allele provided in Table 1A.

4. A method of treating a human subject having or at risk of developing SZ comprising administering an effective amount of olanzapine to a subject determined to have a haplotype tagged by an allele selected from those provided in Table 1A.

5. The method of claim 3, further comprising detecting the haplotype tagged by two or more alleles selected from those provided in Table 1A.

6. The method of claim 5, wherein said two or more alleles are alleles from two or more different correlated clusters selected from those provided in Table 6.

7. A method of detecting the presence of a polymorphism in and administering a treatment to a human subject, the method comprising:
(a) obtaining a genomic sample from a human subject having or at risk of developing SZ;
(b) detecting the haplotype tagged by an allele selected from those provided in Table 1B in the genomic sample;
(c) identifying the subject having the haplotype tagged by the allele provided in Table 1B in the genomic sample as likely to have a poor response to olanzapine as compared to control subject; and
(d) administering an antipsychotic treatment other than olanzapine to the subject with the haplotype tagged by the allele provided in Table 1B.

8. A method of treating a human subject having or at risk of developing SZ comprising administering an effective amount of an antipsychotic agent that is not olanzapine to a subject determined to have a haplotype tagged by an allele selected from those provided in Table 1B.

9. The method of claim 7, further comprising detecting the haplotype tagged by two or more alleles selected from those provided in Table 1B.

10. The method of claim 9, wherein said two or more alleles are alleles from two or more different correlated clusters selected from those provided in Table 6.

11. The method of claim 7, comprising administering perphenazine, quetiapine, risperidone or ziprasidone to the subject.

12. A method of detecting the presence of a polymorphism in the CSMD1 or PTPRN2 gene and administering a treatment to a human subject, the method comprising:
(a) obtaining a genomic sample from a human subject having or at risk of developing SZ;
(b) detecting the haplotype tagged by the “A” allele of rs17070785 or the haplotype tagged by the “C” allele of rs221253 in the genomic sample;
(c) identifying the subject having the haplotype tagged by the “A” allele of rs17070785 or the haplotype tagged by the “C” allele of rs221253 in the genomic sample as likely to have an improved response to olanzapine as compared to control subject; and
(d) administering a treatment comprising olanzapine to the subject with the haplotype tagged by the “A” allele of rs17070785 or the haplotype tagged by the “C” allele of rs221253.

13. A method of treating a human subject having or at risk of developing SZ comprising administering an effective amount of olanzapine to a subject determined to have a the haplotype tagged by the “A” allele of rs17070785 or the haplotype tagged by the “C” allele of rs221253.

14. A method of detecting the presence of a polymorphism in the PLAGL1 gene and administering an antipsychotic treatment to a human subject, the method comprising:
(a) obtaining a genomic sample from a human subject having or at risk of developing SZ;
(b) detecting the haplotype tagged by the “C” allele of rs2247408 or the haplotype tagged by the “A” allele of rs3819811 in the genomic sample;
(c) identifying the subject having the haplotype tagged by the “C” allele of rs2247408 or the haplotype tagged by the “A” allele of rs3819811 in the genomic sample as likely to have a poor response to olanzapine as compared to control subject; and
(d) administering an antipsychotic treatment other than olanzapine to the subject with the haplotype tagged by the “C” allele of rs2247408 or the haplotype tagged by the “A” allele of rs3819811.

15. The method of claim 14, comprising administering perphenazine, quetiapine, risperidone or ziprasidone to the subject.

16. A method of treating a human subject having or at risk of developing SZ comprising administering an effective amount of an antipsychotic agent that is not olanzapine to a subject determined to have a haplotype tagged by the “C” allele of rs2247408 or the haplotype tagged by the “A” allele of rs3819811.

17. A method of detecting the presence of a polymorphism in and administering a treatment to a human subject, the method comprising:
(a) obtaining a genomic sample from a human subject having or at risk of developing SZ;
(b) detecting the haplotype tagged by an allele selected from those provided in Table 2A in the genomic sample;
(c) identifying the subject having the haplotype tagged by the allele provided in Table 2A in the genomic sample as likely to have an improved response to perphenazine as compared to control subject; and
(d) administering a treatment comprising perphenazine to the subject with the haplotype tagged by the allele provided in Table 2A.

18. A method of treating a human subject having or at risk of developing SZ comprising administering an effective amount of perphenazine to a subject determined to have a haplotype tagged by an allele selected from those provided in Table 2A.

19. The method of claim 17, further comprising detecting the haplotype tagged by two or more alleles selected from those provided in Table 2A.

20. The method of claim 19, wherein said two or more alleles are alleles from two or more different correlated clusters selected from those provided in Table 7.

21. A method of detecting the presence of a polymorphism in and administering a treatment to a human subject, the method comprising:
(a) obtaining a genomic sample from a human subject having or at risk of developing SZ;
(b) detecting the haplotype tagged by an allele selected from those provided in Table 2B in the genomic sample;
(c) identifying the subject having the haplotype tagged by the allele provided in Table 2B in the genomic sample as likely to have a poor response to perphenazine as compared to control subject; and
(d) administering an antipsychotic treatment other than perphenazine to the subject with the haplotype tagged by the allele provided in Table 2B.

22. A method of treating a human subject having or at risk of developing SZ comprising administering an effective amount of an antipsychotic agent that is not perphenazine to a subject determined to have a haplotype tagged by an allele selected from those provided in Table 2B.

23. The method of claim 21, further comprising detecting the haplotype tagged by two or more alleles selected from those provided in Table 2B.

24. The method of claim 23, wherein said two or more alleles are alleles from two or more different correlated clusters selected from those provided in Table 7.

25. The method of claim 21, comprising administering olanzapine, quetiapine, risperidone or ziprasidone to the subject.

26. A method of detecting the presence of a polymorphism in the MCPH1, PRKCE, CDH13, or SKOR2 gene and administering a treatment to a human subject, the method comprising:
(a) obtaining a genomic sample from a human subject having or at risk of developing SZ;
(b) detecting the haplotype tagged by the “C” allele of rs11774231, the haplotype tagged by the “C” allele of rs2278773, the haplotype tagged by the “A” allele of rs17570753, the haplotype tagged by the “C” allele of rs2116971, or the haplotype tagged by the “G” allele of rs9952628 in the genomic sample;
(c) identifying the subject having the haplotype tagged by the “C” allele of rs11774231, the haplotype tagged by the “C” allele of rs2278773, the haplotype tagged by the “A” allele of rs17570753, the haplotype tagged by the “C” allele of rs2116971, or the haplotype tagged by the “G” allele of rs9952628 in the genomic sample as likely to have an improved response to perphenazine as compared to control subject; and
(d) administering a treatment comprising perphenazine to the subject with the haplotype tagged by the “C” allele of rs11774231, the haplotype tagged by the “C” allele of rs2278773, the haplotype tagged by the “A” allele of rs17570753, the haplotype tagged by the “C” allele of rs2116971, or the haplotype tagged by the “G” allele of rs9952628.

27. A method of treating a human subject having or at risk of developing SZ comprising administering an effective amount of perphenazine to a subject determined to have a haplotype tagged by the “C” allele of rs11774231, the haplotype tagged by the “C” allele of rs2278773, the haplotype tagged by the “A” allele of rs17570753, the haplotype tagged by the “C” allele of rs2116971, or the haplotype tagged by the “G” allele of rs9952628.

28. A method of detecting the presence of a polymorphism in the MAML3 gene and administering a treatment to a human subject, the method comprising:
(a) obtaining a genomic sample from a human subject having or at risk of developing SZ;
(b) detecting the haplotype tagged by the “A” allele of rs 11100483 in the genomic sample;
(c) identifying the subject having the haplotype tagged by the “A” allele of rs11100483 in the genomic sample as likely to have a poor response to perphenazine as compared to control subject; and
(d) administering an antipsychotic treatment other than perphenazine to the subject with the haplotype tagged by the “A” allele of rs11100483.

29. A method of treating a human subject having or at risk of developing SZ comprising administering an effective amount of an antipsychotic agent that is not perphenazine to a subject determined to have a haplotype tagged by the “A” allele of rs11100483.

30. The method of claim 28, comprising administering olanzapine, quetiapine, risperidone or ziprasidone to the subject.

31. A method of detecting the presence of a polymorphism in and administering a treatment to a human subject, the method comprising:
(a) obtaining a genomic sample from a human subject having or at risk of developing SZ;
(b) detecting the haplotype tagged by an allele selected from those provided in Table 3A in the genomic sample;
(c) identifying the subject having the haplotype tagged by the allele provided in Table 3A in the genomic sample as likely to have an improved response to quetiapine as compared to control subject; and
(d) administering a treatment comprising quetiapine to the subject with the haplotype tagged by the allele provided in Table 3A.

32. A method of treating a human subject having or at risk of developing SZ comprising administering an effective amount of quetiapine to a subject determined to have a haplotype tagged by an allele selected from those provided in Table 3A.

33. The method of claim 31, further comprising detecting the haplotype tagged by two or more alleles selected from those provided in Table 3A.

34. The method of claim 33, wherein said two or more alleles are alleles from two or more different correlated clusters selected from those provided in Table 8.

35. A method of detecting the presence of a polymorphism in and administering a treatment to a human subject, the method comprising:
(a) obtaining a genomic sample from a human subject having or at risk of developing SZ;
(b) detecting the haplotype tagged by an allele selected from those provided in Table 3B in the genomic sample;
(c) identifying the subject having the haplotype tagged by the allele provided in Table 3B in the genomic sample as likely to have a poor response to quetiapine as compared to control subject; and
(d) administering an antipsychotic treatment other than quetiapine to the subject with the haplotype tagged by the allele provided in Table 3B.

36. A method of treating a human subject having or at risk of developing SZ comprising administering an effective amount of an antipsychotic agent that is not quetiapine to a subject determined to have a haplotype tagged by an allele selected from those provided in Table 3B.

37. The method of claim 35, further comprising detecting the haplotype tagged by two or more alleles selected from those provided in Table 3B.

38. The method of claim 37, wherein said two or more alleles are alleles from two or more different correlated clusters selected from those provided in Table 8.

39. The method of claim 35, comprising administering olanzapine, perphenazine, risperidone or ziprasidone to the subject.

40. A method of detecting the presence of a polymorphism in the KCNMA1 gene and administering a treatment to a human subject, the method comprising:
(a) obtaining a genomic sample from a human subject having or at risk of developing SZ;
(b) detecting the haplotype tagged by the “C” allele of rs35793;
(c) identifying the subject having the haplotype tagged by the “C” allele of rs35793 in the genomic sample as likely to have a poor response to quetiapine as compared to control subject; and
(d) administering an antipsychotic treatment other than quetiapine to the subject with the haplotype tagged by the “C” allele of rs35793.

41. A method of treating a human subject having or at risk of developing SZ comprising administering an effective amount of an antipsychotic agent that is not quetiapine to a subject determined to have a haplotype tagged by the “C” allele of rs35793.

42. The method of claim 40, comprising administering olanzapine, perphenazine, risperidone or ziprasidone to the subject.

43. A method of detecting the presence of a polymorphism in and administering a treatment to a human subject, the method comprising:
(a) obtaining a genomic sample from a human subject having or at risk of developing SZ;
(b) detecting the haplotype tagged by an allele selected from those provided in Table 4A in the genomic sample;
(c) identifying the subject having the haplotype tagged by the allele provided in Table 4A in the genomic sample as likely to have an improved response to risperidone as compared to control subject; and
(d) administering a treatment comprising risperidone to the subject with the haplotype tagged by the allele provided in Table 4A.

44. A method of treating a human subject having or at risk of developing SZ comprising administering an effective amount of risperidone to a subject determined to have a haplotype tagged by an allele selected from those provided in Table 4A.

45. The method of claim 43, further comprising detecting the haplotype tagged by two or more alleles selected from those provided in Table 4A.

46. The method of claim 45, wherein said two or more alleles are alleles from two or more different correlated clusters selected from those provided in Table 9.

47. A method of detecting the presence of a polymorphism in and administering a treatment to a human subject, the method comprising:
(a) obtaining a genomic sample from a human subject having or at risk of developing SZ;
(b) detecting the haplotype tagged by an allele selected from those provided in Table 4B in the genomic sample;
(c) identifying the subject having the haplotype tagged by the allele provided in Table 4B in the genomic sample as likely to have a poor response to risperidone as compared to control subject; and
(d) administering an antipsychotic treatment other than risperidone to the subject with the haplotype tagged by the allele provided in Table 4B.

48. A method of treating a human subject having or at risk of developing SZ comprising administering an effective amount of an antipsychotic agent that is not risperidone to a subject determined to have a haplotype tagged by an allele selected from those provided in Table 4B.

49. The method of claim 47, further comprising detecting the haplotype tagged by two or more alleles selected from those provided in Table 4B.

50. The method of claim 49, wherein said two or more alleles are alleles from two or more different correlated clusters selected from those provided in Table 9.

51. The method of claim 47, comprising administering olanzapine, perphenazine, quetiapine or ziprasidone to the subject.

52. A method of detecting the presence of a polymorphism in the AGAP 1 or NPAS3 gene and administering a treatment to a human subject, the method comprising:
(a) obtaining a genomic sample from a human subject having or at risk of developing SZ;
(b) detecting the haplotype tagged by the “C” allele of rs1869295 or the haplotype tagged by the “C” allele of rs 1315115 in the genomic sample;
(c) identifying the subject having the haplotype tagged by the “C” allele of rs1869295 or the haplotype tagged by the “C” allele of rs1315115 in the genomic sample as likely to have a poor response to risperidone as compared to control subject; and
(d) administering an antipsychotic treatment other than risperidone to the subject with the haplotype tagged by the “C” allele of rs 1869295 or the haplotype tagged by the “C” allele of rs1315115.

53. A method of treating a human subject having or at risk of developing SZ comprising administering an effective amount of an antipsychotic agent that is not risperidone to a subject determined to have a haplotype tagged by the “C” allele of rs1869295 or the haplotype tagged by the “C” allele of rs1315115.

54. The method of claim 52, comprising administering olanzapine, perphenazine, quetiapine or ziprasidone to the subject.

55. A method of detecting the presence of a polymorphism in and administering a treatment to a human subject, the method comprising:
(a) obtaining a genomic sample from a human subject having or at risk of developing SZ;
(b) detecting the haplotype tagged by an allele selected from those provided in Table 5A in the genomic sample;
(c) identifying the subject having the haplotype tagged by the allele provided in Table 5A in the genomic sample as likely to have an improved response to ziprasidone as compared to control subject; and
(d) administering a treatment comprising ziprasidone to the subject with the haplotype tagged by the allele provided in Table 5A.

56. A method of treating a human subject having or at risk of developing SZ comprising administering an effective amount of ziprasidone to a subject determined to have a haplotype tagged by an allele selected from those provided in Table 5A.

57. The method of claim 55, further comprising detecting the haplotype tagged by two or more alleles selected from those provided in Table 5A.

58. The method of claim 57, wherein said two or more alleles are alleles from two or more different correlated clusters selected from those provided in Table 10.

59. A method of detecting the presence of a polymorphism in and administering a treatment to a human subject, the method comprising:
(a) obtaining a genomic sample from a human subject having or at risk of developing SZ;
(b) detecting the haplotype tagged by an allele selected from those provided in Table 5B in the genomic sample;
(c) identifying the subject having the haplotype tagged by the allele provided in Table 5B in the genomic sample as likely to have a poor response to ziprasidone as compared to control subject; and
(d) administering an antipsychotic treatment other than ziprasidone to the subject with the haplotype tagged by the allele provided in Table 5B.

60. A method of treating a human subject having or at risk of developing SZ comprising administering an effective amount of an antipsychotic agent that is not ziprasidone to a subject determined to have a haplotype tagged by an allele selected from those provided in Table 5B.

61. The method of claim 59, further comprising detecting the haplotype tagged by two or more alleles selected from those provided in Table 5B.

62. The method of claim 61, wherein said two or more alleles are alleles from two or more different correlated clusters selected from those provided in Table 10.

63. The method of claim 59, comprising administering olanzapine, perphenazine, quetiapine or risperidone to the subject.

64. A method of detecting the presence of a polymorphism in the CDH4, LYN, or CNTN4 gene and administering a treatment to a human subject, the method comprising:
(a) obtaining a genomic sample from a human subject having or at risk of developing SZ;
(b) detecting the haplotype tagged by the “A” allele of rs4925300, the haplotype tagged by the “C” allele of rs 1546519, or the haplotype tagged by the “A” allele of rs17194378 in the genomic sample;
(c) identifying the subject having the haplotype tagged by the “A” allele of rs4925300, the haplotype tagged by the “C” allele of rs1546519, or the haplotype tagged by the “A” allele of rs17194378 in the genomic sample as likely to have an improved response to ziprasidone as compared to control subject; and
(d) administering a treatment comprising ziprasidone to the subject with the haplotype tagged by the “A” allele of rs4925300, the haplotype tagged by the “C” allele of rs1546519, or the haplotype tagged by the “A” allele of rs17194378.

65. A method of treating a human subject having or at risk of developing SZ comprising administering an effective amount of ziprasidone to a subject determined to have a haplotype tagged by the “A” allele of rs4925300, the haplotype tagged by the “C” allele of rs1546519, or the haplotype tagged by the “A” allele of rs17194378.

66. A method of detecting the presence of a polymorphism in the NALCN gene and administering a treatment to a human subject, the method comprising:
(a) obtaining a genomic sample from a human subject having or at risk of developing SZ;
(b) detecting the haplotype tagged by the “C” allele of rs9585618 in the genomic sample;
(c) identifying the subject having the haplotype tagged by the “C” allele of rs9585618 in the genomic sample as likely to have a poor response to ziprasidone as compared to control subject; and
(d) administering an antipsychotic treatment other than ziprasidone to the subject with the haplotype tagged by the “C” allele of rs9585618.

67. A method of treating a human subject having or at risk of developing SZ comprising administering an effective amount of an antipsychotic agent that is not ziprasidone to a subject determined to have a haplotype tagged by the “C” allele of rs9585618.

68. The method of claim 66, comprising administering olanzapine, perphenazine, quetiapine or risperidone to the subject.

69. The method of any one of claims 3-68, wherein the subject has early, intermediate, or aggressive SZ.

70. The method of any one of claims 3-68, wherein the subject has one or more risk factors associated with SZ.

71. The method of any one of claims 3-68, wherein the haplotype tagged by an allele comprises determining the number of alleles tagging the haplotype in the subject.

72. The method of any one of claims 3-68, wherein the subject has a relative afflicted with SZ or a genetically-based phenotypic trait associated with risk for SZ.

73. The method of any one of claims 3-68, wherein the subject is Caucasian or comprises European ancestry.

74. A method of identifying and administering a treatment to a human subject, the method comprising:
(a) obtaining a genomic sample from a human subject having or at risk of developing SZ;
(b) detecting two or more haplotypes tagged by alleles selected from those provided in Tables 1-5 in the genomic sample;
(c) calculating a predicted treatment efficacy for at least two drugs selected from the group consisting of olanzapine, perphenazine, quetiapine, risperidone, and ziprasidone;
(d) ranking the predicted efficacy of the at least two drugs; and
(e) administering a treatment comprising the drug with the highest predicted efficiency to the subject based on said ranking.

75. The method of claim 74, wherein detecting two or more haplotypes tagged by an allele comprises determining the number of alleles tagging the two or more haplotypes in the subject.

76. The method of claim 75, wherein calculating a predicted treatment efficacy for a given drug comprises assigning a weighted value to each haplotype influencing response to that drug and multiplying the weighted value by the number of alleles tagging the haplotype in the subject.

77. The method of claim 75, wherein calculating a predicted treatment efficacy for a given drug comprises using the equation:
P=C+ΣᵢβᵢNᵢ
wherein P is the predicted treatment efficacy measured in change in PANSS-T; C is the change in PANSS-T for individuals carrying zero alleles of any response-predicting haplotype for the drug, β is the weighted value for at least a first haplotype measured in PANSS-T; N is the number of alleles tagging at least the first haplotype; and i is the number of haplotypes detected.

78. The method of claim 74, comprising a predicted treatment efficacy for three, four or five drugs selected from the group consisting of olanzapine, perphenazine, quetiapine, risperidone, and ziprasidone.

79. The method of any one of claims 74-78, wherein the subject has early, intermediate, or aggressive SZ.

80. The method of any one of claims 74-78, wherein the subject has one or more risk factors associated with SZ.

81. The method of any one of claims 74-78, wherein the subject has a relative afflicted with SZ or a genetically-based phenotypic trait associated with risk for SZ.

82. The method of any one of claims 74-78, wherein the subject is Caucasian or comprises European ancestry.

83. An in vitro method of detecting the presence of a polymorphism in a human subject, the method comprising:
(a) detecting the haplotype tagged by an allele selected from those provided in Table 1A in a genomic sample from a human subject having or at risk of developing SZ; and
(b) identifying the subject having the haplotype tagged by the allele provided in Table 1A in the genomic sample as likely to have an improved response to olanzapine as compared to a control subject.

84. The method of claim 83, further comprising detecting the haplotype tagged by two or more alleles selected from those provided in Table 1A.

85. The method of claim 84, wherein said two or more alleles are alleles from two or more different correlated clusters selected from those provided in Table 6.

86. A composition comprising olanzapine for use in treating a human subject having or at risk of developing SZ, the human subject having a haplotype tagged by an allele provided in Table 1A.

87. The composition of claim 86, wherein the human subject has a haplotype tagged by two or more alleles selected from those provided in Table 1A.

88. The composition of claim 87, wherein said two or more alleles are alleles from two or more different correlated clusters selected from those provided in Table 6.

89. An in vitro method of detecting the presence of a polymorphism in a human subject, the method comprising:
(a) detecting the haplotype tagged by an allele selected from those provided in Table 1B in a genomic sample from a human subject having or at risk of developing SZ; and
(b) identifying the subject having the haplotype tagged by the allele provided in Table 1B in the genomic sample as likely to have a poor response to olanzapine as compared to a control subject.

90. The method of claim 89 further comprising detecting the haplotype tagged by two or more alleles selected from those provided in Table 1B.

91. The method of claim 90, wherein said two or more alleles are alleles from two or more different correlated clusters selected from those provided in Table 6.

92. A composition comprising an antipsychotic agent that is not olanzapine for use in treating a human subject having or at risk of developing SZ, the human subject having a haplotype tagged by an allele provided in Table 1B.

93. The composition of claim 92, comprising perphenazine, quetiapine, risperidone or ziprasidone.

94. The composition of claim 92, wherein the human subject has a haplotype tagged by two or more alleles selected from those provided in Table 1B.

95. The composition of claim 94, wherein said two or more alleles are alleles from two or more different correlated clusters selected from those provided in Table 6.

96. An in vitro method of detecting the presence of a polymorphism in a human subject, the method comprising:
(a) detecting a haplotype tagged by the “A” allele of rs17070785 or a haplotype tagged by the “C” allele of rs221253 in a genomic sample from a human subject having or at risk of developing SZ; and
(b) identifying the subject having the haplotype tagged by the “A” allele of rs17070785 or the haplotype tagged by the “C” allele of rs221253 in the genomic sample as likely to have an improved response to olanzapine as compared to a control subject.

97. A composition comprising olanzapine for use in treating a human subject having or at risk of developing SZ, the human subject having a haplotype tagged by the “A” allele of rs17070785 or a haplotype tagged by the “C” allele of rs221253.

98. An in vitro method of detecting the presence of a polymorphism in a human subject, the method comprising:
(a) detecting a haplotype tagged by the “C” allele of rs2247408 or the haplotype tagged by the “A” allele of rs3819811 in a genomic sample from a human subject having or at risk of developing SZ; and
(b) identifying the subject having the haplotype tagged by the “C” allele of rs2247408 or the haplotype tagged by the “A” allele of rs3819811 in the genomic sample as likely to have poor response to olanzapine as compared to a control subject.

99. A composition comprising an antipsychotic agent that is not olanzapine for use in treating a human subject having or at risk of developing SZ, the human subject having a haplotype tagged by the “C” allele of rs2247408 or the haplotype tagged by the “A” allele of rs3819811.

100. The composition of claim 99, comprising perphenazine, quetiapine, risperidone or ziprasidone.

101. An in vitro method of detecting the presence of a polymorphism in a human subject, the method comprising:
(a) detecting the haplotype tagged by an allele selected from those provided in Table 2A in a genomic sample from a human subject having or at risk of developing SZ; and
(b) identifying the subject having the haplotype tagged by the allele provided in Table 2A in the genomic sample as likely to have an improved response to perphenazine as compared to a control subject.

102. The method of claim 101, further comprising detecting the haplotype tagged by two or more alleles selected from those provided in Table 2A.

103. The method of claim 102, wherein said two or more alleles are alleles from two or more different correlated clusters selected from those provided in Table 7.

104. A composition comprising perphenazine for use in treating a human subject having or at risk of developing SZ, the human subject having a haplotype tagged by an allele provided in Table 2A.

105. The composition of claim 104, wherein the human subject has a haplotype tagged by two or more alleles selected from those provided in Table 2A.

106. The composition of claim 105, wherein said two or more alleles are alleles from two or more different correlated clusters selected from those provided in Table 7.

107. An in vitro method of detecting the presence of a polymorphism in a human subject, the method comprising:
(a) detecting the haplotype tagged by an allele selected from those provided in Table 2B in a genomic sample from a human subject having or at risk of developing SZ; and
(b) identifying the subject having the haplotype tagged by the allele provided in Table 2B in the genomic sample as likely to have a poor response to perphenazine as compared to a control subject.

108. The method of claim 107 further comprising detecting the haplotype tagged by two or more alleles selected from those provided in Table 2B.

109. The method of claim 108, wherein said two or more alleles are alleles from two or more different correlated clusters selected from those provided in Table 7.

110. A composition comprising an antipsychotic agent that is not perphenazine for use in treating a human subject having or at risk of developing SZ, the human subject having a haplotype tagged by an allele provided in Table 2B.

111. The composition of claim 110, comprising olanzapine, quetiapine, risperidone or ziprasidone.

112. The composition of claim 110, wherein the human subject has a haplotype tagged by two or more alleles selected from those provided in Table 2B.

113. The composition of claim 112, wherein said two or more alleles are alleles from two or more different correlated clusters selected from those provided in Table 7.

114. An in vitro method of detecting the presence of a polymorphism in a human subject, the method comprising:
(a) detecting a haplotype tagged by the “C” allele of rs11774231, the haplotype tagged by the “C” allele of rs2278773, the haplotype tagged by the “A” allele of rs17570753, the haplotype tagged by the “C” allele of rs2116971, or the haplotype tagged by the “G” allele of rs9952628 in a genomic sample from a human subject having or at risk of developing SZ; and
(b) identifying the subject having the haplotype tagged by the “C” allele of rs11774231, the haplotype tagged by the “C” allele of rs2278773, the haplotype tagged by the “A” allele of rs17570753, the haplotype tagged by the “C” allele of rs2116971, or the haplotype tagged by the “G” allele of rs9952628 in the genomic sample as likely to have an improved response to perphenazine as compared to a control subject.

115. A composition comprising perphenazine for use in treating a human subject having or at risk of developing SZ, the human subject having a haplotype tagged by the “C” allele of rs11774231, the haplotype tagged by the “C” allele of rs2278773, the haplotype tagged by the “A” allele of rs17570753, the haplotype tagged by the “C” allele of rs2116971, or the haplotype tagged by the “G” allele of rs9952628.

116. An in vitro method of detecting the presence of a polymorphism in a human subject, the method comprising:
(a) detecting a haplotype tagged by the “A” allele of rs11100483 in a genomic sample from a human subject having or at risk of developing SZ; and
(b) identifying the subject having the haplotype tagged by the “A” allele of rs11100483 in the genomic sample as likely to have poor response to perphenazine as compared to a control subject.

117. A composition comprising an antipsychotic agent that is not perphenazine for use in treating a human subject having or at risk of developing SZ, the human subject having a haplotype tagged by the “A” allele of rs11100483.

118. The composition of claim 117, comprising olanzapine, quetiapine, risperidone or ziprasidone.

119. An in vitro method of detecting the presence of a polymorphism in a human subject, the method comprising:
(a) detecting the haplotype tagged by an allele selected from those provided in Table 3A in a genomic sample from a human subject having or at risk of developing SZ; and
(b) identifying the subject having the haplotype tagged by the allele provided in Table 3A in the genomic sample as likely to have an improved response to quetiapine as compared to a control subject.

120. The method of claim 119, further comprising detecting the haplotype tagged by two or more alleles selected from those provided in Table 3A.

121. The method of claim 120, wherein said two or more alleles are alleles from two or more different correlated clusters selected from those provided in Table 8.

122. A composition comprising quetiapine for use in treating a human subject having or at risk of developing SZ, the human subject having a haplotype tagged by an allele provided in Table 3A.

123. The composition of claim 122, wherein the human subject has a haplotype tagged by two or more alleles selected from those provided in Table 3A.

124. The composition of claim 123, wherein said two or more alleles are alleles from two or more different correlated clusters selected from those provided in Table 8.

125. An in vitro method of detecting the presence of a polymorphism in a human subject, the method comprising:
(a) detecting the haplotype tagged by an allele selected from those provided in Table 3B in a genomic sample from a human subject having or at risk of developing SZ; and
(b) identifying the subject having the haplotype tagged by the allele provided in Table 3B in the genomic sample as likely to have a poor response to quetiapine as compared to a control subject.

126. The method of claim 125 further comprising detecting the haplotype tagged by two or more alleles selected from those provided in Table 3B.

127. The method of claim 126, wherein said two or more alleles are alleles from two or more different correlated clusters selected from those provided in Table 8.

128. A composition comprising an antipsychotic agent that is not quetiapine for use in treating a human subject having or at risk of developing SZ, the human subject having a haplotype tagged by an allele provided in Table 3B.

129. The composition of claim 128, comprising olanzapine, perphenazine, risperidone or ziprasidone.

130. The composition of claim 128, wherein the human subject has a haplotype tagged by two or more alleles selected from those provided in Table 3B.

131. The composition of claim 130, wherein said two or more alleles are alleles from two or more different correlated clusters selected from those provided in Table 8.

132. An in vitro method of detecting the presence of a polymorphism in a human subject, the method comprising:
(a) detecting a haplotype tagged by the “C” allele of rs35793 in a genomic sample from a human subject having or at risk of developing SZ; and
(b) identifying the subject having the haplotype tagged by the “C” allele of rs35793in the genomic sample as likely to have poor response to quetiapine as compared to a control subject.

133. A composition comprising an antipsychotic agent that is not quetiapine for use in treating a human subject having or at risk of developing SZ, the human subject having a haplotype tagged by the “C” allele of rs35793.

134. The composition of claim 133, comprising olanzapine, perphenazine, risperidone or ziprasidone.

135. An in vitro method of detecting the presence of a polymorphism in a human subject, the method comprising:
(a) detecting the haplotype tagged by an allele selected from those provided in Table 4A in a genomic sample from a human subject having or at risk of developing SZ; and
(b) identifying the subject having the haplotype tagged by the allele provided in Table 4A in the genomic sample as likely to have an improved response to risperidone as compared to a control subject.

136. The method of claim 135, further comprising detecting the haplotype tagged by two or more alleles selected from those provided in Table 4A.

137. The method of claim 136, wherein said two or more alleles are alleles from two or more different correlated clusters selected from those provided in Table 9.

138. A composition comprising risperidone for use in treating a human subject having or at risk of developing SZ, the human subject having a haplotype tagged by an allele provided in Table 4A.

139. The composition of claim 138, wherein the human subject has a haplotype tagged by two or more alleles selected from those provided in Table 4A.

140. The composition of claim 139, wherein said two or more alleles are alleles from two or more different correlated clusters selected from those provided in Table 9.

141. An in vitro method of detecting the presence of a polymorphism in a human subject, the method comprising:
(a) detecting the haplotype tagged by an allele selected from those provided in Table 4B in a genomic sample from a human subject having or at risk of developing SZ; and
(b) identifying the subject having the haplotype tagged by the allele provided in Table 4B in the genomic sample as likely to have a poor response to risperidone as compared to a control subject.

142. The method of claim 141 further comprising detecting the haplotype tagged by two or more alleles selected from those provided in Table 4B.

143. The method of claim 142, wherein said two or more alleles are alleles from two or more different correlated clusters selected from those provided in Table 9.

144. A composition comprising an antipsychotic agent that is not risperidone for use in treating a human subject having or at risk of developing SZ, the human subject having a haplotype tagged by an allele provided in Table 4B.

145. The composition of claim 144, comprising olanzapine, perphenazine, quetiapine or ziprasidone.

146. The composition of claim 144, wherein the human subject has a haplotype tagged by two or more alleles selected from those provided in Table 4B.

147. The composition of claim 146, wherein said two or more alleles are alleles from two or more different correlated clusters selected from those provided in Table 9.

148. An in vitro method of detecting the presence of a polymorphism in a human subject, the method comprising:
(a) detecting a haplotype tagged by the “A” allele of rs9713, the haplotype tagged by the “C” allele of rs874295, or the haplotype tagged by the “C” allele of rs3738883 in a genomic sample from a human subject having or at risk of developing SZ; and
(b) identifying the subject having the haplotype tagged by the “A” allele of rs9713, the haplotype tagged by the “C” allele of rs874295, or the haplotype tagged by the “C” allele of rs3738883 in the genomic sample as likely to have an improved response to risperidone as compared to a control subject.

149. A composition comprising risperidone for use in treating a human subject having or at risk of developing SZ, the human subject having a haplotype tagged by the “A” allele of rs9713, the haplotype tagged by the “C” allele of rs874295, or the haplotype tagged by the “C” allele of rs3738883.

150. An in vitro method of detecting the presence of a polymorphism in a human subject, the method comprising:
(a) detecting a haplotype tagged by the “C” allele of rs 1869295 or the haplotype tagged by the “C” allele of rs1315115 in a genomic sample from a human subject having or at risk of developing SZ; and
(b) identifying the subject having the haplotype tagged by the “C” allele of rs1869295 or the haplotype tagged by the “C” allele of rs1315115 in the genomic sample as likely to have poor response to risperidone as compared to a control subject.

151. A composition comprising an antipsychotic agent that is not risperidone for use in treating a human subject having or at risk of developing SZ, the human subject having a haplotype tagged by the “C” allele of rs1869295 or the haplotype tagged by the “C” allele of rs1315115.

152. The composition of claim 151, comprising olanzapine, perphenazine, quetiapine or ziprasidone.

153. An in vitro method of detecting the presence of a polymorphism in a human subject, the method comprising:
(a) detecting the haplotype tagged by an allele selected from those provided in Table 5A in a genomic sample from a human subject having or at risk of developing SZ; and
(b) identifying the subject having the haplotype tagged by the allele provided in Table 5A in the genomic sample as likely to have an improved response to ziprasidone as compared to a control subject.

154. The method of claim 153, further comprising detecting the haplotype tagged by two or more alleles selected from those provided in Table 5A.

155. The method of claim 154, wherein said two or more alleles are alleles from two or more different correlated clusters selected from those provided in Table 10.

156. A composition comprising ziprasidone for use in treating a human subject having or at risk of developing SZ, the human subject having a haplotype tagged by an allele provided in Table 5A.

157. The composition of claim 156, wherein the human subject has a haplotype tagged by two or more alleles selected from those provided in Table 5A.

158. The composition of claim 157, wherein said two or more alleles are alleles from two or more different correlated clusters selected from those provided in Table 10.

159. An in vitro method of detecting the presence of a polymorphism in a human subject, the method comprising:
(a) detecting the haplotype tagged by an allele selected from those provided in Table 5B in a genomic sample from a human subject having or at risk of developing SZ; and
(b) identifying the subject having the haplotype tagged by the allele provided in Table 5B in the genomic sample as likely to have a poor response to ziprasidone as compared to a control subject.

160. The method of claim 159 further comprising detecting the haplotype tagged by two or more alleles selected from those provided in Table 5B.

161. The method of claim 160, wherein said two or more alleles are alleles from two or more different correlated clusters selected from those provided in Table 10.

162. A composition comprising an antipsychotic agent that is not ziprasidone for use in treating a human subject having or at risk of developing SZ, the human subject having a haplotype tagged by an allele provided in Table 5B.

163. The composition of claim 162, comprising olanzapine, perphenazine, quetiapine or risperidone.

164. The composition of claim 162, wherein the human subject has a haplotype tagged by two or more alleles selected from those provided in Table 5B.

165. The composition of claim 164, wherein said two or more alleles are alleles from two or more different correlated clusters selected from those provided in Table 10.

166. An in vitro method of detecting the presence of a polymorphism in a human subject, the method comprising:
(a) detecting a haplotype tagged by the “A” allele of rs4925300, the haplotype tagged by the “C” allele of rs1546519, or the haplotype tagged by the “A” allele of rs17194378 in a genomic sample from a human subject having or at risk of developing SZ; and
(b) identifying the subject having the haplotype tagged by the “A” allele of rs4925300, the haplotype tagged by the “C” allele of rs1546519, or the haplotype tagged by the “A” allele of rs17194378 in the genomic sample as likely to have an improved response to ziprasidone as compared to a control subject.

167. A composition comprising ziprasidone for use in treating a human subject having or at risk of developing SZ, the human subject having a haplotype tagged by the “A” allele of rs4925300, the haplotype tagged by the “C” allele of rs1546519, or the haplotype tagged by the “A” allele of rs17194378.

168. An in vitro method of detecting the presence of a polymorphism in a human subject, the method comprising:
(a) detecting a haplotype tagged by the “C” allele of rs9585618 in a genomic sample from a human subject having or at risk of developing SZ; and
(b) identifying the subject having the haplotype tagged by the “C” allele of rs9585618 in the genomic sample as likely to have poor response to ziprasidone as compared to a control subject.

169. A composition comprising an antipsychotic agent that is not ziprasidone for use in treating a human subject having or at risk of developing SZ, the human subject having a haplotype tagged by the “C” allele of rs9585618.

170. The composition of claim 169, comprising olanzapine, perphenazine, quetiapine or risperidone to the subject.

171. An in vitro assay method comprising:
(a) detecting two or more haplotypes tagged by alleles selected from those provided in Tables 1-5 in a genomic sample from a human subject having or at risk of developing SZ;
(c) calculating a predicted treatment efficacy for at least two drugs selected from the group consisting of olanzapine, perphenazine, quetiapine, risperidone, and ziprasidone; and
(d) ranking the predicted efficacy of the at least two drugs.

172. The method of claim 171, wherein detecting two or more haplotypes tagged by an allele comprises determining the number of alleles tagging the two or more haplotypes in the subject.

173. The method of claim 172, wherein calculating a predicted treatment efficacy for a given drug comprises assigning a weighted value to each haplotype influencing response to that drug and multiplying the weighted value by the number of alleles tagging the haplotype in the subject.

174. The method of claim 172, wherein calculating a predicted treatment efficacy for a given drug comprises using the equation:
P=C+ΣᵢβᵢNᵢ
wherein P is the predicted treatment efficacy measured in change in PANSS-T; C is the change in PANSS-T for individuals carrying zero alleles of any response-predicting haplotype for the drug, β is the weighted value for at least a first haplotype measured in PANSS-T; N is the number of alleles tagging at least the first haplotype; and i is the number of haplotypes detected.

175. The method of claim 171, comprising a predicted treatment efficacy for three, four or five drugs selected from the group consisting of olanzapine, perphenazine, quetiapine, risperidone, and ziprasidone.

176. The method or composition of any one of claims 83-175, wherein the subject has early, intermediate, or aggressive SZ.

177. The method or composition of any one of claims 83-175, wherein the subject has one or more risk factors associated with SZ.

178. The method or composition of any one of claims 83-175, wherein the haplotype tagged by an allele comprises determining the number of alleles tagging the haplotype in the subject.

179. The method or composition of any one of claims 83-175, wherein the subject has a relative afflicted with SZ or a genetically-based phenotypic trait associated with risk for SZ.

180. The method or composition of any one of claims 83-175, wherein the subject is Caucasian or comprises European ancestry.
